(19) United States
(12) Patent Application Publication
(10) Pub. No.: US 2010/0317704 A1
(43) Pub. Date: Dec. 16, 2010

(54) SPIRO-CONDENSED IMIDAZOLONE DERIVATIVES INHIBITING THE GLYCINE TRANSPORTER

(75) Inventors: Richard Blunt, Essex (GB); David Gwyn Cooper, Essex (GB); Roderick Alan Porter, Essex (GB); Paul Adrian Wyman, Essex (GB)

Correspondence Address: GlaxoSmithKline;GLOBAL PATENTS -US, UW2220, P. O. BOX 1539, KING OF PRUSSIA, PA, 19406-0939, US

(73) Assignee: Glaxo Group Limited a corporation

(21) Appl. No.: 12/676,625

(22) PCT Filed: Sep. 9, 2008
(86) PCT No.: PCT/EP2008/061889

§ 371 Date: Mar. 5, 2010

(30) Foreign Application Priority Data
Sep. 11, 2007 (GB) 0717728.0
Feb. 12, 2008 (GB) 0802586.8
Jul. 23, 2008 (GB) 0813501.4

(51) Int. Cl. A61K 31/4188; C07D 487/10; A61K 31/4184; A61P 25/28; A61P 25/18

(52) U.S. Cl. 514/387; 548/301.1

(57) ABSTRACT

Compounds of formula (I) and salts thereof are provided:

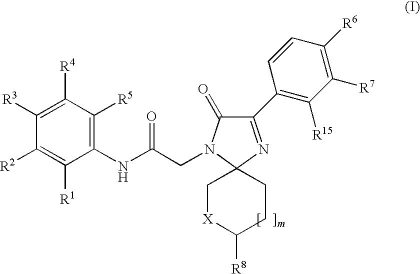

wherein the groups are as defined in the specification. Uses of the compounds as medicaments, and in the manufacture of medicament for treating neurological and neuropsychiatric disorders, in particular psychoses, dementia or attention deficit disorder are also disclosed. The invention further comprises processes to make these compounds and pharmaceutical formulations thereof.

The present invention relates to compounds, processes for their preparation, pharmaceutical compositions and medicaments containing them and to their use in the treatment of a condition wherein inhibition of GlyT1 would be beneficial, including neurological and neuropsychiatric disorders, in particular psychoses, dementia or attention deficit disorder.

Molecular cloning has revealed the existence in mammalian brains of two classes of glycine transporters, termed GlyT1 and GlyT2. GlyT1 is found predominantly in the forebrain and its distribution corresponds to that of glutaminergic pathways and NMDA receptors (Smith, et al., Neuron, 8, 1992: 927-935). Molecular cloning has further revealed the existence of three variants of GlyT1, termed GlyT-la, GlyT-1b and GlyT-1c (Kim et al., Molecular Pharmacology, 45, 1994: 608-617), each of which displays a unique distribution in the brain and peripheral tissues. The variants arise by differential splicing and exon usage, and differ in their N-terminal regions. GlyT2, in contrast, is found predominantly in the brain stem and spinal cord, and its distribution corresponds closely to that of strychnine-sensitive glycine receptors (Liu et al., J. Biological Chemistry, 268, 1993: 22802-22808; Jursky and Nelson, J. Neurochemistry, 64, 1995 : 1026-1033). Another distinguishing feature of glycine transport mediated by GlyT2 is that it is not inhibited by sarcosine as is the case for glycine transport mediated by GlyT1. These data are consistent with the view that, by regulating the synaptic levels of glycine, GlyT1 and GlyT2 selectively influence the activity of NMDA receptors and strychnine-sensitive glycine receptors, respectively.

NMDA receptors are critically involved in memory and learning (Rison and Staunton, Neurosci. Biobehav. Rev., 19 533-552 (1995); Danysz et al, Behavioral Pharmacol., 6 455-474 (1995)); and, furthermore, decreased function of NMDA-mediated neurotransmission appears to underlie, or contribute to, the symptoms of schizophrenia (Olney and Farber, Archives General Psychiatry, 52, 998-1007 (1996). Thus, agents that inhibit GlyT1 and thereby increase glycine activation of NMDA receptors can be used as novel antipsychotics and anti-dementia agents, and to treat other diseases in which cognitive processes are impaired, such as attention deficit disorders and organic brain syndromes. Conversely, over-activation of NMDA receptors has been implicated in a number of disease states, in particular the neuronal death associated with stroke and possibly neurodegenerative diseases, such as Alzheimer's disease, multi-infarct dementia, AIDS dementia, Huntington's disease, Parkinson's disease, amyotrophic lateral sclerosis or other conditions in which neuronal cell death occurs, such as stroke or head trauma. Coyle & Puttfarcken, Science, 262, 689-695 (1993); Lipton and Rosenberg, New Engl. J. of Medicine, 330, 613-622 (1993); Choi, Neuron, 1, 623-634 (1988). Thus, pharmacological agents that increase the activity of GlyT1 will result in decreased glycine-activation of NMDA receptors, which activity can be used to treat these and related disease states. Similarly, drugs that directly block the glycine site of the NMDA receptors can be used to treat these and related disease states.

Compounds which inhibit GlyT1 are known in the art, for example as disclosed in published international patent applications WO2007/104775 and WO2007/104776 (Glaxo Group Limited). However, there still remains the need to identify further compounds that can inhibit GlyT1 transporters, including those that inhibit GlyT1 transporters selectively over GlyT2 transporters.

In the first aspect, there is provided a compound of formula (I) or a salt thereof:

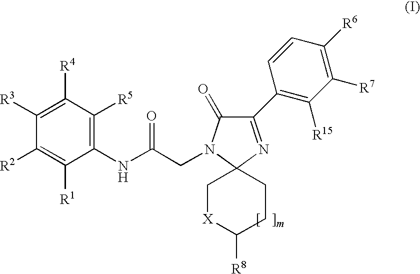

wherein:
X is —CH₂— or oxygen;
R¹, R², R³ and R⁴ are independently selected from hydrogen, C₁₋₄alkyl, C₁₋₄alkoxy, cyano, halo, haloC₁₋₄alkyl, haloC₁₋₄alkoxy, C₁₋₄alkylthio, C₃₋₆cycloalkyl, C₃₋₆cycloalkylC₁₋₄alkyl, C₃₋₆cycloalkylC₁₋₄alkoxy, C₁₋₄alkylsulfonyl, C₁₋₄alkoxyC₁₋₄alkyl and CONR^{a}R^{b} (wherein R^{a} and R^{b} are independently selected from hydrogen and C₁₋₄alkyl, or R^{a} and R^{b}, together with the nitrogen atom to which they are attached, form a 4- to 7-membered ring);
or R² and R³ together form a group selected from —O—CH₂—O— and —O—CH₂—CH₂—O—;
R⁵ is selected from hydrogen, chloro, fluoro, C₁₋₄alkyl and CF₃;
one of R⁶ and R⁷ is selected from the group consisting of:
   hydrogen, C₁₋₄alkyl, C₁₋₄alkoxy, haloC₁₋₄alkyl, haloC₁₋₄alkoxy, halo, cyano, C₁₋₄alkoxyC₁₋₄alkoxy and C₁₋₄alkoxyC₁₋₄alkyl;
and the other is selected from the group consisting of:
   a 5 to 7 membered heteroaryl ring, optionally substituted by C₁₋₄alkyl, C₁₋₄alkoxy, haloC₁₋₄alkyl, haloC₁₋₄alkoxy, halo or cyano;
   a 9 to 10 membered bicyclic heterocyclic ring, optionally substituted by C₁₋₄alkyl, C₁₋₄alkoxy, haloC₁₋₄alkyl, haloC₁₋₄alkoxy, halo or cyano; and
   a 5 to 7 membered heterocyclic ring, optionally substituted by C₁₋₄alkyl, C₁₋₄alkoxy, haloC₁₋₄alkyl, haloC₁₋₄alkoxy, halo or cyano;
or R⁶ and R⁷ together form a 5 to 7 membered heterocyclic ring fused to the phenyl ring, or a 5 to 7 membered heteroaryl ring fused to the phenyl ring; wherein the heterocyclic ring or the heteroaryl ring is optionally substituted by C₁₋₄alkyl, C₁₋₄alkoxy, haloC₁₋₄alkyl, haloC₁₋₄alkoxy, halo or cyano;
R¹⁵ is hydrogen or fluorine;
R⁸ is selected from hydrogen and methyl; and
m is selected from 0, 1 and 2.

The present invention also provides a compound of formula (Ia) or a salt thereof:

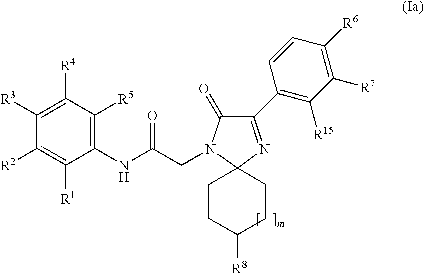

wherein:

R¹ is selected from H, C₁₋₄alkyl, C₁₋₄alkoxy, halo, haloC₁-C₄alkyl, haloC₁-C₄alkoxy, C₁-C₄alkylthio, C₃-C₆cycloalkyl, C₁-C₄alkylsulfonyl, C₁-C₄alkoxyC₁-C₄alkyl, CONR^{a}R^{b} (wherein R^{a} and R^{b} are independently selected from H and C₁-C₄alkyl, or R^{a} and R^{b}, together with the nitrogen atom to which they are attached, form a 4- to 7-membered ring) and cyano;

R² is selected from H, C₁-C₄alkyl, C₁-C₄alkoxy, halo, haloC₁-C₄alkyl, haloC₁-C₄alkoxy, C₁-C₄alkylthio, C₃-C₆cycloalkyl, C₁-C₄alkylsulfonyl, C₁-C₄alkoxyC₁-C₄alkyl, CONR^{c}R^{d}, (wherein R^{c} and R^{d} are independently selected from H and C₁-C₄alkyl, or R^{c} and R^{d}, together with the nitrogen atom to which they are attached, form a 4- to 7-membered ring) and cyano;

R³ is selected from H, methyl C1-4alkyl, C₁-C₄alkoxy, halo, haloC₁-C₄alkyl, haloC₁-C₄alkoxy, C₁-C₄alkylthio, C₃-C₆cycloalkyl, C₁-C₄alkylsulfonyl, C₁-C₄alkoxyC₁-C₄alkyl, CONR^{e}R^{f} (wherein R^{e} and R^{f} are independently selected from H and C₁-C₄alkyl, or R^{e} and R^{f}, together with the nitrogen atom to which they are attached, form a 4- to 7-membered ring) and cyano;

or R² and R³ together form a group selected from —O—CH₂—O— and —O—CH₂—CH₂—O—;

R⁴ is selected from H, C₁-C₄alkyl, C₁-C₄alkoxy, halo, haloC₁-C₄alkyl, haloC₁-C₄alkoxy, C₁-C₄alkylthio, C₃-C₆cycloalkyl, C₁-C₄alkylsulfonyl, C₁-C₄alkoxyC₁-C₄alkyl, CONR^{g}R^{h} (wherein R^{g} and R^{h} are independently selected from H and C₁-C₄alkyl, or R^{g} and R^{h}, together with the nitrogen atom to which they are attached, form a 4- to 7-membered ring) and cyano;

R⁵ is selected from hydrogen, chloro, fluoro, C₁-C₄alkyl and CF₃;

R⁶ is selected from a 5-7 membered heteroaryl or 5 to 7 membered heterocyclic ring optionally substituted by C₁-₄ alkyl, C₁-₄ alkoxy, haloalkyl, haloalkoxy, halo or cyano;

R¹⁵ is H or F;

R⁷ is selected from H, C₁C₄alkyl, C₁-C₄alkoxy, haloC₁-C₄alkyl, haloC₁-C₄alkoxy, halo, cyano, C₁-C₄alkoxyC₁-C₄alkoxy and C₁₋₄alkoxyC₁₋₄alkyl;

R⁸ is selected from hydrogen and methyl; and

m is selected from 0, 1 and 2.

The present invention also provides provided a compound of formula (Ib) or a salt thereof:

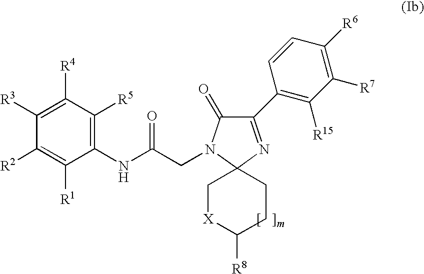

wherein:
X is —CH₂— or oxygen;
R¹, R², R³ and R⁴ are independently selected from hydrogen, C₁₋₄alkyl, C₁₋₄alkoxy, cyano, halo, haloC₁-C₄alkyl, haloC₁-C₄alkoxy, C₁-C₄alkylthio, C₃-C₆cycloalkyl, C₁-C₄alkylsulfonyl, C₁-C₄alkoxyC₁-C₄alkyl, CONR^{a}R^{b} (wherein R^{a} and R^{b} are independently selected from H and C₁-C₄alkyl, or R^{a} and R^{b}, together with the nitrogen atom to which they are attached, form a 4- to 7-membered ring);
or R² and R³ together form a group selected from —O—CH₂—O— and —O—CH₂—CH₂—O—;
R⁵ is selected from hydrogen, chloro, fluoro, C₁-C₄alkyl and CF₃;
one of R⁶ and R⁷ is selected from the group consisting of:
   hydrogen, C₁₋₄alkyl, C₁₋₄alkoxy, haloC₁₋₄alkyl, haloC₁₋₄alkoxy, halo, cyano, C₁₋₄alkoxyC₁₋₄alkoxy and C₁₋₄alkoxyC₁₋₄alkyl;
and the other is selected from the group consisting of:
   a 5 to 7 membered heteroaryl ring, optionally substituted by C₁₋₄alkyl, C₁₋₄alkoxy, haloC₁₋₄alkyl, haloC₁₋₄alkoxy, halo or cyano;
   a 9 to 10 membered bicyclic heterocyclic ring, optionally substituted by C₁₋₄alkyl, C₁₋₄alkoxy, haloC₁₋₄alkoxy, haloC₁₋₄alkoxy, halo or cyano; and
   a 5 to 7 membered heterocyclic ring, optionally substituted by C₁₋₄alkyl, C₁₋₄alkoxy, haloC₁₋₄alkyl, haloC₁₋₄alkoxy, halo or cyano;
or R⁶ and R⁷ together form a 5 to 7 membered heterocyclic ring fused to the phenyl ring, or a 5 to 7 membered heteroaryl ring fused to the phenyl ring; wherein the heterocyclic ring or the heteroaryl ring is optionally substituted by C₁₋₄alkyl, C₁₋₄alkoxy, haloC₁₋₄alkyl, haloC₁₋₄alkoxy, halo or cyano;
RP^{15P} is hydrogen or fluorine;
RP^{8P} is selected from hydrogen and methyl; and
m is selected from 0, 1 and 2.

The notations “Cₓ-C_{y}” and “C_{x-y}” are interchangeable.

As used herein, the term “C₁₋₄alkyl” refers to a straight or branched alkyl group of 1, 2, 3 or 4 carbon atoms in all isomeric forms. Examples include methyl, ethyl, propyl, isopropyl, butyl, isobutyl, sec-butyl and tert-butyl.

As used herein, the term “C₁₋₄alkoxy” refers to the group —O—C₁₋₄alkyl wherein C₁₋₄alkyl is as defined above.

As used herein, the term “C₁₋₄alkoxyC₁₋₄alkyl” refers to the group —C₁₋₄alkyl-O—C₁₋₄alkyl, wherein C₁₋₄alkyl is as defined above.

As used herein, the term “C₁₋₄alkoxyC₁₋₄alkoxy” refers to the group —O—C₁₋₄alkyl-O—C₁₋₄alkyl, wherein C₁₋₄alkyl is as defined above.

As used herein, the term “C₃₋₆cycloalkyl” refers to a cycloalkyl group consisting of from 3 to 6 carbon atoms, ie cyclopropanyl, cyclobutanyl, cyclopentanyl and cyclohexanyl.

As used herein, the terms “halogen” and its abbreviation “halo” refer to fluorine, chlorine, bromine, or iodine.

As used herein, the term “haloC₁₋₄alkyl” refers to a C₁₋₄alkyl group as defined above which is substituted with any number of fluorine, chlorine, bromine, or iodine atoms, including with mixtures of those atoms. A haloC₁₋₄alkyl group may, for example contain 1, 2 or 3 halogen atoms. For example, a haloC₁₋₄alkyl group may have all hydrogen atoms replaced with halogen atoms. Examples of haloC₁₋₄alkyl groups include, but are not limited to, fluoromethyl, difluoromethyl and trifluoromethyl.

As used herein, the term “haloC₁₋₄alkoxy” refers to a C₁₋₄alkoxy group as defined above which is substituted with any number of fluorine, chlorine, bromine, or iodine atoms, including with mixtures of those atoms. A haloC₁₋₄alkoxy group may, for example contain 1, 2 or 3 halogen atoms. For example, a haloC₁₋₄alkoxy group may have all hydrogen atoms replaced with halogen atoms. Examples of haloC₁₋₄alkoxy groups include, but are not limited to, fluoromethyloxy, difluoromethyloxy and trifluoromethyloxy.

As used herein the term “cyano” refers to a group —CN.

As used herein, the term “C₁₋₄alkylsulfonyl” refers to a group —SO₂—C₁₋₄alkyl. An example is —SO₂CH₃.

As used herein, the term “C₁₋₄alkylthio” refers to a group —S—C₁₋₄alkyl. An example is —SCH₃.

As used herein, the term “5 to 7 membered heteroaryl” refers to an aromatic ring consisting of 5, 6 or 7 carbon atoms, wherein 1 to 4 carbon atoms are replaced by a heteroatom selected from oxygen, nitrogen and sulphur. Examples of such aromatic rings include thienyl, furyl, pyrrolyl, triazolyl, imidazolyl, pyrazolyl, pyridyl, pyrimidinyl, pyridazinyl, pyridazyl, triazinyl and tetrazolyl. The heteroaryl ring may be linked to the remainder of the molecule via a carbon atom, or when present, a suitable nitrogen atom.

As used herein, the term “9 to 10 membered bicyclic heterocyclic ring” refers to a bicyclic aromatic or non-aromatic system consisting of 9 or 10 carbon atoms, wherein 1 to 4 carbon atoms are replaced by a heteroatom selected from oxygen, nitrogen and sulphur. Examples of bicyclic heterocyclic rings include quinolinyl, isoquinolinyl, indolyl, benzofuryl, benzothienyl, benzoimidazolyl, benzoxazolyl, tetrahydroquinolinyl, tetrahydroisoquinolinyl, indolinyl, dihydrobenzofuranyl and dihydrobenzothiophenyl. The bicyclic system may be linked to the remainder of the molecule via a carbon atom, or when present, a suitable nitrogen atom.

As used herein, the term “5 to 7 membered heterocyclic ring” refers to a non-aromatic ring consisting of 5, 6 or 7 carbon atoms, wherein 1 to 4 carbon atoms are replaced by a heteroatom selected from oxygen, nitrogen and sulphur. Examples of such non-aromatic rings include piperidinyl, piperazinyl, pyrrolidinyl and morpholinyl. The heterocyclic ring may be linked to the remainder of the molecule via a carbon atom, or when present, a suitable nitrogen atom.

When R⁶ and R⁷ together form a 5 to 7 membered heterocyclic ring fused to the phenyl ring, examples of the resulting fused system include tetrahydroquinolinyl, tetrahydroisoquinolinyl, indolinyl, dihydrobenzofuranyl and dihydrobenzothiophenyl. When R⁶ and R⁷ together form a 5 to 7 membered heteroaryl ring fused to the phenyl ring, examples of the resulting fused ring system include quinolinyl, isoquinolinyl, indolyl, benzofuryl, benzothienyl, benzoimidazolyl and benzoxazolyl.

In one embodiment R¹ is selected from H, C₁₋₂alkyl, C₁₋₂alkoxy, halo, haloC₁₋₂alkyl, haloC₁₋₂alkoxy, C₁₋₂alkylthio, C₁₋₂alkylsulfonyl, C₁₋₂alkoxyC₁₋₂alkyl, and cyano. In a further embodiment R¹ is H.

In one embodiment R² is selected from H, C₁₋₂alkyl, C₁₋₂alkoxy, halo, haloC₁₋₂alkyl, haloC₁₋₂alkoxy, C₁₋₂alkylthio, C₁₋₂alkylsulfonyl, C₁₋₂alkoxyC₁₋₂alkyl, and cyano. In a further embodiment R² is halo or haloC₁₋₂alkyl. In a further embodiment R² is F or CF₃.

In one embodiment R³ is selected from H, C₁₋₂alkyl, C₁₋₂alkoxy, halo, haloC₁₋₂alkyl, haloC₁₋₂alkoxy, C₁₋₂alkylthio, C₁₋₂alkylsulfonyl, C₁₋₂alkoxyC₁₋₂alkyl, and cyano. In a further embodiment R³ is H.

In one embodiment R⁴ is selected from H, C₁₋₂alkyl, C₁₋₂alkoxy, halo, haloC₁₋₂alkyl, haloC₁₋₂alkoxy, C₁₋₂alkylthio, C₁₋₂alkylsulfonyl, C₁₋₂alkoxyC₁₋₂alkyl, and cyano. In a further embodiment R⁴ is hydrogen or halo. In a further embodiment R⁴ is F, H or Cl.

In one embodiment R⁵ is H.

In one embodiment:

R¹, R³ and R⁵ are all H;

R² is F or CF₃; and

R⁴ is F or H.

In one embodiment R⁶ is selected from H, C₁₋₂alkyl, C₁₋₂alkoxy, haloC₁₋₂alkyl, haloC₁₋₂alkoxy, halo, cyano, C₁₋₂alkoxyC₁₋₂alkoxy and C₁₋₂alkoxyC₁₋₂alkyl. In a further embodiment R⁶ is H.

In one embodiment, R⁶ is selected from the group consisting of:
a 5 to 7 membered heteroaryl ring, optionally substituted by C₁₋₄alkyl, C₁₋₄alkoxy, haloC₁₋₄alkyl, haloC₁₋₄alkoxy, halo or cyano;
a 9 to 10 membered bicyclic heterocyclic ring, optionally substituted by C₁₋₄alkyl, C₁₋₄alkoxy, haloC₁₋₄alkyl, haloC₁₋₄alkoxy, halo or cyano; and
a 5 to 7 membered heterocyclic ring, optionally substituted by C₁₋₄alkyl, C₁₋₄alkoxy, haloC₁₋₄alkyl, haloC₁₋₄alkoxy, halo or cyano.

In one embodiment, R⁶ is a 5 to 7 membered heteroaryl ring, optionally substituted by C₁₋₄alkyl, C₁₋₄alkoxy, haloC₁₋₄alkyl, haloC₁₋₄alkoxy, halo or cyano.

In one embodiment, R⁶ is a 5 or 6 membered heteroaryl ring, optionally substituted by C₁₋₄alkyl, C₁₋₄alkoxy, haloC₁₋₄alkyl, haloC₁₋₄alkoxy, halo or cyano.

In one embodiment R⁶ is imidazolyl, optionally substituted by C₁₋₄alkyl (such as methyl or propyl).

In one embodiment, R⁶ is pyridyl.

In one embodiment R⁷ is selected from H, C₁₋₂alkyl, C₁₋₂alkoxy, haloC₁₋₂alkyl, haloC₁₋₂alkoxy, halo, cyano, C₁₋₂alkoxyC₁₋₂alkoxy and C₁₋₂alkoxyC₁₋₂alkyl. In one embodiment R⁷ is H.

In one embodiment, R⁷ is selected from the group consisting of:
a 5 to 7 membered heteroaryl ring, optionally substituted by C₁₋₄alkyl, C₁₋₄alkoxy, haloC₁₋₄alkyl, haloC₁₋₄alkoxy, halo or cyano;
a 9 to 10 membered bicyclic heterocyclic ring, optionally substituted by C₁₋₄alkyl, C₁₋₄alkoxy, haloC₁₋₄alkyl, haloC₁₋₄alkoxy, halo or cyano; and
a 5 to 7 membered heterocyclic ring, optionally substituted by C₁₋₄alkyl, C₁₋₄alkoxy, haloC₁₋₄alkyl, haloC₁₋₄alkoxy, halo or cyano.

In one embodiment, R⁷ is a 5 to 7 membered heteroaryl ring, optionally substituted by C₁₋₄alkyl, C₁₋₄alkoxy, haloC₁₋₄alkyl, haloC₁₋₄alkoxy, halo or cyano.

In one embodiment, R⁷ is a 5 or 6 membered heteroaryl ring, optionally substituted by C₁₋₄alkyl, C₁₋₄alkoxy, haloC₁₋₄alkyl, haloC₁₋₄alkoxy, halo or cyano.

In one embodiment R⁷ is imidazolyl, optionally substituted by C₁₋₄alkyl (such as methyl or propyl).

In one embodiment, R⁷ is pyridyl.

In one embodiment, R⁷ is H and R⁶ is a 5 or 6 membered heteroaryl ring, optionally substituted by C₁₋₄alkyl, C₁₋₄alkoxy, haloC₁₋₄alkyl, haloC₁₋₄alkoxy, halo or cyano. In one embodiment, R⁶ is H and R⁷ is a 5 or 6 membered heteroaryl ring, optionally substituted by C₁₋₄alkyl, C₁₋₄alkoxy, haloC₁₋₄alkyl, haloC₁₋₄alkoxy, halo or cyano.

In one embodiment R¹⁵ is H.

In one embodiment R⁸ is H.

In one embodiment m is 1. In one embodiment, m is 0.

The present invention also provides a compound of formula (Ic) or a salt thereof:

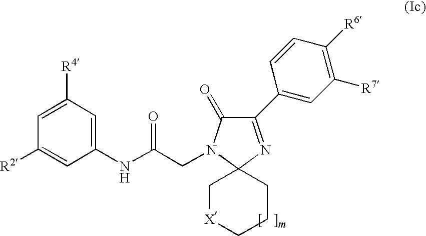

wherein:
X′ is —CH₂— or oxygen;
R^{2′} and R^{4′} are independently selected from hydrogen, C₁₋₄alkyl, C₁₋₄alkoxy, cyano, halo, haloC₁₋₄alkyl, haloC₁₋₄alkoxy, C₁₋₄alkylthio, C₃₋₆cycloalkyl, C₃₋₆cycloalkylC₁₋₄alkyl, C₃₋₆cycloalkylC₁₋₄alkoxy, C₁₋₄alkylsulfonyl, C₁₋₄alkoxyC₁₋₄alkyl, CONR^{a}R^{b} (wherein R^{a} and R^{b} are independently selected from hydrogen and C₁₋₄alkyl, or R^{a} and R^{b}, together with the nitrogen atom to which they are attached, form a 4- to 7-membered ring);
one of R^{6′} and R^{7′} is selected from the group consisting of:
   hydrogen, C₁₋₄alkyl, C₁₋₄alkoxy, haloC₁₋₄alkyl, haloC₁₋₄alkoxy, halo, cyano, C₁₋₄alkoxyC₁₋₄alkoxy, C₁₋₄alkoxyC₁₋₄alkyl;
and the other is selected from the group consisting of:
   a 5 to 7 membered heteroaryl ring, optionally substituted by C₁₋₄alkyl, C₁₋₄alkoxy, haloC₁₋₄alkyl, haloC₁₋₄alkoxy, halo or cyano;
   a 9 to 10 membered bicyclic heterocyclic ring, optionally substituted by C₁₋₄alkyl, C₁₋₄alkoxy, haloC₁₋₄alkyl, haloC₁₋₄alkoxy, halo or cyano; and
   a 5 to 7 membered heterocyclic ring, optionally substituted by C₁₋₄alkyl, C₁₋₄alkoxy, haloC₁₋₄alkyl, haloC₁₋₄alkoxy, halo or cyano;
or R^{6′} and R^{7′} together form a 5 to 7 membered heterocyclic ring fused to the phenyl ring, or a 5 to 7 membered heteroaryl ring fused to the phenyl ring; wherein the heterocyclic ring or the heteroaryl ring is optionally substituted by C₁₋₄alkyl, C₁₋₄alkoxy, haloC₁₋₄alkyl, haloC₁₋₄alkoxy, halo or cyano; and
m is selected from 0, 1 and 2.

In one embodiment, R^{2′} and R^{4′} are independently selected from hydrogen, halo and haloC₁₋₄alkyl.

In one embodiment, one of R^{6′} and R^{7′} is hydrogen and the other is a 5 to 7 membered heteroaryl ring, optionally substituted by C₁₋₄alkyl, C₁₋₄alkoxy, haloC₁₋₄alkyl, haloC₁₋₄alkoxy, halo or cyano.

In one embodiment, m′ is selected from 0 and 1.

It will be appreciated that the present invention is intended to include compounds having any combination of the groups listed hereinbefore. It will be understood that, where appropriate, an embodiment described above for one part of the invention may be combined with an embodiment of another part of the invention.

For the avoidance of doubt, unless otherwise indicated, the term “substituted” means substituted by one or more defined groups. In the case where groups may be selected from a number of alternative groups, the selected groups may be the same or different. For the avoidance of doubt, the term “independently” means that where more than one substituent is selected from a number of possible substituents, those substituents may be the same or different.

Examples of compounds of the invention include:

2-{3-[4-(4-Methyl-1H-imidazol-1-yl)phenyl]-2-oxo-1,4-diazaspiro[4.4]non-3-en-1-yl}-N-[3-(trifluoromethyl)phenyl]acetamide

2-{3-[4-(5-Methyl-1H-imidazol-1-yl)phenyl]-2-oxo-1,4-diazaspiro[4.4]non-3-en-1-yl}-N-[3-(trifluoromethyl)phenyl]acetamide

2-{3-[4-(4-methyl-1H-imidazol-1-yl)phenyl]-2-oxo-1,4-diazaspiro[4.5]dec-3-en-1-yl}-N-[3-(trifluoromethyl)phenyl]acetamide

2-{3-[4-(5-methyl-1H-imidazol-1-yl)phenyl]-2-oxo-1,4-diazaspiro[4.5]dec-3-en-1-yl}-N-[3-(trifluoromethyl)phenyl]acetamide

2-{3-[4-(1H-imidazol-1-yl)phenyl]-2-oxo-1,4-diazaspiro[4.5]dec-3-en-1-yl}-N-[3-(trifluoromethyl)phenyl]acetamide

2-{3-[4-(2-methyl-1H-imidazol-1-yl)phenyl]-2-oxo-1,4-diazaspiro[4.5]dec-3-en-1-yl}-N-[3-(trifluoromethyl)phenyl]acetamide

2-{2-Oxo-3-[4-(2-propyl-1H-imidazol-1-yl)phenyl]-1,4-diazaspiro[4.5]dec-3-en-1-yl}-N-[3-(trifluoromethyl)phenyl]acetamide

N-(3,5-Difluorophenyl)-2-{3-[4-(4-methyl-1H-imidazol-1-yl)phenyl]-2-oxo-1,4-diazaspiro[4.4]non-3-en-1-yl}acetamide

2-{3-[4-(4-Methyl-1H-imidazol-1-yl)phenyl]-2-oxo-7-oxa-1,4-diazaspiro[4.4]non-3-en-1-yl}-N-[3-(trifluoromethyl)phenyl]acetamide

2-{3-[4-(4-Methyl-1H-imidazol-1-yl)phenyl]-2-oxo-7-oxa-1,4-diazaspiro[4.5]dec-3-en-1-yl}-N-[3-(trifluoromethyl)phenyl]acetamide

2-{3-[4-(5-methyl-1H-imidazol-1-yl)phenyl]-2-oxo-7-oxa-1,4-diazaspiro[4.5]dec-3-en-1-yl}-N-[3-(trifluoromethyl)phenyl]acetamide

2-{2-Oxo-3-[4-(2-pyridinyl)phenyl]-7-oxa-1,4-diazaspiro[4.4]non-3-en-1-yl}-N-[3-(trifluoromethyl)phenyl]acetamide

2-{2-Oxo-3-[4-(2-pyridinyl)phenyl]-1,4-diazaspiro[4.4]non-3-en-1-yl}-N-[3-(trifluoromethyl)phenyl]acetamide

N-(3-Chlorophenyl)-2-{3-[4-(4-methyl-1H-imidazol-1-yl)phenyl]-2-oxo-1,4-diazaspiro[4.4]non-3-en-1-yl}acetamide

2-{2-Oxo-3-[3-(2-pyridinyl)phenyl]-1,4-diazaspiro[4.5]dec-3-en-1-yl}-N-[3-(trifluoromethyl)phenyl]acetamide

2-{2-Oxo-3-[4-(2-pyridinyl)phenyl]-7-oxa-1,4-diazaspiro[4.5]dec-3-en-1-yl}-N-[3-(trifluoromethyl)phenyl]acetamide

N-(3,5-Difluorophenyl)-2-{2-oxo-3-[4-(2-pyridinyl)phenyl]-7-oxa-1,4-diazaspiro[4.5]dec-3-en-1-yl}acetamide

2-{3-[4-(2-Methyl-1H-imidazol-1-yl)phenyl]-2-oxo-1,4-diazaspiro[4.4]non-3-en-1-yl}-N-[3-(trifluoromethyl)phenyl]acetamide

2-{3-[4-(2,4-Dimethyl-1H-imidazol-1-yl)phenyl]-2-oxo-1,4-diazaspiro[4.4]non-3-en-1-yl}-N-[3-(trifluoromethyl)phenyl]acetamide

2-{3-[4-(4,5-Dimethyl-1H-imidazol-1-yl)phenyl]-2-oxo-1,4-diazaspiro[4.4]non-3-en-1-yl}-N-[3-(trifluoromethyl)phenyl]acetamide

2-{3-[3-(4-Methyl-1H-imidazol-1-yl)phenyl]-2-oxo-1,4-diazaspiro[4.5]dec-3-en-1-yl}-N-[3-(trifluoromethyl)phenyl]acetamide

2-{3-[3-(5-Methyl-1H-imidazol-1-yl)phenyl]-2-oxo-1,4-diazaspiro[4.5]dec-3-en-1-yl}-N-[3-(trifluoromethyl)phenyl]acetamide

2-{3-[4-(1-Methyl-1H-imidazol-2-yl)phenyl]-2-oxo-1,4-diazaspiro[4.4]non-3-en-1-yl}-N-[3-(trifluoromethyl)phenyl]acetamide and salts thereof.

Salts of compounds of formula (I) which are suitable for use in medicine are those wherein the counterion is pharmaceutically acceptable. However, salts having non-pharmaceutically acceptable counterions are within the scope of the present invention, for example, for use as intermediates in the preparation of other compounds of formula (I) and their pharmaceutically acceptable salts and/or for use in non-therapeutic, for example, in vitro, situations.

As used herein, the term “salt” refers to any salt of a compound according to the present invention prepared from an inorganic or organic acid or base, quaternary ammonium salts and internally formed salts. Pharmaceutically acceptable salts are particularly suitable for medical applications because of their greater aqueous solubility relative to the parent compounds. Such salts must clearly have a pharmaceutically acceptable anion or cation. Suitably pharmaceutically acceptable salts of the compounds of the present invention include acid addition salts formed with inorganic acids such as hydrochloric, hydrobromic, hydroiodic, phosphoric, metaphosphoric, nitric and sulfuric acids, and with organic acids, such as tartaric, acetic, trifluoroacetic, citric, malic, lactic, fumaric, benzoic, formic, propionic, glycolic, gluconic, maleic, succinic, (1R)-(−)-10-camphorsulphonic, (1S)-(+)-10-camphorsulphonic, isothionic, mucic, gentisic, isonicotinic, saccharic, glucuronic, furoic, glutamic, ascorbic, anthranilic, salicylic, phenylacetic, mandelic, embonic (pamoic), methanesulfonic, ethanesulfonic, pantothenic, stearic, sulfinilic, alginic, galacturonic and arylsulfonic, for example naphthalene-1,5-disulphonic, naphthalene-1,3-disulphonic, benzenesulfonic, and p-toluenesulfonic, acids. The salts may have any suitable stoichiometry. For example, a salt may have 1:1 or 2:1 stoichiometry. Non-integral stoichiometry ratios are also possible.

In an embodiment there is provided a compound of formula (I) as defined above or a pharmaceutically acceptable salt thereof.

In one embodiment, there is provided a compound of formula (I) as defined above or a hydrochloride or a formate salt thereof.

Solvates of the compounds of formula (I) and solvates of the salts of the compounds of formula (I) are included within the scope of the present invention. As used herein, the term “solvate” refers to a complex of variable stoichiometry formed by a solute (in this invention, a compound of formula (I) or a salt thereof) and a solvent. Those skilled in the art of organic chemistry will appreciate that many organic compounds can form such complexes with solvents in which they are reacted or from which they are precipitated or crystallized. Such solvents for the purpose of the invention may not interfere with the biological activity of the solute. Examples of suitable solvents include, but are not limited to, water, methanol, ethanol and acetic acid. Preferably the solvent used is a pharmaceutically acceptable solvent. Examples of suitable pharmaceutically acceptable solvents include, without limitation, water, ethanol and acetic acid. Most preferably the solvent used is water. Where the solvent used is water such a solvate may then also be referred to as a hydrate.

It will be appreciated by those skilled in the art that certain protected derivatives of compounds of formula (I), which may be made prior to a final deprotection stage, may not possess pharmacological activity as such, but may, in certain instances, be administered orally or parenterally and thereafter metabolised in the body to form compounds of the invention which are pharmacologically active. Such derivatives may therefore be described as “prodrugs”. Further, certain compounds of the invention may be administered as prodrugs. Examples of pro-drug forms for certain compounds of the present invention are described in Drugs of Today, Volume 19, Number 9, 1983, pp 499-538 and in Topics in Chemistry, Chapter 31, pp 306-316 and in “Design of Prodrugs” by H. Bundgaard, Elsevier, 1985, Chapter 1 (the disclosures in which documents are incorporated herein by reference). It will further be appreciated by those skilled in the art, that certain moieties, known to those skilled in the art as “pro-moieties”, for example as described by H. Bundgaard in “Design of Prodrugs” (the disclosure in which document is incorporated herein by reference) may be placed on appropriate functionalities when such functionalities are present within compounds of the invention. Examples of prodrugs for certain compounds of the invention include: esters, carbonate esters, hemi-esters, phosphate esters, sulfate esters, sulfoxides, amides, carbamates, phosphamides, glycosides and ethers.

Hereinafter, compounds of formula (I) (whether in solvated or unsolvated form) or their pharmaceutically acceptable salts (whether in solvated or unsolvated form) or prodrugs thereof defined in any aspect of the invention (except intermediate compounds in chemical processes) are referred to as “compounds of the invention”.

Also included within the scope of the invention are polymorphs of a compound of the invention.

The invention also includes all suitable isotopic variations of a compound of the invention. An isotopic variation of a compound of the invention is defined as one in which at least one atom is replaced by an atom having the same atomic number but an atomic mass different from the atomic mass usually found in nature. Examples of isotopes that can be incorporated into compounds of the invention include isotopes of hydrogen, carbon, nitrogen, oxygen, phosphorus, sulphur, fluorine and chlorine such as ²H, ³H, ¹¹C, ¹³C, ¹⁴C, ¹⁵N, ¹⁷O, ¹⁸O, ³¹P, ³²P, ³⁵S ¹⁸F, and ³⁶Cl, respectively. Certain isotopic variations of the invention, for example, those in which a radioactive isotope such as ³H or ¹⁴C is incorporated, are useful in drug and/or substrate tissue distribution studies. Tritiated, i.e., ³H, and carbon-14, i.e., ¹⁴C, u isotopes are particularly preferred for their ease of preparation and detectability. Further, substitution with isotopes such as deuterium, i.e., ²H, may afford certain therapeutic advantages resulting from greater metabolic stability, for example, increased in vivo half-life or reduced dosage requirements and hence may be preferred in some circumstances. Isotopic variations of the compounds of the invention can generally be prepared by conventional procedures such as by the illustrative methods or by the preparations described in the Examples hereafter using appropriate isotopic variations of suitable reagents.

Certain of the compounds described herein may exist in stereoisomeric forms (i.e. they may contain one or more asymmetric carbon atoms or may exhibit cis-trans isomerism). For example, there is at least one chiral centre when X in formula (I) is oxygen as shown below:

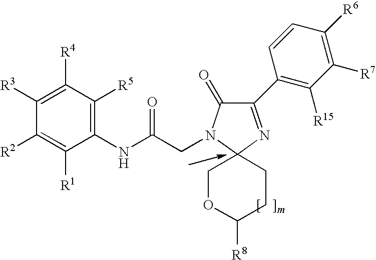

The individual stereoisomers (enantiomers and diastereoisomers) and mixtures of these are included within the scope of the present invention. When a compound is desired as a single enantiomer, it may be obtained by stereospecific synthesis or by resolution of the final product or any convenient intermediate. Resolution of the final product, an intermediate, or a starting material may be effected by any suitable method known in the art such as high-performance liquid chromatography or other appropriate means. See, for example, Stereochemistry of Organic Compounds by E. L. Eliel, S. H. Wilen, and L. N. Mander (Wiley-Interscience, 1994). It is also understood that compounds of formula (I) may exist in tautomeric forms other than that shown in the formula and these are also included within the scope of the present invention.

In one embodiment, an optically pure enantiomer of a compound of the present invention is provided. The term “optically pure enantiomer” means that the compound contains greater than about 90% of the desired isomer by weight, such as greater than about 95% of the desired isomer by weight, or greater than about 99% of the desired isomer by weight, said weight percent based upon the total weight of the isomer(s) of the compound.

Compounds of general formula (I) and their salts may be prepared by methods known in the art of organic synthesis as set forth in part by the following synthesis schemes. It is also recognised that in all of the schemes described below, it is well understood that protecting groups for sensitive or reactive groups are employed where necessary in accordance with general principles of chemistry. Protecting groups are manipulated according to standard methods of organic synthesis (T. W. Greene and P. G. M. Wuts (1991) Protecting Groups in Organic Synthesis, John Wiley & Sons). These groups are removed at a convenient stage of the compound synthesis using methods that are readily apparent to those skilled in the art. The selection of processes as well as the reaction conditions and order of their execution shall be consistent with the preparation of compounds of formula (I).

Typical reaction routes for the preparation of a compound of formula (I) as hereinbefore defined, are shown below. In the schemes below, X in formula (I) is shown to be —CH₂— but may also be oxygen.

Compounds of formula (I) can be prepared by reacting a compound of formula (II) with a base, for example sodium hydride, in a suitable inert solvent, for example dimethylformamide, followed by treatment with a compound of formula (III) where X is halogen as shown in Scheme 1.

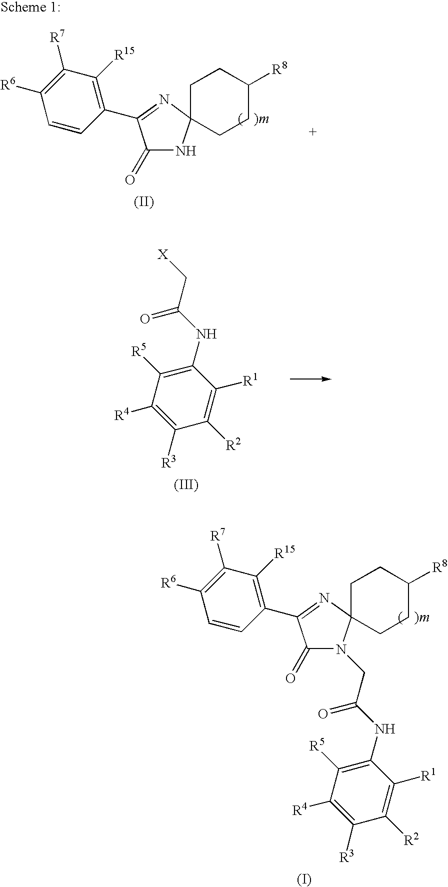

Compounds of formula (III) can be prepared by standard methods, for example as shown in Scheme 2. For example, an aniline of formula (IV) may be combined with an haloacetyl halide of formula (XII) where X and X′ are halogen, for example chloroacetyl chloride or bromoacetyl bromide in an inert solvent, for example dichloromethane at reduced then ambient temperature with subsequent addition of a base such as sodium bicarbonate. Alternatively the reaction may be carried out in an inert solution, for example, dioxan and heated to give a compound of formula (III).

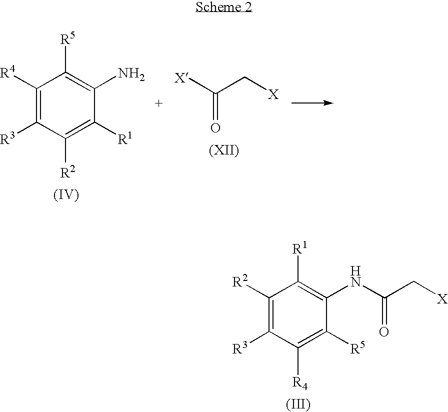

Compounds of formula (II) may be prepared by desulphurisation of compounds of formula (V) using an oxidising agent, for example hydrogen peroxide as shown for example in Scheme 3.

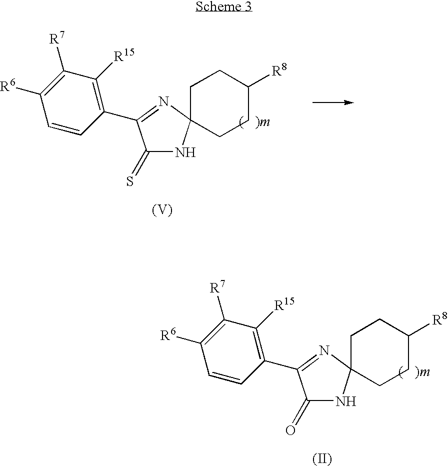

Compounds of formula (V) can be prepared by treating a ketothioamide of formula (VI) with the appropriate ketone of formula (VII) in the presence of a source of ammonia, for example ammonium acetate as shown in Scheme 4. Preferably this reaction is performed in a solvent, for example isopropanol, at room or elevated temperature, preferably elevated temperature, for example at reflux.

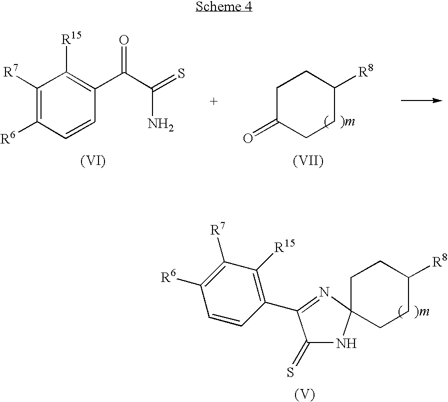

Thioamides of formula (VI) can be prepared from acylnitriles of formula (VIII) by treating with, for example hydrogen sulphide in the presence of an organic base, for example triethylamine in an inert solvent, for example diethyl ether at room temperature. Acylnitriles of formula (VIII) can be prepared from the appropriate acid chloride (IX) and a source of cyanide, conveniently copper (I) cyanide, at elevated temperatures, for example greater than 150° C. preferably in the absence of solvent.

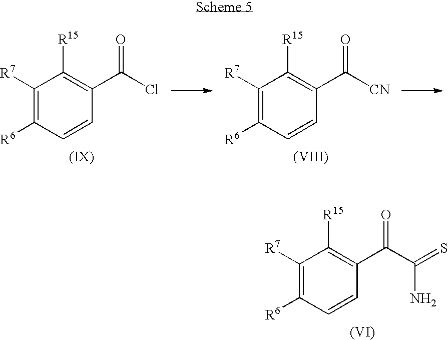

Alternatively, compounds of formula (II) can be synthesised as shown in Scheme 6.

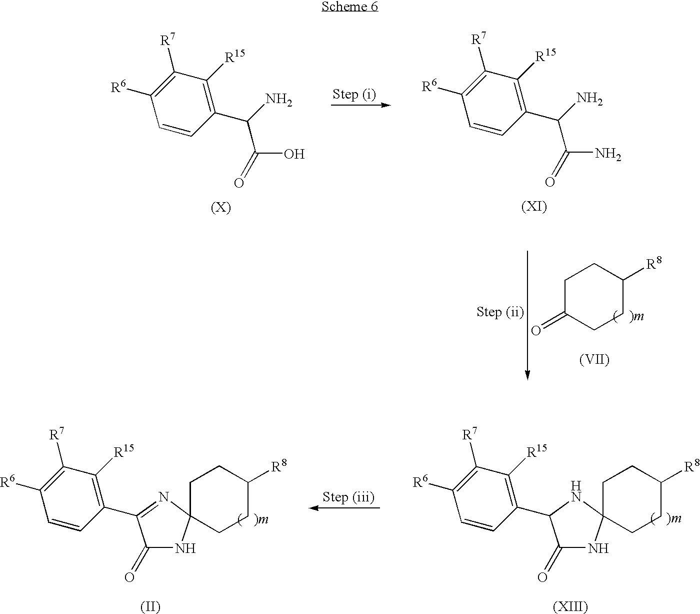

wherein R⁶, R⁷, R⁸ and R¹⁵ are as defined for formula (I).

The arylglycine of formula (X) can be converted, step (i), to the corresponding arylglycinamide of formula (XI) by standard methods, for example, by reaction of compounds of formula (X) with thionyl chloride or acetyl chloride in methanol, followed by subsequent reaction of the intermediate methyl ester hydrochloride with aqueous ammonia.

Arylglycinamides of formula (XI) can be converted to compounds of formula (XIII), step (ii), by condensation with ketones of formula (VII), for example, by heating in an inert solvent such as methanol, in the presence or absence of a catalyst such as H-Y zeolites.

Oxidation of compounds of formula (XIII), step (iii), to afford compounds of formula (II) can be achieved by methods known in the art, for example, by reaction with N-bromosuccinimide in an inert solvent, such as dichloromethane.

Compounds of formula (X) are known in the literature or can be prepared as shown in Scheme 7 wherein R⁶, R⁷ and R¹⁵ are as defined in formula (I), except when R⁷ is halo.

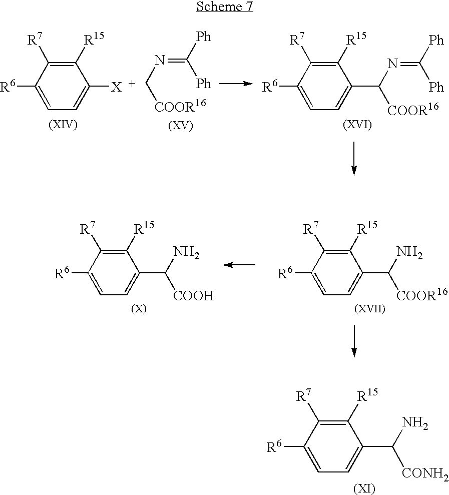

For example a compound of formula (XIV), where X is a halogen, can be treated with a N-(diphenylmethylidene)glycinate ester (XV), where R¹⁶ is lower alkyl such as methyl or ethyl, in the presence of a palladium catalyst such as bis(tri-t-butylphosphine)palladium (0) and a base such as potassium phosphate in a solvent such as toluene at elevated temperature to give a compound of formula (XVI). Mild acidic hydrolysis of the imine for example using dilute HCl acid at room temperature can afford the glycine ester (XVII), whereas the glycine (X) can be prepared by more extensive hydrolysis. Treatment of ester (XVII) with aqueous ammonia can give the glycinamide (XI).

Alternatively compounds of formula (II) can be prepared as shown in scheme 8 wherein R⁶, R⁷, R⁸ and R¹⁵ are as defined in formula (I) by application of palladium or copper mediated chemistry.

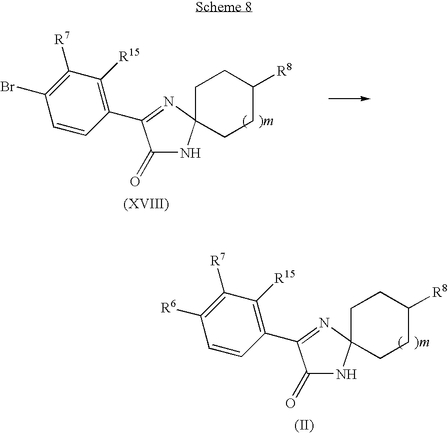

For example, treatment of a compound of structure (XVIII) with an appropriate palladium catalyst such as tetrakis(triphenylphosphine)palladium[0] or palladium acetate in conjunction with a phosphine ligand such as 1,3-(bis)triphenylphosphino)propane, a base such as sodium carbonate, triethylamine or diisopropylamine and a heteroaryl boronic acid or heteroaryl trialkyltin reagent may undergo palladium mediated coupling to give a compound of formula (II) where R⁶ is a carbon linked heteroaryl group. These reactions may be performed in a range of solvents including tetrahydrofuran, dimethylformamide, dioxan or toluene, or combinations of solvents, optionally in the presence of an ionic liquid such as 1-butyl-3-imidazolium tetrafluoroborate either at ambient or preferably elevated temperatures.

Alternatively, treatment of a compound of structure (XVIII) with an appropriate palladium catalyst such as tetrakis(triphenylphosphine)palladium[0] or palladium acetate in conjunction with a phosphine ligand such as 2,2′-bis(diphenylphosphino)-1,1′-binaphthalene (BINAP), a base such as cesium carbonate or potassium phosphate, and a 5 to 7 membered heterocyclic ring containing a secondary amine, such as piperidine or morpholine, may undergo palladium mediated coupling to give a compound of formula (II) where R⁶ is a nitrogen linked 5-7 membered heterocyclic group. These reactions may be performed in a range of solvents including tetrahydrofuran, dimethylformamide, dioxan or toluene, or combinations of solvents, optionally in the presence of an ionic liquid such as 1-butyl-3-imidazolium tetrafluoroborate either at ambient or preferably elevated temperatures.

Alternatively, treatment of a compound of structure (XVIII) with an appropriate copper catalyst such as copper (I) bromide or copper (I) iodide, in conjunction with a β-ketoester ligand such as ethyl 2-oxocyclohexanecarboxylate or diamine ligand such as trans-1,2-diaminocylohexane, a base such as cesium carbonate or potassium phosphate and a heteroaryl or 2-oxo substituted 5-7 membered heterocyclic ring containing a free NH, may undergo copper mediated coupling to give a compound of formula (II) where R⁶ is a nitrogen linked heteroaromatic or 2-oxo substituted 5-7 membered heterocyclic ring. These reactions may be performed in a range of solvents such as dimethyl sulphoxide, N,N-dimethylformamide, N-methylpyrrolidinone, acetonitrile or dioxan or combinations of solvents, optionally in the presence of an ionic liquid such as 1-butyl-3-imidazolium tetrafluoroborate either at ambient or preferably elevated temperatures.

Alternatively, compounds of formula (II) can be prepared as shown in scheme 9 wherein R⁷, R⁸ and R¹⁵ are as defined in formula (I) and R⁶ is a carbon linked heteroaryl group from intermediates such as where R⁶ is a carboxylic acid (XIX) or R⁶ is a cyano group (XX) using standard methods for preparation of heterocyclic systems such as those described in series such as Organic Syntheses, The Chemistry of Heterocycles or Comprehensive Heterocyclic Chemistry.

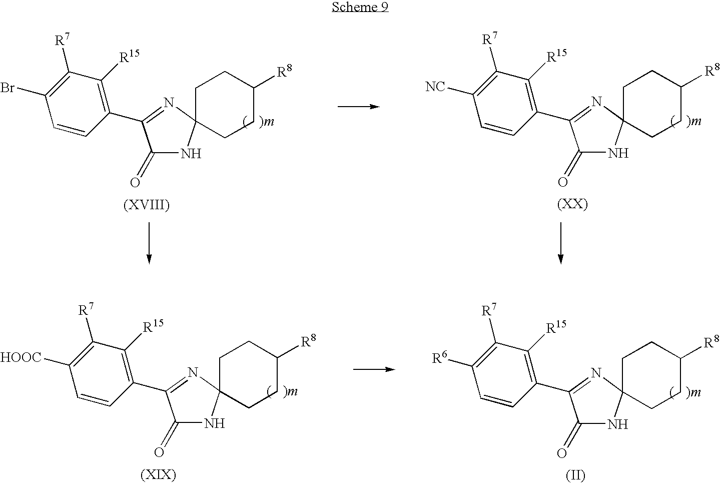

Nitrile (XX) can be prepared from (XVIII) by treatment with a cyanide source such as copper (I) cyanide, in a solvent such as N,N-dimethylformamide or N-methylpyrrolidinone at elevated temperature. Carboxylic acid (XIX) may be prepared by acidic hydrolysis of nitrile (XX) or directly from (XVIII) by treatment with 2 equivalents of alkyllithium at reduced temperature followed by addition of carbon dioxide, or through palladium mediated carbonylation methodology.

Compounds of formula (II) can also be converted to compounds of formula (I) as shown in Scheme 10.

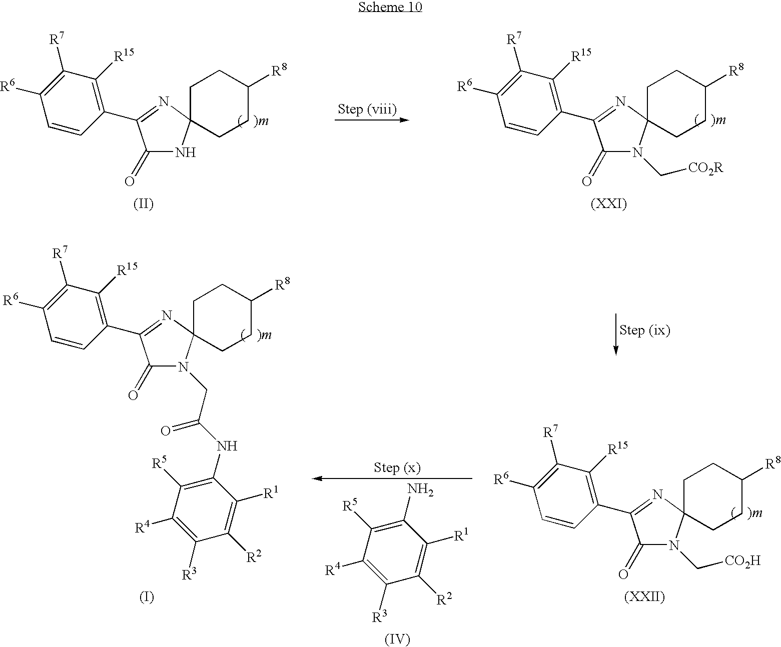

wherein R¹, R², R³, R⁴, R⁵, R⁶, R⁷, R⁸ and R¹⁵ are as defined for compounds of formula (I).

Compounds of formula (XXI) can be prepared using standard methods from compounds of formula (II), step (viii), for example, by reaction with an appropriate haloester in the presence of a base, such as sodium hydride or potassium carbonate, in a suitable inert solvent, such as dimethylformamide, at room temperature or elevated temperature as appropriate.

Removal of the ester group R from compounds of formula (XXI) to afford the acids of formula (XXII), step (ix), can be achieved by known methods, for example by use of a base, such as sodium hydroxide, in an inert solvent, such as aqueous methanol or aqueous ethanol, with or without heating as appropriate.

Compounds of formula (XXII) can be converted to compounds of formula (I), step (x), by reaction with an aniline of formula (IV) using a variety of methods known in the art. For example, the acylation step (x) can be achieved by reaction of the acid (XXII) with an aniline of formula (IV), in an inert solvent, such as dichloromethane in the presence of a coupling reagent, for example a diimide reagent such as N,N dicyclohexylcarbodiimide (DCC), N-(3-(dimethylamino)propyl)-N-ethylcarbodiimide hydrochloride (EDC), or O-(7-azabenzotriazol-1-yl)-1,1,3,3-tetramethyluronium hexafluoro phosphate (HATU). Alternatively, compounds of formula (XXII) are converted to compounds of formula (XXIII):

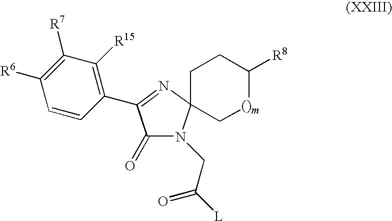

wherein R⁶, R⁷, R⁸ and R¹⁵ are as defined in formula (I) and L represents a suitable leaving group. Examples of leaving groups include halogen, OC(═O)alkyl, OC(═O)O-alkyl and OSO₂Me. L may be halogen and acylation in step (x) may be carried out in an inert solvent such as dichloromethane, in the presence of a base, such as triethylamine.

Within these schemes there is scope to convert a group R¹ into another group R¹ and similarly for groups R², R³, R⁴, R⁵, R⁶, R⁷ and R¹⁵. For example, scheme 11, a compound of formula (I) where R⁶ is bromo may be converted to compounds of formula (I) wherein R⁶ is heteroaryl or a 5-7 membered heterocyclic ring using either palladium or copper mediated coupling using methods as indicated in Scheme 8. Alternatively a heterocycle may be constructed from a compound of formula (I) where R⁶ is bromo via a carboxylic acid or cyano intermediate using procedures as indicated in Scheme 9.

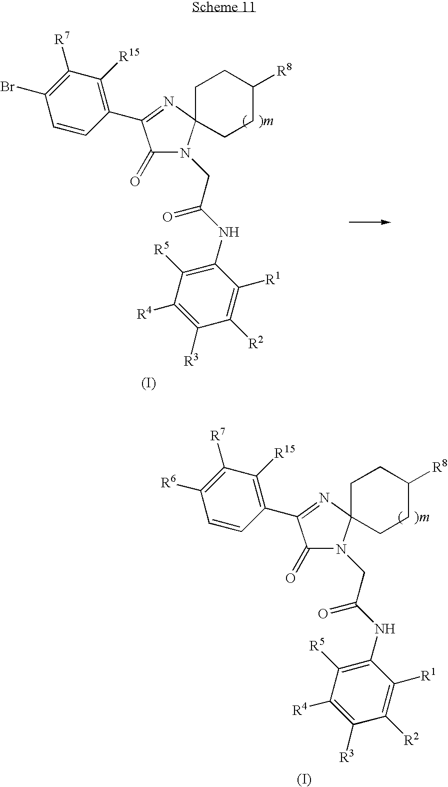

Salts may be prepared conventionally by reaction with the appropriate acid or acid derivative.

The affinities of the compounds of this invention for the GlyT1 transporter can be determined by the following assay.

HEK293 cells expressing the Glycine (Type 1) transporter were grown in cell culture medium [DMEM/NUT mix F12 containing 2 mM L-Glutamine, 0.8 mg/mL G418 and 10% heat inactivated fetal calf serum] at 37° C. and 5% CO₂. Cells grown to 70-80% confluency in T175 flasks were harvested and resuspended at 4×10⁵ cells/mL in assay buffer [140 mM NaCl, 5.4 mM KCl, 1.8 mM CaCl₂, 0.8 mM MgSO₄, 20 mM HEPES, 5 mM glucose and 5 mM alanine, pH 7.4]. Compounds were serially diluted 2.5-fold in DMSO from a top concentration of 2.5 mM with each compound giving a 11 data point dose-response. 100 nL of compound at each concentration was added to the assay plate. An equal volume of Leadseeker™ WGA SPA beads (12.5 mg/ml suspended in assay buffer) was added to the cell suspension and 5 μL of the cell/bead suspension transferred to each well of a 384-well white solid bottom plate (1,000 cells/well) containing 100 nL of test compounds. Substrate (5 μL) was added to each well [1:100 dilution of [³H]-glycine stock in assay buffer containing 2.5 μM glycine). Final DMSO concentration was 1% v/v. Data was collected using a Perkin Elmer Viewlux. pIC₅₀ values were determined using ActivityBase.

Compounds are considered to have activity at the the GlyT1 transporter if they have a pIC₅₀ of 5.0 or above in the above assay. The example compounds below and the individually named compounds above were found to have an pIC₅₀ at the GlyT1 transporter of, on average, greater than or equal to 5.6. In the assay, the compounds of the present invention were not necessarily from the same batch described below. A test compound from one batch may have been combined with other batch(es) for the assay(s).

The compounds of the present invention inhibit the GlyT1 transporter, as measured by the assay above. Such compounds are therefore of potential utility for the treatment of certain neurological and neuropsychiatric disorders. The compounds may selectively inhibit the GlyT1 transporter over the GlyT2 transporter. Some compounds of the invention may have mixed GlyT1/GlyT2 activity.

In one embodiment, the disorder to be treated by the use or method as hereinbefore described is a psychosis, including schizophrenia, dementia and attention deficit disorders. In one embodiment, the disorder is schizophrenia.

Within the context of the present invention, the terms used herein are classified in the Diagnostic and Statistical Manual of Mental Disorders, 4^{th} Edition, published by the American Psychiatric Association (DSM-IV) and/or the International Classification of Diseases, 10^{th} Edition (ICD-10). Treatment of the various subtypes of the disorders mentioned herein using the compounds of the invention are contemplated as part of the present invention. Numbers in brackets after the listed diseases below refer to the classification code in DSM-IV.

In particular, the compounds of the invention be of use in the treatment of schizophrenia including the subtypes Paranoid Type (295.30), Disorganised Type (295.10), Catatonic Type (295.20), Undifferentiated Type (295.90) and Residual Type (295.60); Schizophreniform Disorder (295.40); Schizoaffective Disorder (295.70) including the subtypes Bipolar Type and Depressive Type; Delusional Disorder (297.1) including the subtypes Erotomanic Type, Grandiose Type, Jealous Type, Persecutory Type, Somatic Type, Mixed Type and Unspecified Type; Brief Psychotic Disorder (298.8); Shared Psychotic Disorder (297.3); Psychotic Disorder Due to a General Medical Condition including the subtypes With Delusions and With Hallucinations; Substance-Induced Psychotic Disorder including the subtypes With Delusions (293.81) and With Hallucinations (293.82); and Psychotic Disorder Not Otherwise Specified (298.9).

The compounds of the invention may be also of use in the treatment of mood disorders including Major Depressive Episode, Manic Episode, Mixed Episode and Hypomanic Episode; Depressive Disorders including Major Depressive Disorder, Dysthymic Disorder (300.4), Depressive Disorder Not Otherwise Specified (311); Bipolar Disorders including Bipolar I Disorder, Bipolar II Disorder (Recurrent Major Depressive Episodes with Hypomanic Episodes) (296.89), Cyclothymic Disorder (301.13) and Bipolar Disorder Not Otherwise Specified (296.80); Other Mood Disorders including Mood Disorder Due to a General Medical Condition (293.83) which includes the subtypes With Depressive Features, With Major Depressive-like Episode, With Manic Features and With Mixed Features), Substance-Induced Mood Disorder (including the subtypes With Depressive Features, With Manic Features and With Mixed Features) and Mood Disorder Not Otherwise Specified (296.90).

The compounds of the invention may also be of use in the treatment of anxiety disorders including Panic Attack, Agoraphobia, Panic Disorder, Agoraphobia Without History of Panic Disorder (300.22), Specific Phobia (300.29) including the subtypes Animal Type, Natural Environment Type, Blood-Injection-Injury Type, Situational Type and Other Type), Social Phobia (300.23), Obsessive-Compulsive Disorder (300.3), Posttraumatic Stress Disorder (309.81), Acute Stress Disorder (308.3), Generalized Anxiety Disorder (300.02), Anxiety Disorder Due to a General Medical Condition (293.84), Substance-Induced Anxiety Disorder and Anxiety Disorder Not Otherwise Specified (300.00).

The compounds of the invention may also be of use in the treatment of substance-related disorders including Substance Use Disorders such as Substance Dependence and Substance Abuse; Substance-Induced Disorders such as Substance Intoxication, Substance Withdrawal, Substance-Induced Delirium, Substance-Induced Persisting Dementia, Substance-Induced Persisting Amnestic Disorder, Substance-Induced Psychotic Disorder, Substance-Induced Mood Disorder, Substance-Induced Anxiety Disorder, Substance-Induced Sexual Dysfunction, Substance-Induced Sleep Disorder and Hallucinogen Persisting Perception Disorder (Flashbacks); Alcohol-Related Disorders such as Alcohol Dependence (303.90), Alcohol Abuse (305.00), Alcohol Intoxication (303.00), Alcohol Withdrawal (291.81), Alcohol Intoxication Delirium, Alcohol Withdrawal Delirium, Alcohol-Induced Persisting Dementia, Alcohol-Induced Persisting Amnestic Disorder, Alcohol-Induced Psychotic Disorder, Alcohol-Induced Mood Disorder, Alcohol-Induced Anxiety Disorder, Alcohol-Induced Sexual Dysfunction, Alcohol-Induced Sleep Disorder and Alcohol-Related Disorder Not Otherwise Specified (291.9); Amphetamine (or Amphetamine-Like)-Related Disorders such as Amphetamine Dependence (304.40), Amphetamine Abuse (305.70), Amphetamine Intoxication (292.89), Amphetamine Withdrawal (292.0), Amphetamine Intoxication Delirium, Amphetamine Induced Psychotic Disorder, Amphetamine-Induced Mood Disorder, Amphetamine-Induced Anxiety Disorder, Amphetamine-Induced Sexual Dysfunction, Amphetamine-Induced Sleep Disorder and Amphetamine-Related Disorder Not Otherwise Specified (292.9); Caffeine Related Disorders such as Caffeine Intoxication (305.90), Caffeine-Induced Anxiety Disorder, Caffeine-Induced Sleep Disorder and Caffeine-Related Disorder Not Otherwise Specified (292.9); Cannabis-Related Disorders such as Cannabis Dependence (304.30), Cannabis Abuse (305.20), Cannabis Intoxication (292.89), Cannabis Intoxication Delirium, Cannabis-Induced Psychotic Disorder, Cannabis-Induced Anxiety Disorder and Cannabis-Related Disorder Not Otherwise Specified (292.9); Cocaine-Related Disorders such as Cocaine Dependence (304.20), Cocaine Abuse (305.60), Cocaine Intoxication (292.89), Cocaine Withdrawal (292.0), Cocaine Intoxication Delirium, Cocaine-Induced Psychotic Disorder, Cocaine-Induced Mood Disorder, Cocaine-Induced Anxiety Disorder, Cocaine-Induced Sexual Dysfunction, Cocaine-Induced Sleep Disorder and Cocaine-Related Disorder Not Otherwise Specified (292.9); Hallucinogen-Related Disorders such as Hallucinogen Dependence (304.50), Hallucinogen Abuse (305.30), Hallucinogen Intoxication (292.89), Hallucinogen Persisting Perception Disorder (Flashbacks) (292.89), Hallucinogen Intoxication Delirium, Hallucinogen-Induced Psychotic Disorder, Hallucinogen-Induced Mood Disorder, Hallucinogen-Induced Anxiety Disorder and Hallucinogen-Related Disorder Not Otherwise Specified (292.9); Inhalant-Related Disorders such as Inhalant Dependence (304.60), Inhalant Abuse (305.90), Inhalant Intoxication (292.89), Inhalant Intoxication Delirium, Inhalant-Induced Persisting Dementia, Inhalant-Induced Psychotic Disorder, Inhalant-Induced Mood Disorder, Inhalant-Induced Anxiety Disorder and Inhalant-Related Disorder Not Otherwise Specified (292.9); Nicotine-Related Disorders such as Nicotine Dependence (305.1), Nicotine Withdrawal (292.0) and Nicotine-Related Disorder Not Otherwise Specified (292.9); Opioid-Related Disorders such as Opioid Dependence (304.00), Opioid Abuse (305.50), Opioid Intoxication (292.89), Opioid Withdrawal (292.0), Opioid Intoxication Delirium, Opioid-Induced Psychotic Disorder, Opioid-Induced Mood Disorder, Opioid-Induced Sexual Dysfunction, Opioid-Induced Sleep Disorder and Opioid-Related Disorder Not Otherwise Specified (292.9); Phencyclidine (or Phencyclidine-Like)-Related Disorders such as Phencyclidine Dependence (304.60), Phencyclidine Abuse (305.90), Phencyclidine Intoxication (292.89), Phencyclidine Intoxication Delirium, Phencyclidine-Induced Psychotic Disorder, Phencyclidine-Induced Mood Disorder, Phencyclidine-Induced Anxiety Disorder and Phencyclidine-Related Disorder Not Otherwise Specified (292.9); Sedative-, Hypnotic-, or Anxiolytic-Related Disorders such as Sedative, Hypnotic, or Anxiolytic Dependence (304.10), Sedative, Hypnotic, or Anxiolytic Abuse (305.40), Sedative, Hypnotic, or Anxiolytic Intoxication (292.89), Sedative, Hypnotic, or Anxiolytic Withdrawal (292.0), Sedative, Hypnotic, or Anxiolytic Intoxication Delirium, Sedative, Hypnotic, or Anxiolytic Withdrawal Delirium, Sedative-, Hypnotic-, or Anxiolytic-Persisting Dementia, Sedative-, Hypnotic-, or Anxiolytic- Persisting Amnestic Disorder, Sedative-, Hypnotic-, or Anxiolytic-Induced Psychotic Disorder, Sedative-, Hypnotic-, or Anxiolytic-Induced Mood Disorder, Sedative-, Hypnotic-, or Anxiolytic-Induced Anxiety Disorder Sedative-, Hypnotic-, or Anxiolytic-Induced Sexual Dysfunction, Sedative-, Hypnotic-, or Anxiolytic-Induced Sleep Disorder and Sedative-, Hypnotic-, or Anxiolytic-Related Disorder Not Otherwise Specified (292.9); Polysubstance-Related Disorder such as Polysubstance Dependence (304.80); and Other (or Unknown) Substance-Related Disorders such as Anabolic Steroids, Nitrate Inhalants and Nitrous Oxide.

The compounds of the invention may also be of use in the treatment of sleep disorders including primary sleep disorders such as Dyssomnias such as Primary Insomnia (307.42), Primary Hypersomnia (307.44), Narcolepsy (347), Breathing-Related Sleep Disorders (780.59), Circadian Rhythm Sleep Disorder (307.45) and Dyssomnia Not Otherwise Specified (307.47); primary sleep disorders such as Parasomnias such as Nightmare Disorder (307.47), Sleep Terror Disorder (307.46), Sleepwalking Disorder (307.46) and Parasomnia Not Otherwise Specified (307.47); Sleep Disorders Related to Another Mental Disorder such as Insomnia Related to Another Mental Disorder (307.42) and Hypersomnia Related to Another Mental Disorder (307.44); Sleep Disorder Due to a General Medical Condition; and Substance-Induced Sleep Disorder including the subtypes Insomnia Type, Hypersomnia Type, Parasomnia Type and Mixed Type.

The compounds of the invention may also be of use in the treatment of eating disorders such as Anorexia Nervosa (307.1) including the subtypes Restricting Type and Binge-Eating/Purging Type; Bulimia Nervosa (307.51) including the subtypes Purging Type and Nonpurging Type; Obesity; Compulsive Eating Disorder; and Eating Disorder Not Otherwise Specified (307.50).

The compounds of the invention may also be of use in the treatment of Autistic Disorder (299.00); Attention-Deficit/Hyperactivity Disorder including the subtypes Attention-Deficit/Hyperactivity Disorder Combined Type (314.01), Attention-Deficit/Hyperactivity Disorder Predominantly Inattentive Type (314.00), Attention-Deficit/Hyperactivity Disorder Hyperactive-Impulse Type (314.01) and Attention-Deficit/Hyperactivity Disorder Not Otherwise Specified (314.9); Hyperkinetic Disorder; Disruptive Behaviour Disorders such as Conduct Disorder including the subtypes childhood-onset type (321.81), Adolescent-Onset Type (312.82) and Unspecified Onset (312.89), Oppositional Defiant Disorder (313.81) and Disruptive Behaviour Disorder Not Otherwise Specified; and Tic Disorders such as Tourette's Disorder (307.23).

The compounds of the invention may also be of use in the treatment of Personality Disorders including the subtypes Paranoid Personality Disorder (301.0), Schizoid Personality Disorder (301.20), Schizotypal Personality Disorder (301,22), Antisocial Personality Disorder (301.7), Borderline Personality Disorder (301,83), Histrionic Personality Disorder (301.50), Narcissistic Personality Disorder (301,81), Avoidant Personality Disorder (301.82), Dependent Personality Disorder (301.6), Obsessive-Compulsive Personality Disorder (301.4) and Personality Disorder Not Otherwise Specified (301.9).

The compounds of the invention may also be of use in the treatment of cognitive impairment. Within the context of the present invention, the term cognitive impairment includes for example the treatment of impairment of cognitive functions including attention, orientation, learning disorders, memory (i.e. memory disorders, amnesia, amnesic disorders, transient global amnesia syndrome and age-associated memory impairment) and language function; cognitive impairment as a result of stroke, Alzheimer's disease, Huntington's disease, Pick disease, Aids-related dementia or other dementia states such as Multiinfarct dementia, alcoholic dementia, hypotiroidism-related dementia, and dementia associated to other degenerative disorders such as cerebellar atrophy and amyotropic lateral sclerosis; other acute or sub-acute conditions that may cause cognitive decline such as delirium or depression (pseudodementia states) trauma, head trauma, age related cognitive decline, stroke, neurodegeneration, drug-induced states, neurotoxic agents, mild cognitive impairment, age related cognitive impairment, autism related cognitive impairment, Down's syndrome, cognitive deficit related to psychosis, and post-electroconvulsive treatment related cognitive disorders; and dyskinetic disorders such as Parkinson's disease, neuroleptic-induced parkinsonism, and tardive dyskinesias.

The compounds of the present invention may also be of use for the treatment of cognition impairment which arises in association or as a result of other diseases such as schizophrenia, bipolar disorder, depression, other psychiatric disorders and psychotic conditions associated with cognitive impairment.

The compounds of the invention may also be of use in the treatment of sexual dysfunctions including Sexual Desire Disorders such as Hypoactive Sexual Desire Disorder (302.71), and Sexual Aversion Disorder (302.79); sexual arousal disorders such as Female Sexual Arousal Disorder (302.72) and Male Erectile Disorder (302.72); orgasmic disorders such as Female Orgasmic Disorder (302.73), Male Orgasmic Disorder (302.74) and Premature Ejaculation (302.75); sexual pain disorder such as Dyspareunia (302.76) and Vaginismus (306.51); Sexual Dysfunction Not Otherwise Specified (302.70); paraphilias such as Exhibitionism (302.4), Fetishism (302.81), Frotteurism (302.89), Pedophilia (302.2), Sexual Masochism (302.83), Sexual Sadism (302.84), Transvestic Fetishism (302.3), Voyeurism (302.82) and Paraphilia Not Otherwise Specified (302.9); gender identity disorders such as Gender Identity Disorder in Children (302.6) and Gender Identity Disorder in Adolescents or Adults (302.85); and Sexual Disorder Not Otherwise Specified (302.9).

The compounds of the invention may also be of use as anticonvulsants. The compounds of the invention are thus useful in the treatment of convulsions in mammals, and particularly epilepsy in humans. “Epilepsy” is intended to include the following seizures: simple partial seizures, complex partial seizures, secondary generalised seizures, generalised seizures including absence seizures, myoclonic seizures, clonic seizures, tonic seizures, tonic clonic seizures and atonic seizures. The invention also provides a method of treating convulsions, which comprises administering to a mammal in need thereof an effective amount of a compound of the invention as hereinbefore described or a salt thereof. Treatment of epilepsy may be carried out by the administration of a non-toxic anticonvulsant effective amount of a compound of the formula (I) or a salt thereof.

The compounds of the invention may also be of use in the treatment of neuropathic pain, for example in diabetic neuropathy, sciatica, non-specific lower back pain, multiple sclerosis pain, fibromyalgia, HIV-related neuropathy, neuralgia such as post-herpetic neuralgia and trigeminal neuralgia and pain resulting from physical trauma, amputation, cancer, toxins or chronic inflammatory conditions.

As used herein, the terms “treatment” and “treating” refer to the alleviation and/or cure of established symptoms as well as prophylaxis.

The invention thus provides compounds of formula (I) and pharmaceutically acceptable salts thereof for use in therapy.

The invention also provides compounds of formula (I) and pharmaceutically acceptable salts thereof for use in the treatment of a disorder wherein inhibition of GlyT1 would be beneficial.

In a further aspect of the present invention, there is provided a method of treating a disorder wherein inhibition of GlyT1 would be beneficial comprising administering an effective amount of a compound of formula (I) or a pharmaceutically acceptable salt thereof.

As used herein, the term “effective amount” means that amount of a drug or pharmaceutical agent that will elicit the biological or medical response of a tissue, system, animal or human that is being sought, for instance, by a researcher or clinician.

In a further aspect of the present invention there is provided the use of a compound of formula (I) or a pharmaceutically acceptable salt thereof in the manufacture of a medicament for use in the treatment of a disorder wherein inhibition of GlyT1 would be beneficial.

In order to use a compound of the present invention in therapy, it will normally be formulated into a pharmaceutical composition in accordance with standard pharmaceutical practice. The present invention also provides a pharmaceutical composition, which comprises a compound of formula (I) or a pharmaceutically acceptable salt thereof and at least one pharmaceutically acceptable excipient.

In a further aspect, the present invention provides a process for preparing a pharmaceutical composition, the process comprising mixing a compound of formula (I) or a pharmaceutically acceptable salt thereof and at least one pharmaceutically acceptable excipient.

A pharmaceutical composition of the invention is usually adapted for oral, sub-lingual, buccal, parenteral (for example, subcutaneous, intramuscular, or intravenous), rectal, topical and intranasal administration and in forms suitable for administration by inhalation or insufflation (either through the mouth or nose). The most suitable means of administration for a particular patient will depend on the nature and severity of the conditions being treated and on the nature of the active compound. In one embodiment, oral administration is provided.

Compositions suitable for oral administration may be provided as discrete units, such as tablets, capsules, cachets, or lozenges, each containing a predetermined amount of the active compound; as powders or granules; as solutions or suspensions in aqueous or non-aqueous liquids; or as oil-in-water or water-in-oil emulsions.

Compositions suitable for sublingual or buccal administration include lozenges comprising the active compound and, typically, a flavoured base, such as sugar and acacia or tragacanth and pastilles comprising the active compound in an inert base, such as gelatin and glycerin or sucrose and acacia.

Compositions suitable for parenteral administration typically comprise sterile aqueous solutions containing a predetermined concentration of the active compound; the solution may be isotonic with the blood of the intended recipient. Such solutions may be administered intravenously or by subcutaneous or intramuscular injection.

Compositions suitable for rectal administration may be provided as unit-dose suppositories comprising the active ingredient and one or more solid carriers forming the suppository base, for example, cocoa butter.

Compositions suitable for topical or intranasal application include ointments, creams, lotions, pastes, gels, sprays, aerosols and oils. Suitable carriers for such compositions include petroleum jelly, lanolin, polyethylene glycols, alcohols, and combinations thereof.

The compositions of the invention may be prepared by any suitable method, typically by uniformly and intimately admixing the active compound(s) with liquids or finely divided solid carriers, or both, in the required proportions and then, if necessary, shaping the resulting mixture into the desired shape.

For example, a tablet may be prepared by compressing an intimate mixture comprising a powder or granules of the active ingredient and one or more optional ingredients, such as a binder, lubricant, inert diluent, or surface active dispersing agent, or by moulding an intimate mixture of powdered active ingredient and inert liquid diluent.

Aqueous solutions for parenteral administration are typically prepared by dissolving the active compound in sufficient water to give the desired concentration and then rendering the resulting solution sterile and isotonic.

It will be appreciated that the precise dose administered will depend on the age and condition of the patient and the frequency and route of administration and will be at the ultimate discretion of the attendant physician. The compound may be administered in single or divided doses and may be administered one or more times, for example 1 to 4 times per day.

A proposed dose of the active ingredient for use according to the invention for oral, sub-lingual, parenteral, buccal, rectal, intranasal or topical administration to a human (of approximately 70 kg bodyweight) for the treatment of neurological and neuropsychiatric disorders mediated by a GlyT1 inhibitor, including schizophrenia, may be about 0.1 to about 1000 mg, for example about 0.5 mg to about 1000 mg, or about 1 mg to about 1000 mg, or about 5 mg to about 500 mg, or about 10 mg to about 100 mg of the active ingredient per unit dose, which could be administered, for example, 1 to 4 times per day.

The compounds of formula (I) and their pharmaceutically acceptable salts thereof may also be suitable for combination with other therapeutic agents, such as typical and atypical antipsychotics. Thus, the present invention also provides:
i) a combination comprising a compound of formula (I) or a pharmaceutically acceptable salt thereof with one or more further therapeutic agents such an one or more antipsychotic;
ii) a pharmaceutical composition comprising a combination product as defined in i) above and at least one pharmaceutically acceptable excipient;
iii) the use of a combination as defined in i) above in the manufacture of a medicament for treating or a disorder wherein inhibition of GlyT1 would be beneficial;
iv) a combination as defined in i) above for use in treating or a disorder wherein inhibition of GlyT1 would be beneficial;
v) a kit-of-parts for use in the treatment of a psychotic disorder comprising a first dosage form comprising a compound of the invention and one or more further dosage forms each comprising a antipsychotic agent for simultaneous therapeutic administration.
vi) a combination as defined in i) above for use in therapy;
vii) a method of treating a disorder wherein inhibition of GlyT1 would be beneficial comprising administering an effective amount of a combination as defined in i) above.

The combination therapies of the invention may be administered adjunctively. By adjunctive administration is meant the coterminous or overlapping administration of each of the components in the form of separate pharmaceutical compositions or devices. This regime of therapeutic administration of two or more therapeutic agents is referred to generally by those skilled in the art and herein as adjunctive therapeutic administration; it is also known as add-on therapeutic administration. Any and all treatment regimes in which a patient receives separate but coterminous or overlapping therapeutic administration of the compounds of formula (I) or a pharmaceutically acceptable salt thereof and at least one antipsychotic agent are within the scope of the current invention. In one embodiment of adjunctive therapeutic administration as described herein, a patient is typically stabilised on a therapeutic administration of one or more of the of the components for a period of time and then receives administration of another component. Within the scope of this invention, the compounds of formula (I) or a pharmaceutically acceptable salt thereof may be administered as adjunctive therapeutic treatment to patients who are receiving administration of at least one antipsychotic agent, but the scope of the invention also includes the adjunctive therapeutic administration of at least one antipsychotic agent to patients who are receiving administration of compounds of formula (I) or a pharmaceutically acceptable salt thereof.

The combination therapies of the invention may also be administered simultaneously. By simultaneous administration is meant a treatment regime wherein the individual components are administered together, either in the form of a single pharmaceutical composition or device comprising or containing both components, or as separate compositions or devices, each comprising one of the components, administered simultaneously. Such combinations of the separate individual components for simultaneous combination may be provided in the form of a kit-of-parts.

In a further aspect therefore, the invention provides a method of treatment of a psychotic disorder by adjunctive therapeutic administration of compounds of formula (I) or a pharmaceutically acceptable salt thereof to a patient receiving therapeutic administration of at least one antipsychotic agent. In a further aspect, the invention provides the use of compounds of formula (I) or a pharmaceutically acceptable salt thereof in the manufacture of a medicament for adjunctive therapeutic administration for the treatment of a psychotic disorder in a patient receiving therapeutic administration of at least one antipsychotic agent. The invention further provides compounds of formula (I) or a pharmaceutically acceptable salt thereof for use for adjunctive therapeutic administration for the treatment of a psychotic disorder in a patient receiving therapeutic administration of at least one antipsychotic agent.

In a further aspect, the invention provides a method of treatment of a psychotic disorder by adjunctive therapeutic administration of at least one antipsychotic agent to a patient receiving therapeutic administration of compounds of formula (I) or a pharmaceutically acceptable salt thereof. In a further aspect, the invention provides the use of at least one antipsychotic agent in the manufacture of a medicament for adjunctive therapeutic administration for the treatment of a psychotic disorder in a patient receiving therapeutic administration of compounds of formula (I) or a pharmaceutically acceptable salt thereof. The invention further provides at least one antipsychotic agent for adjunctive therapeutic administration for the treatment of a psychotic disorder in a patient receiving therapeutic administration of compounds of formula (I) or pharmaceutically acceptable a salt thereof.

In a further aspect, the invention provides a method of treatment of a psychotic disorder by simultaneous therapeutic administration of compounds of formula (I) or a pharmaceutically acceptable salt thereof in combination with at least one antipsychotic agent. The invention further provides the use of a combination of compounds of formula (I) or a salt thereof and at least one antipsychotic agent in the manufacture of a medicament for simultaneous therapeutic administration in the treatment of a psychotic disorder. The invention further provides the use of compounds of formula (I) or a pharmaceutically acceptable salt thereof in the manufacture of a medicament for simultaneous therapeutic administration with at least one antipsychotic agent in the treatment of a psychotic disorder. The invention further provides compounds of formula (I) or a pharmaceutically acceptable salt thereof for use for simultaneous therapeutic administration with at least one antipsychotic agent in the treatment of a psychotic disorder. The invention further provides the use of at least one antipsychotic agent in the manufacture of a medicament for simultaneous therapeutic administration with compounds of formula (I) or a pharmaceutically acceptable salt thereof in the treatment of a psychotic disorder.

In further aspects, the invention provides a method of treatment of a psychotic disorder by simultaneous therapeutic administration of a pharmaceutical composition comprising compounds of formula (I) or a pharmaceutically acceptable salt thereof and at least one mood stabilising or antimanic agent, a pharmaceutical composition comprising compounds of formula (I) or a pharmaceutically acceptable salt thereof and at least one mood stabilising or antimanic agent, the use of a pharmaceutical composition comprising compounds of formula (I) or a pharmaceutically acceptable salt thereof and at least one mood stabilising or antimanic agent in the manufacture of a medicament for the treatment of a psychotic disorder, and a pharmaceutical composition comprising compounds of formula (I) or a pharmaceutically acceptable salt thereof and at least one mood stabilising or antimanic agent for use in the treatment of a psychotic disorder.

Examples of antipsychotic drugs that are useful in the present invention include, but are not limited to: butyrophenones, such as haloperidol, pimozide, and droperidol; phenothiazines, such as chlorpromazine, thioridazine, mesoridazine, trifluoperazine, perphenazine, fluphenazine, thiflupromazine, prochlorperazine, and acetophenazine; thioxanthenes, such as thiothixene and chlorprothixene; thienobenzodiazepines; dibenzodiazepines; benzisoxazoles; dibenzothiazepines; imidazolidinones; benziso-thiazolyl-piperazines; triazine such as lamotrigine; dibenzoxazepines, such as loxapine; dihydroindolones, such as molindone; aripiprazole; and derivatives thereof that have antipsychotic activity.

Examples of tradenames and suppliers of selected antipsychotic drugs are as follows: clozapine (available under the tradename CLOZARIL®, from Mylan, Zenith Goldline, UDL, Novartis); olanzapine (available under the tradename ZYPREX®, from Lilly; ziprasidone (available under the tradename GEODON®, from Pfizer); risperidone (available under the tradename RISPERDAL®, from Janssen); quetiapine fumarate (available under the tradename SEROQUEL®, from AstraZeneca); haloperidol (available under the tradename HALDOL®, from Ortho-McNeil); chlorpromazine (available under the tradename THORAZINE®, from SmithKline Beecham (GSK); fluphenazine (available under the tradename PROLIXIN®, from Apothecon, Copley, Schering, Teva, and American Pharmaceutical Partners, Pasadena); thiothixene (available under the tradename NAVANE®;, from Pfizer); trifluoperazine (10-[3-(4-methyl-1-piperazinyl)propyl]-2-(trifluoromethyl)phenothiazine dihydrochloride, available under the tradename STELAZINE®, from Smith Klein Beckman; perphenazine (available under the tradename TRILAFON®; from Schering); thioridazine (available under the tradename MELLARIL®; from Novartis, Roxane, HiTech, Teva, and Alpharma) ; molindone (available under the tradename MOBAN®, from Endo); and loxapine (available under the tradename LOXITANE®; from Watson). Furthermore, benperidol (Glianimon®), perazine (Taxilan®) or melperone (Eunerpan®)) may be used. Other antipsychotic drugs include promazine (available under the tradename SPARINE®), triflurpromazine (available under the tradename VESPRIN®), chlorprothixene (available under the tradename TARACTAN®), droperidol (available under the tradename INAPSINE®), acetophenazine (available under the tradename TINDAL®;), prochlorperazine (available under the tradename COMPAZINE®), methotrimeprazine (available under the tradename NOZINAN®), pipotiazine (available under the tradename PIPOTRIL®), ziprasidone, and hoperidone.

It will be appreciated by those skilled in the art that the compounds according to the invention may advantageously be used in conjunction with one or more other therapeutic agents, for instance, antidepressant agents such as 5HT3 antagonists, serotonin agonists, NK-1 antagonists, selective serotonin reuptake inhibitors (SSRI), noradrenaline re-uptake inhibitors (SNRI), tricyclic antidepressants, dopaminergic antidepressants, H3 antagonists, 5HT1A antagonists, 5HT1B antagonists, 5HT1D antagonists, D1 agonists, M1 agonists and/or anticonvulsant agents, as well as cognitive enhancers.

Suitable 5HT3 antagonists which may be used in combination of the compounds of the inventions include for example ondansetron, granisetron, metoclopramide.

Suitable serotonin agonists which may be used in combination with the compounds of the invention include sumatriptan, rauwolscine, yohimbine, metoclopramide.

Suitable SSRIs which may be used in combination with the compounds of the invention include fluoxetine, citalopram, femoxetine, fluvoxamine, paroxetine, indalpine, sertraline, zimeldine.

Suitable SNRIs which may be used in combination with the compounds of the invention include venlafaxine and reboxetine.

Suitable tricyclic antidepressants which may be used in combination with a compound of the invention include imipramine, amitriptiline, chlomipramine and nortriptiline.

Suitable dopaminergic antidepressants which may be used in combination with a compound of the invention include bupropion and amineptine.

Suitable anticonvulsant agents which may be used in combination of the compounds of the invention include for example divalproex, carbamazepine and diazepam.

The invention is further illustrated by the following non-limiting examples.

Starting materials, reagents and solvents were obtained from commercial suppliers and used without further purification unless otherwise stated. H-Y Zeolites was purchased from Zeolyst International as product CBV400. Chromatography was carried out using pre-packed Isolute Flash™ or Biotage™ silica-gel columns as the stationary phase and analytical grade solvents as the eluent unless otherwise stated. SCX cartridge refers to Varian Bond Elut™ MEGA BE_SCX (strong cationic exchange) cartridges. SCX-2 refers to IST Isolute SCX-2 cartridges. Aminopropyl-silica cartridge refers to Biotage Isolute™ SPE cartridges (part no. 470-1000-F). Phase separation cartridge or hydrophobic frit refers to Biotage Isolute Phase Separator.

NMR spectra were obtained at 298K, 303.2K or 300K, at the frequency stated using either a Bruker™ DPX400 or AV400 machine and run as a dilute solution of CDCl₃ unless otherwise stated. All NMR spectra were referenced to tetramethylsilane (TMS δ_{H} 0, δ_{C} 0). All coupling constants are reported in hertz (Hz), and multiplicities are labelled s (singlet), bs (broad singlet), d (doublet), t (triplet), q (quartet), dd (doublet of doublets), dt (doublet of triplets), td (triplet of doublets), ddd (double-double-doublet) and m (multiplet).

All quoted retention times are as measured using LC/MS (Liquid Chromatography/Mass Spectrometry). Where appropriate, these retention times were used as a guide for purification using mass-directed auto-purification (MDAP), which refers to purification by HPLC, wherein fraction collection is triggered by detection of the programmed mass ion for the compound of interest.

Total ion current traces were obtained for electrospray positive and negative ionisation (ES+/ES−) and/or atmospheric pressure chemical positive and negative ionisation (AP+/AP−).

Where reactions are described as having been carried out in a similar manner to earlier, more completely described reactions, the general reaction conditions used were essentially the same. Work up conditions used were of the types standard in the art, but may have been adapted from one reaction to another. The starting material may not necessarily have been prepared from the batch referred to. Unless otherwise stated, all compounds with chiral centre(s) are racemic. Compounds synthesised may have various purities ranging from for example 85% to 98%. However, calculations of number of moles and yield are generally not adjusted for this. All reactions were either carried out under argon or may be carried out under argon, unless otherwise stated.

Abbreviations:
LC/MS liquid chromatography/mass spectrometry
NMR nuclear magnetic resonance
MeOH methanol
EtOAc ethyl acetate
HCl hydrochloric acid
Et₂O diethylether
MDAP mass directed auto-purification system
THF tetrahydrofuran
DCM/MDC dichloromethane/methylene dichloride
DMF dimethylformamide
DMSO dimethyl sulfoxide
MeOH methanol
DIPEA diisopropylethylamine
HATU O-(7-azabenzotriazol-1-yl)-N,N,N′,N′-tetramethyluronium hexafluorophosphate
g grams
h/hr/hrs hour(s)
iPrOH isopropyl alcohol
ml millilitres
mmol millimoles

Analytical LC/MS hromatography Conditions:
Column: Waters Atlantis 50 mm×4.6 mm, 3 um particle size or Sunfire C-18 30 mm×4.6 mm, 3.5 um particle size
Mobile phase: A: 0.05% Formic acid+Water B: Acetonitrile +0.05% Formic acid
Gradient: 5-min runtime: 3% B to 97% B over 4 min
Flow rate: 3 ml/min
UV wavelength range: 220-330 nm
Temperature: 30° C.

Mass Directed Auto-Purification System Chromatography Conditions:

Mass-directed HPLC refers to methods where the material was purified by HPLC wherein fraction collection is triggered by detection of the programmed mass ion for the compound of interest.
Column: Waters Atlantis 19 mm×100 mm or 30 mm×100 mm, 5 um particle size or Sunfire C18 30 mm×150 mm, 5 um particle size
Mobile phase: A: 0.1% Formic acid+Water B: Acetonitrile+0.1% Formic acid
Flow rate: 20 or 40 ml/min

There are five methods used depending on the analytical retention time of the compound of interest. They have a 13.5-minute runtime, which comprises of a 10-minute gradient followed by a 3.5 minute column flush and re-equilibration step. (i) 1.0-1.5 mins=5-30% B; (ii) 1.5-2.2=15-55% B; (iii) 2.2-2.9=30-85% B; (iv) 2.9-3.6 mins=50-99% B, (v) 3.6-5.0 mins=80-99% B (in 6 minutes followed by 7.5 minutes flush and re-equilibration).

### Description 1. 2-Amino-2-(4-bromophenyl)acetamide

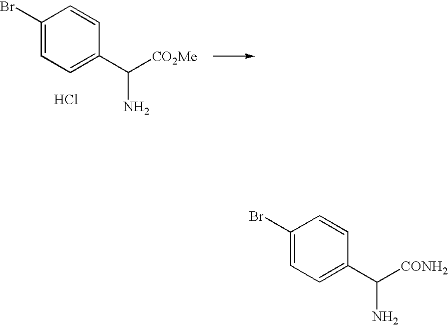

Methyl amino(4-bromophenyl)acetate hydrochloride (10 g, 41.0 mmol) in 0.88 ammonia (120 ml), was stirred at room temperature overnight. The white precipitate was collected by filtration and dried to give the title compound (4.95 g).

¹H NMR (CD₃OD) δ: 4.40 (1H, s), 4.88 (4H, m), 7.3 (2H, m), 7.5 (2H, m).

### Description 2. 3-(4-Bromophenyl)-1,4-diazaspiro[4.4]nonan-2-one

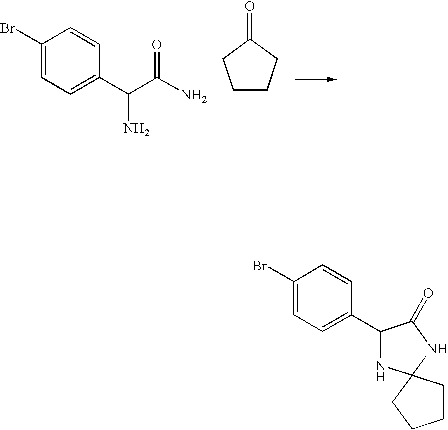

A mixture of cyclopentanone (0.73 g, 8.75 mmol), 2-amino-2-(4-bromophenyl)acetamide (D1) (2.0 g) and H-Y Zeolites (2.0 g) in methanol (50 ml) was refluxed under argon overnight. The mixture was filtered through Kieselguhr and the solvent was removed to give the title compound as a yellow solid (2.13 g).

Mass Spectrum (LC/MS): Found 295/7 (MH⁺). Ret. time 1.55 min.

¹H NMR (D₆-DMSO) δ: 1.6-1.85 (6H, m), 3.33 (1H, m), 3.63 (1H, m), 4.52 (1H, m), 7.40 (2H, m), 7.53 (2H, m), 8.54 (1H, br).

### Description 3. 3-(4-Bromophenyl)-1,4-diazaspiro[4.4]non-3-en-2-one

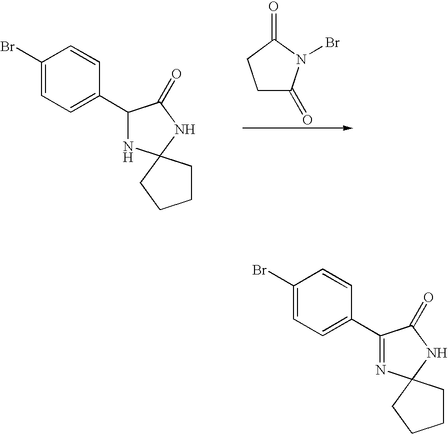

3-(4-Bromophenyl)-1,4-diazaspiro[4,4]nonan-2-one (D2) (2.13 g) in DCM (100 ml) was treated with N-bromosuccinimide (1.27 g) then stirred under argon overnight. Sodium bicarbonate solution was added and the mixture was stirred for 1 hour, then separated. The DCM layer was washed with brine, dried (sodium sulphate) and the solvent was removed to give the title compound as a fawn solid (2.08 g).

Mass Spectrum (LC/MS): Found 293/5 (MH⁺). Ret. time 1.5 min.

¹H NMR (D₆-DMSO) δ:1.85-2.0 (4H, m), 2.1-2.25 (4H, m), 7.40 (1H, br), 7.58 (2H, m), 8.31 (2H, m).

### Description 4. 3-[4-(4-Methyl-1H-imidazol-1-yl)phenyl]-1,4-diazaspiro[4.4]non-3-en-2-one and 3-[4-(5-methyl-1H-imidazol-1-yl)phenyl]-1,4-diazaspiro[4.4]non-3-en-2-one

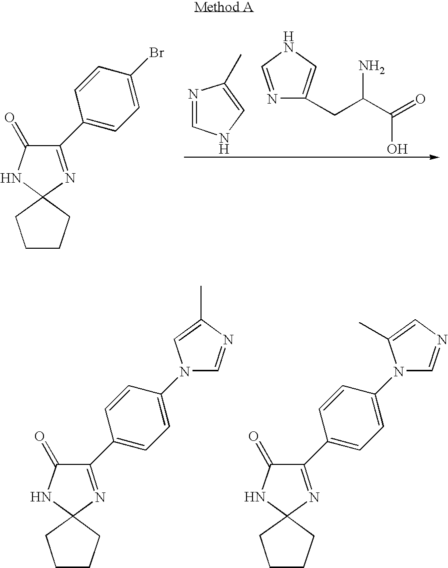

Copper (I) iodide (19.49 mg) and D-histidine (31.8 mg) in DMSO (15 ml) was heated under argon at 110° C. for 40 minutes when 3-(4-bromophenyl)-1,4-diazaspiro[4.4]non-3-en-2-one (D3) (300 mg), 4-methylimidazole (101 mg) and potassium carbonate (283 mg) was added and heating was continued at 110° C. for 3 days. The reaction mixture was poured into a mixture of sodium bicarbonate solution and ethyl acetate and then stirred for 1 hour, filtered and the filtrate was separated. The ethyl acetate layer was dried over sodium sulphate, evaporated and the residue was chromatographed on a silica column eluted with 0-5% 2M methanolic ammonia/DCM to give the title compounds as a white solid (0.27 g). NMR indicated approx. 5:1 mixture of 4-methyl: 5-methyl isomers.

Mass Spectrum (LC/MS): Found 295 (MH⁺). Ret. time 1.1 min.

¹H NMR (CDCl₃) δ: 1.95 (4H, m), 2.10 (4H, m), 2.15 (0.6H, s), 2.31 (2.4H, m), 6.93 (0.2H, s), 7.08 (0.8H, s), 7.35 (2.2H, m), 7.62 (0.2H, s), 7.85 (1.6H, m), 8.53 (2H, m).

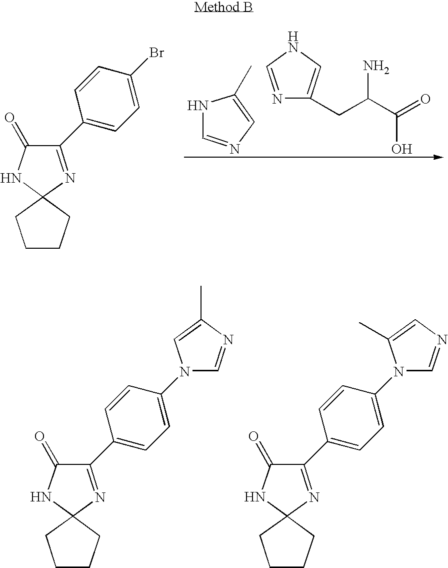

Copper (1) iodide (7.8 g) and L-histidine (12.70 g) in DMSO (300 ml) was heated under argon for 50 minutes at 120° C. when 4-methylimidazole (33.6 g), 3-(4-bromophenyl)-1,4-diazaspiro[4.4]non-3-en-2-one (D3) (20 g) and potassium carbonate (18.86 g) was added and heating was continued for 2.5 hours at 120° C.; and then a further 14 hours. The mixture was poured into a mixture of saturated sodium bicarbonate solution (˜600 ml), water (1000 ml) and ethyl acetate (1500 ml). The mixture was stirred for 30 minutes, kieselguhr ˜50 g was added and the mixture was filtered through kieselguhr and the filtrate was separated (these procedures were done keeping the mixture warm ˜35° C.). The aqueous was then extracted with warm ethyl acetate (3×500 ml) (˜35° C.). The solid from the filtration was washed with boiling ethyl acetate (˜2500 ml). The combined ethyl acetate fractions were washed with brine at ˜(˜35° C). and dried over sodium sulphate at ˜(˜35° C). , evaporated to ˜350 ml and the precipitate was collected by filtration to give a pale green/blue solid, which was dried overnight, and was treated with methanol (150 ml) and 5M HCl (15 ml) to give a yellow solution. This was loaded onto a 70 g SCX cartridge which had been treated with methanol. The cartridge was washed with methanol then eluted with 2M methanolic ammonia. The ammonia fractions were evaporated and the residue was treated with a mixture of ethyl acetate and ether. The white product was collected by filtration to give the title compound (7.64 g). Sample contains <5% of the 5-isomer as estimated by NMR.

Mass Spectrum (LC/MS): Found 295 (MH⁺). Ret. time 1.08 min.

¹H NMR (CDCl3) δ: 1.95 (4H, m), 2.10 (4H, m), 2.31 (3H, m), 6.93 (did not integrate, s), 7.08 (1H, s), 7.45 (2H, m), 7.79 (1H, m),), 7.85 (1H, m), 8.53 (2H, m).

### Description 5. 3-(4-Bromophenyl)-1,4-diazaspiro[4.5]decan-2-one

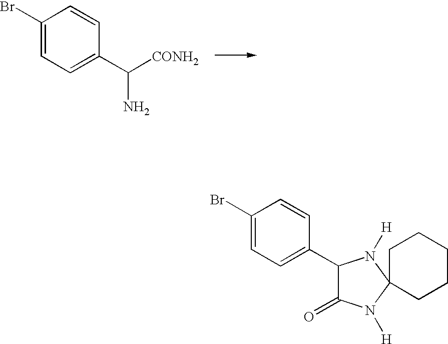

A mixture of 2-amino-2-(4-bromophenyl)acetamide (2.69 g; D1), cyclohexanone (1.22 ml; leg) and H-Y Zeolites (2.69 g) in methanol (100 ml) was stirred vigorously at 80° C. for 16 h under argon. After cooling, the mixture was filtered through celite, washing well with methanol. The filtrate was evaporated at reduced pressure to give the title compound as a white solid (2.22 g).

¹H NMR (D₆-DMSO) δ: 1.30-1.37 (2H, m), 1.50-1.62 (8H, m), 3.50 (1H, d), 4.56 (1H, d), 7.43 (2H, d), 7.51 (2H, d), 8.63 (1H,s). Mass Spectrum (Electrospray LC/MS): Found 309 & 311 (MH⁺). C₁₄H₁₇⁷⁹BrN₂O requires 308 and C₁₄H₁₇⁸¹BrN₂O requires 310. Ret. time 2.21 min.

### Description 6. 3-(4-Bromophenyl)-1,4-diazaspiro[4.5]dec-3-en-2-one

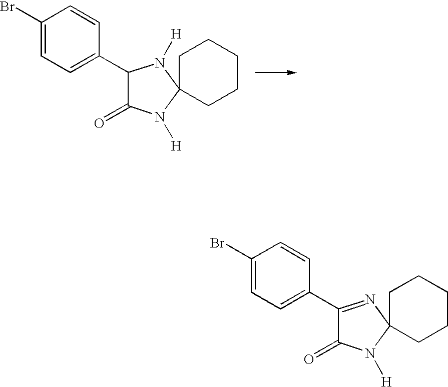

3-(4-Bromophenyl)-1,4-diazaspiro[4.5]decan-2-one (2.22 g; D5) was dissolved in DCM (50 ml) and stirred at room temperature for 16 hours under an atmosphere of argon with N-bromosuccinimide (1.29 g; 1 eq) A solution of saturated sodium hydrogen carbonate (100 ml) was then added and stirring continued for 1 hour at room temperature. The organic layer was separated, dried (MgSO₄) and evaporated at reduced pressure. The residue was triturated with hexane to yield the title compound (2.18 g) as a yellow solid.

¹H NMR (D₆-DMSO) δ: 1.42-1.88 (10H, m), 7.71 (2H, d), 8.28 (2H, d), 10.30 (1H,br s).

Mass Spectrum (Electrospray LC/MS): Found 307 & 309 (MH⁺). C₁₄H₁₅⁷⁹BrN₂O requires 306 and C₁₄H₁₅⁸¹BrN₂O requires 308. Ret. time 3.08 min.

### Description 7. 3-[4-(5-methyl-1H-imidazol-1-yl)phenyl]-1,4-diazaspiro[4.5]dec-3-en-2-one and 3-[4-(4-methyl-1H-imidazol-1-yl)phenyl]-1,4-diazaspiro[4.5]dec-3-en-2-one

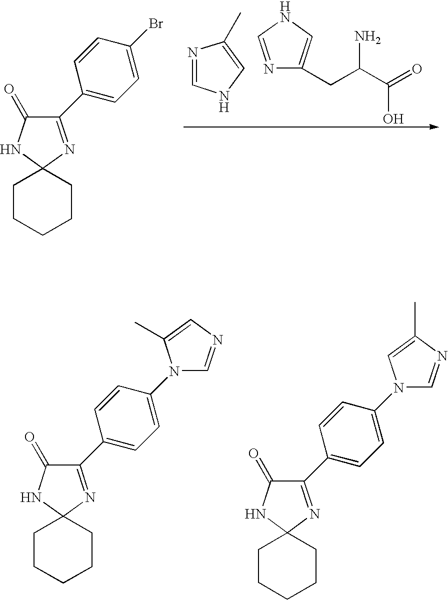

Copper (I) iodide (0.056 g; 0.293 mmol) and D-histidine (0.091 g; 0.586 mmol) in DMSO (15 ml) was heated under argon at 110° C. for 40 minutes when 3-(4-bromophenyl)-1,4-diazaspiro[4.5]dec-3-en-2-one (D6; 0.9 g), 4-methylimidazole (0.241 g; 2.93 mmol) and potassium carbonate (0.810 g; 5.86 mmol) was added and heating was continued at 110° C. for 5 days when the mixture was poured into a mixture of sodium bicarbonate solution and ethyl acetate. The mixture was stirred for 1 hour, filtered and the filtrate was separated. The ethyl acetate layer was dried over sodium sulphate, evaporated and the residue was chromatographed on a silica column eluted with 0-10% 2M methanolic ammonia/DCM to give the title compounds (0.441 g) as a white solid. NMR indicates approx. 4:1 mixture of 4-methyl: 5-methyl isomers.

Mass Spectrum (LC/MS): Found 295 (MH⁺). Ret. time 1.1 min.

¹H NMR (CDCl₃) δ: 1.5-1.7 (obs, m), 1.9-2.10 (4H, m), 2.21 (0.6H, s), 2.31 (2.4H, m), 6.93 (0.2H, s), 7.08 (0.8H, s), 7.35 (2.2H, m), 7.60 (0.2H, s), 7.85 (1.6H, m) 8.55 (2H, m).

### Description 8. 2-Bromo-N[3-(trifluoromethyl)phenyl]acetamide

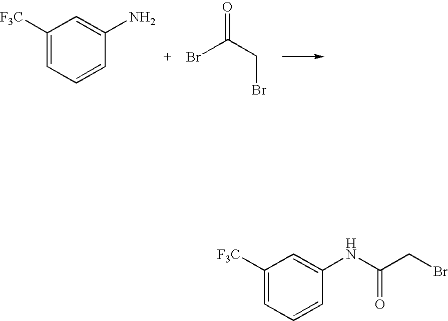

A stirred solution of 3-(trifluoromethyl)aniline (2.0 g, 0.012 mol) in DCM (60 ml) at 10° C. under argon was treated dropwise over 5 minutes with bromoacetyl bromide (1.2 ml, 0.0137 mol). A white precipitate formed. This was allowed to warm to room temperature with good stirring over 1.5 hours, then treated with solid sodium hydrogen carbonate (1.65 g, 0.0196 mol) and stirred well for 40 minutes. The mixture was treated with water (100 ml), stirred well for 10 minutes, then the dichloromethane layer was isolated by passage through a phase separation cartridge and concentrated under vacuum to afford the title compound as a colourless oil (3.65 g). Mass Spectrum (Electrospray LC/MS): Found 282 (MH⁺). C₉H₇⁷⁹BrF₃NO requires 281. Ret. time 2.74 min.

¹H NMR δ (CDCl₃; 400 MHz): 4.05 (2H, s), 7.40-7.53 (2H, m), 7.76 (1H, d), 7.83 (1H, s), 8.24 (1H, br s).

### Description 9. 3-[4-(1H-Imidazol-1-yl)phenyl]-1,4-diazaspiro[4.5]dec-3-en-2-one

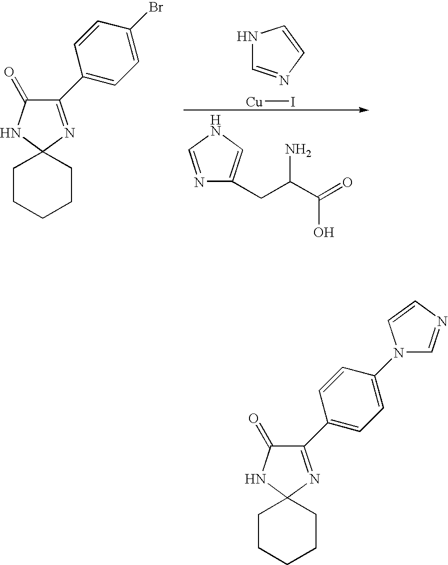

Copper (I) iodide (19.05 mg) and D-histidine (31.0 mg) in DMSO (5 ml) was heated at 100° C. under an atmosphere of argon for 40 minutes when imidazole (82 mg), 3-(4-bromophenyl)-1,4-diazaspiro[4.5]dec-3-en-2-one (D6) (307 mg) and potassium carbonate (276 mg) was added and heating was continued for 2 days. The mixture was poured into a mixture of sodium bicarbonate solution and ethyl acetate which was stirred for 30 minutes, then the organic layer was separated, washed with brine and dried over sodium sulphate. Evaporation gave a fawn solid which was dissolved in a mixture of methanol and DCM, treated with silica and evaporated. The solid was loaded onto a silica chromatography column which was eluted with a gradient of 0-50% MeOH—NH₃-DCM to give the title compound as a white solid (133.0 mg).

¹H NMR (CDCl₃) δ: 1.5-1.8 (obs, m), 2.0 (4H, m), 7.25 (obs, m), 7.36 (1H, m), 7.48 (2H, m), 7.83 (1H, m), 7.95 (1H, m), 8.57 (2H, m).

Mass Spectrum (LC/MS): Found 295 (MH⁺). Ret. time 1.57 min.

### Description 10. 3-[4-(2-Methyl-1H-imidazol-1-yl)phenyl]-1,4-diazaspiro[4.5]dec-3-en-2-one

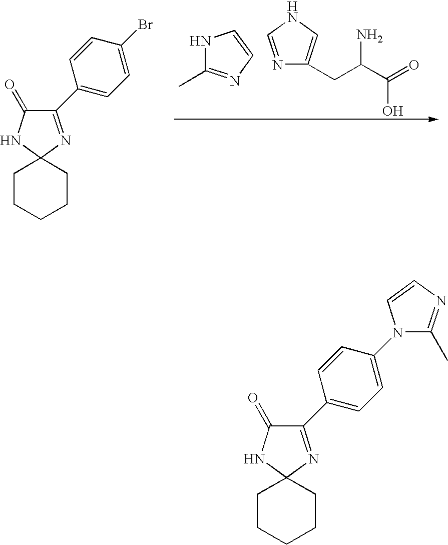

Copper (I) iodide (0.038 g) and D-histidine (0.062 g), in DMSO (10 ml) was heated at 115° C. for 30 minutes under a atmosphere of argon. 3-(4-Bromophenyl)-1,4-diazaspiro[4.5]dec-3-en-2-one (D6) (0.614 g) and potassium carbonate (0.553 g) was added and heating was continued for 5 days when the mixture was poured into a mixture of sodium bicarbonate solution and ethyl acetate. The mixture was stirred for 2 hours, filtered and the filtrate was separated. The ethyl acetate layer was dried over sodium sulphate, evaporated and the residue was chromatographed on a silica column eluted with 0-5% 2M methanolic to give the title compound (0.252 g).

¹H NMR (CDCl₃) δ: 1.5-1.8 (obs, t), 1.9-2.1 (4H, m), 2.42 (3H, s) 7.05 (2H, m), 7.38 (2H, m), 7.70 (1H, br), 8.61 (2H, m).

Mass Spectrum (LC/MS): Found 308 (MH⁺). Ret. time 1.59 min.

### Description 11. 3-[4-(2-Propyl-1H-imidazol-1-yl)phenyl]-1,4-diazaspiro[4.5]dec-3-en-2-one

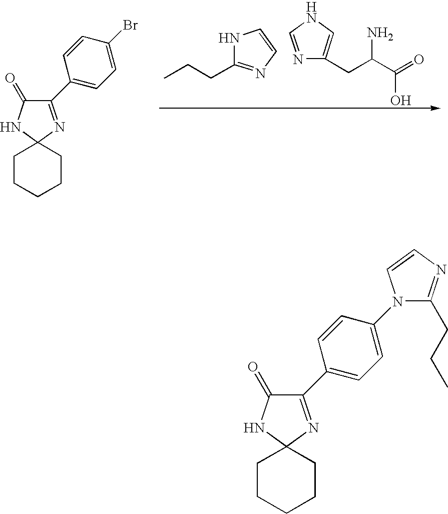

Copper (I) Iodide (0.038 g) and D-histidine (0.062 g) in DMSO (15 ml) was stirred under an atmosphere of argon at 110° C. for 30 minutes when 3-(4-bromophenyl)-1,4-diazaspiro[4.5]dec-3-en-2-one (D6) (0.614 g), 2-propylimidazole (264 mg) and potassium carbonate (0.553 g) was added and heating was continued at 110° C. for 5 days when the mixture was poured into a mixture of sodium bicarbonate solution and ethyl acetate. The mixture was stirred for 2 hours, filtered and the filtrate was separated. The ethyl acetate layer was dried over sodium sulphate, evaporated and the residue was chromatographed on a silica column eluted with 0-5% 2M methanolic ammonia/DCM to give the title compound (0.2 g).

¹H NMR (CDCl₃) δ: 1.4 (3H, t), 1.5-1.85 (obs, m), 1.9-2.1 (4H, m) 2.7 (2H, t), 7.00 (1H, m), 7.09 (1H, m), 7.4 (2H, m), 7.82 (1H, br), 8.55 (2H, m).

Mass Spectrum (LC/MS): Found 337 (MH⁺). Ret. time 1.87 min.

### Description 12. 2-Bromo-N-(3,5-difluorophenyl)acetamide

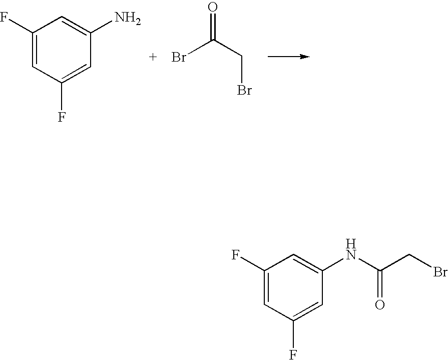

A mixture of 3,5-difluoroaniline (10 g; 77.45 mmol) and bromoacetyl bromide (6.73 ml; 77.45 mmol) in anhydrous dioxan (100 ml) was refluxed for 1.5 h, cooled to room temperature and diluted with water (400 ml) to afford a colourless gum. The mother liquors were decanted and water (200 ml) added, followed by ethyl acetate (300 ml). After stirring for 10 min the layers were separated and the organics dried (Na₂SO₄) and evaporated under reduced pressure. Recrystallisation from ethyl acetate-pentane afforded the title product as pale yellow crystals (6.5 g). ¹H NMR (CDCl₃) δ: 4.02 (2H, s), 6.60-6.65 (1H, m), 7.14-7.20 (2H, m), and 8.16 (1H, br s).

### Description 13. 3-(4-Bromophenyl)-7-oxa-1,4-diazaspiro[4.4]nonan-2-one

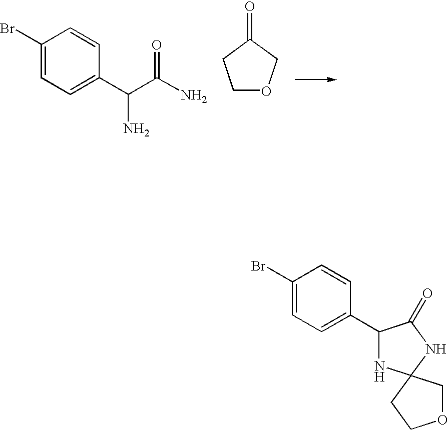

A mixture of dihydro-3(2H)-furanone (0.951 g) and 2-amino-2-(4-bromophenyl)acetamide (D1) (2.3 g) and H-Y Zeolites (2.3 g) in methanol (125 ml) was refluxed under argon overnight. The mixture was filtered through Kieselguhr and the solvent was removed to give the title compound as an off white solid foam (2.59 g), which was used without further purification.

Mass spectrum Found 297/9 (MH⁺).

### Description 14. 3-(4-Bromophenyl)-7-oxa-1,4-diazaspiro[4.4]non-3-en-2-one

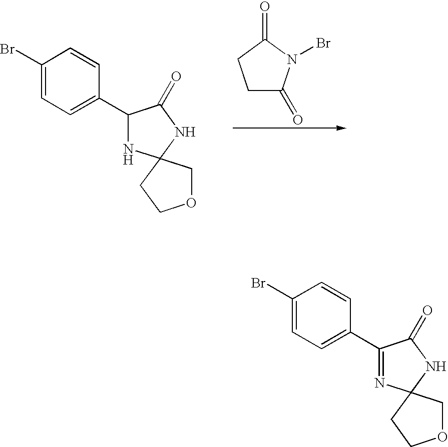

3-(4-Bromophenyl)-7-oxa-1,4-diazaspiro[4.4]nonan-2-one (D13) (2.52 g) in DCM (50 ml) was treated with N-bromosuccinimide (1.64 g) then stirred under argon overnight. Sodium bicarbonate solution was added and the mixture was stirred for 1 hour then separated. The DCM layer was washed with brine, dried sodium sulphate and the solvent was removed to give a fawn solid, this was chromatographed, on a silica column eluted with 0-5% MeOH-DCM to give the title compound as a brown solid (1.22 g).

¹H NMR (CDCl₃) δ: 2.26 (1H, m), 2.60 (1H, m), 3.85 (1H, m), 4.06 (1H, m), 4.25 (2H, m), 7.62 (2H, m), 7.86 (1H, br), 8.32 (2H, m).

Mass spectrum Found 295/7 (MH⁺).

### Description 15. 3-(4-Bromophenyl)-7-oxa-1,4-diazaspiro[4.5]decan-2-one

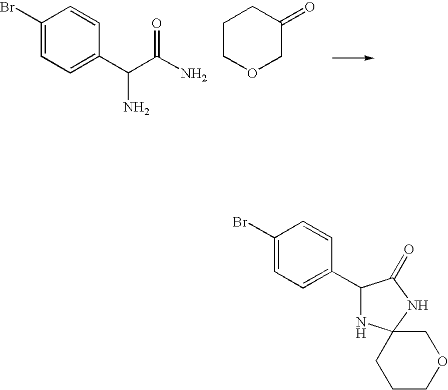

A mixture of tetrahydropyran-3-one (0.25 g) and 2-amino-2-(4-bromophenyl)acetamide (D1) (0.572 g) and H-Y Zeolites (0.57 g) in methanol (25 ml) was refluxed under argon overnight. The mixture was filtered through Kieselguhr and the solvent was removed to give crude title compound as a yellow solid which was used without purification (712 mg). Mass spectrum Found 311/3 (MH⁺).

### Description 16. 3-(4-Bromophenyl)-7-oxa-1,4-diazaspiro[4.5]dec-3-en-2-one

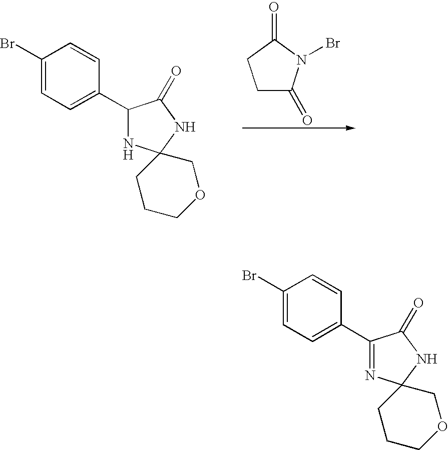

3-(4-Bromophenyl)-7-oxa-1,4-diazaspiro[4.5]decan-2-one (D15) (0.771 g) in DCM (50 ml) was treated with N-bromosuccinimide (0.463 g) then stirred under argon overnight. Sodium bicarbonate solution was added and the mixture was stirred for 1 hour then separated. The DCM layer was washed with brine and dried over sodium sulphate and the solvent was removed to give a fawn solid. This was triturated with DCM and the white product was collected by filtration to give the title compound. (209 mg).

¹H NMR (D₆-DMSO) δ: 1.65 (1H, m), 1.85 (2H, m), 2.08 (1H, m), 3.45 (1H, m), 3.6-3.75 (2H, m), 3.8 (1H, m), 7.72 (2H, m), 8.28 (2H, m), 10.45 (1H, s).

### Description 17. 3-[4-(2-Pyridinyl)phenyl]-7-oxa-1,4-diazaspiro[4.4]non-3-en-2-one

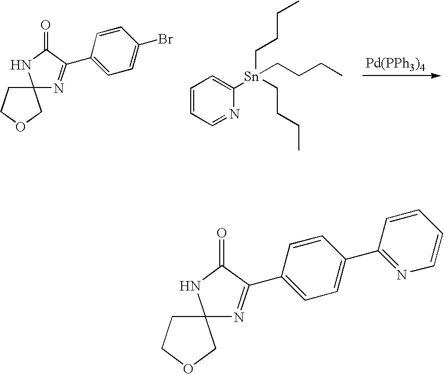

A degassed solution of 3-(4-bromophenyl)-7-oxa-1,4-diazaspiro[4.4]non-3-en-2-one (D14) (401 mg), 2-(tributylstannanyl)pyridine (601 mg, 1.632 mmol) and tetrakis(triphenylphosphine)palladium(0) (79 mg, 0.068 mmol) in toluene (15 ml) was heated at reflux, under argon for 10 hours. The reaction mixture was cooled, diluted with MeOH (10 ml) and passed through an SCX column (10 g), washing through with MeOH (50 ml) then eluting with 2M NH₃-MeOH (20 ml). Evaporation of the NH₃-MeOH and trituration of the resulting gum with diethyl ether afforded the title compound as a yellow powder (110 mg).

¹H NMR (D₆-DMSO) δ: 2.22 (1H, m), 2.34 (1H, m), 3.75 (1H, m), 3.86 (1H, m) 4.10 (2H, m), 7.42 (1H, m), 7.93 (1H, m), 8.07 (1H, m), 8.26 (2H, m), 8.46 (2H, m), 8.71 (1H, m), 10.25 (1H,s).

Mass Spectrum (LC/MS): Found 294 (MH⁺). Ret. time 1.29 min.

### Description 18. 3-[4-(2-Pyridinyl)phenyl]-1,4-diazaspiro[4.4]non-3-en-2-one

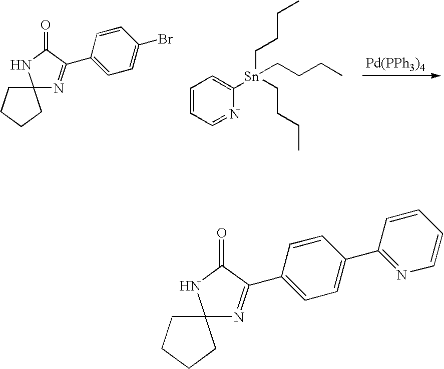

A degassed solution of 3-(4-bromophenyl)-1,4-diazaspiro[4.4]non-3-en-2-one (D3) (400 mg), 2-(tributylstannanyl)pyridine (603 mg, 1.637 mmol) and tetrakis(triphenylphosphine)palladium(0) (79 mg, 0.068 mmol) in toluene (15 ml) was heated at reflux, under argon for 12 hours. The reaction mixture was cooled, diluted with MeOH and passed through an SCX column, washing through with MeOH then eluting the product with 2M NH₃-MeOH. Probably as a result of over-loading of the column significant amounts of product was present in the initial MeOH washings. The MeOH was evaporated and the residue triturated with diethyl ether to afford the title compound as a pale yellow powder (160 mg).

¹H NMR (D₆-DMSO) δ: 1.9 (8H, m), 7.41 (1H, m), 7.93 (1H, m), 8.06 (1H, d), 8.23 (2H, m), 8.44 (2H, m), 8.71 (1H, m), 10.10 (1H, s).

Mass Spectrum (LC/MS): Found 292 (MH⁺). Ret. time 1.68 min.

### Description 19. Amino(3-bromophenyl)acetic acid

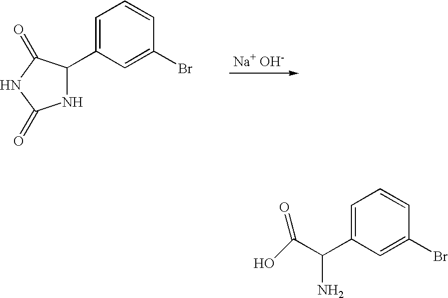

A stirred mixture of 5-(3-bromophenyl)-2,4-imidazolidinedione (prepared using a similar method to that disclosed in Pharmazie 1981, 36, 467) (7.8 g, 30.6 mmol) and sodium hydroxide (10 g, 250 mmol) in water (100 ml) was heated under reflux for 4 days, then allowed to cool to room temperature and stand for 6 days. The stirred reaction mixture was then treated with acetic acid to pH 7 and the resulting precipitate filtered off, washed with water and dried under vacuum to leave a white solid. This material was suspended in water (200 ml), adjusted to pH 2 by addition of conc HCl acid and then stirred vigorously for 2 hours. The remaining solid was filtered off and the filtrate adjusted to pH 7 by addition of conc. NaOH solution producing a precipitate. This mixture was cooled to 7° C. by standing in a fridge for 2 hours, then the solid was filtered off, washed with water and dried under vacuum at 50° C. to afford the title compound as a white solid (5.70 g).

¹H NMR (D₆-DMSO) δ: 4.25 (1H, s), 7.30 (1H, m), 7.40 (1H, d), 7.50 (1H, d), 7.63 (1H, s). OH and NH₂ signals obscured by broad peaks at 3.34 and 8.05.

### Description 20. Methyl amino(3-bromophenyl)acetate

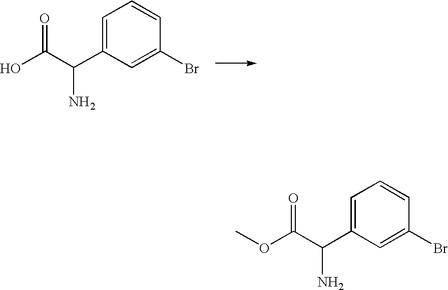

A stirred suspension of amino(3-bromophenyl)acetic acid (D19) (3.0 g) in methanol (50 ml) at room temperature under argon was treated with conc HCl acid (4 ml) and then heated at reflux temperature for 4 hours. The solution was concentrated under vacuum to approx 10 ml volume then added carefully to excess sat. NaHCO₃ solution (100 ml), treated with EtOAc (100 ml) and shaken well. As significant insoluble material was present, the mixture was filtered and the EtOAc layer isolated from the filtrate, dried (Na₂SO₄) and concentrated under vacuum to leave the title compound as a pale yellow oil (2.4 g).

¹H NMR (CDCl₃) δ: 1.90 (2H, br s), 3.71 (3H, s), 4.59 (1H, s), 7.25 (1H, m), 7.31 (1H, d), 7.43 (1H, d), 7.57 (1H, s).

### Description 21. 2-Amino-2-(3-bromophenyl)acetamide

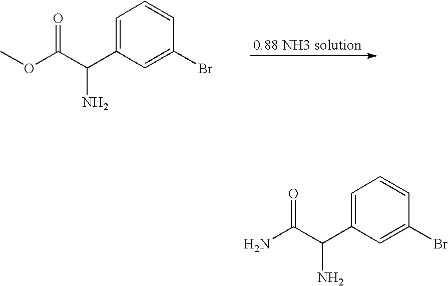

A mixture of methyl amino(3-bromophenyl)acetate (D20) (2.35 g) in 0.88 NH₃ solution (50 ml) at room temperature under argon was stirred well for 18 hours affording a homogeneous solution, which was then extracted with DCM (3×30 ml) and the extract dried (Na₂SO₄) and concentrated under vacuum to leave the title compound as a white solid (1.51 g).

¹H NMR (D₆-DMSO) δ: 2.23 (2H, s), 4.30 (1H, s), 7.08 (1H, s), 7.26 (1H, m), 7.40-7.46 (2H, m), 7.51 (1H, s), 7.60 (1H, s).

### Description 22. 3-(3-Bromophenyl)-1,4-diazaspiro[4.5]decan-2-one

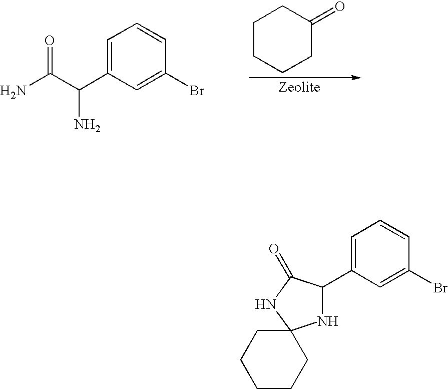

A stirred mixture of 2-amino-2-(3-bromophenyl)acetamide (D21) (1.5 g) in ethanol (60 ml) at room temperature under argon was treated with cyclohexanone (0.713 ml, 6.88 mmol) followed by H-Y Zeolites (2.0 g, 6.55 mmol) and then heated under reflux for 18 hours. The reaction mixture was allowed to cool, filtered through Kieselguhr and the filtrate concentrated under vacuum. The residue was triturated with Et₂O (50 ml) and the solid filtered off and dried under vacuum to afford the title compound as a white solid (1.52 g).

Mass Spectrum (LC/MS): Found 309/311 (MH⁺). Ret. time 1.99 min.

¹H NMR (CDCl₃) δ: 1.35-1.60 (4H, m), 1.60-1.82 (6H, m), 2.22 (1H, d), 4.69 (1H, d), 71.13-7.32 (2H, m), 7.40-7.50 (2H, m), 7.71 (1H, s).

### Description 23. 3-(3-Bromophenyl)-1,4-diazaspiro[4.5]dec-3-en-2-one

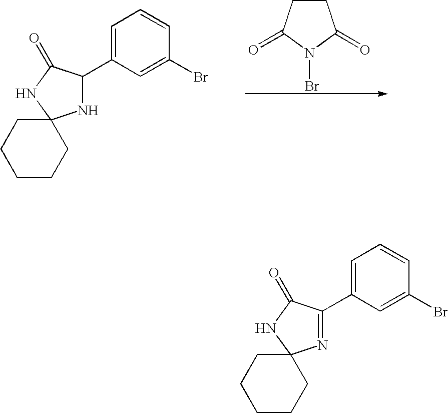

A stirred solution of 3-(3-bromophenyl)-1,4-diazaspiro[4.5]decan-2-one (D22) (1.50 g) in dichloromethane (35 ml) at room temperature under argon was treated with N-bromosuccinimide (0.907 g, 5.09 mmol) and maintained at room temperature for 18 hours producing a yellow heterogeneous mixture. This was diluted with DCM (35 ml), treated with 5% NaHCO₃ solution (60 ml) and stirred well for 2 hours. At this stage the yellow colour had been lost and a precipitate was present. The solid was filtered off, washed with water and DCM and dried under vacuum to afford the title compound as a white solid (1.25 g).

Mass Spectrum (LC/MS): Found 307/309 (MH⁺). Ret. time 2.76 min.

¹H NMR (D₆-DMSO) δ: 1.40-1.85 (10H, m), 7.48 (1H, t), 7.77 (1H, m), 8.31 (1H, m), 8.50 (1H, s), 10.32 (1H, br s).

### Description 24. 3-[3-(2-Pyridinyl)phenyl]-1,4-diazaspiro[4.5]dec-3-en-2-one

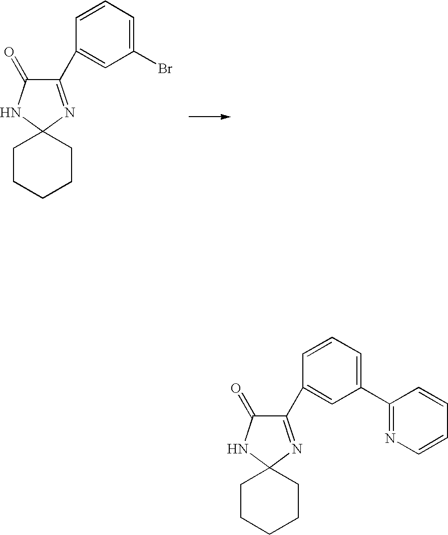

A mixture of 3-(3-bromophenyl)-1,4-diazaspiro[4.5]dec-3-en-2-one (D23) (100 mg), 2-(tributylstannanyl)pyridine (144 mg, 0.391 mmol) and tetrakis(triphenylphosphine)palladium(0) (18.81 mg, 0.016 mmol) in toluene (10 ml) were heated at reflux for 26 hours followed by a further 14 hours at room temperature (approx due to a power failure). A further quantity of tetrakis(triphenylphosphine)palladium(0) (18.81 mg, 0.016 mmol) and 2-(tributylstannanyl)pyridine (144 mg, 0.391 mmol) was added and heating was continued at reflux for 18 hours. The resulting solution was allowed to cool, 5 ml of water was added and the mixture was then evaporated to dryness. The resulting compound was purified using 5 g SCX-2, which was pre conditioned with DCM. The cartridge was washed with DCM (2CV), and MeOH (2CV). The compound was eluted using 0.5M NH₃/MeOH and the eluant was evaporated to dryness under reduced pressure. The residue was purified via Biotage (20-60% ethyl acetate/hexane, 12M column). The desired fractions were combined and evaporated to dryness to give the title compound as a white solid (21 mg). Mass Spectrum (Electrospray LC/MS): Found 306 (MH⁺). C₁₉H₁₉N₃O requires 305. Ret. time 1.90 min.

### Description 25. 2-Bromo-N-(3-chlorophenyl)acetamide

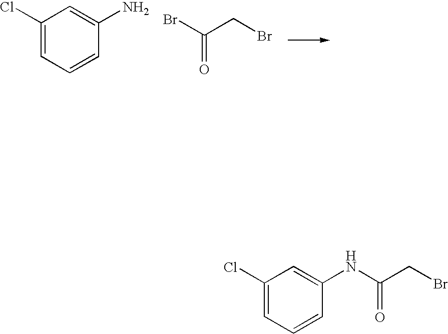

A stirred solution of 3-chloroaniline (2 g, 15.68 mmol) in dichloromethane (60 ml) under argon was cooled to 10° C. before the addition of bromoacetyl bromide (1.515 ml, 17.42 mmol) dropwise over 5 minutes, resulting in the formation of a white precipitate. The resulting solution was allowed to warm to room temperature and stirred for 1.5 hours. Solid sodium hydrogen carbonate was added (2 g). The solution was stirred at room temperature for 20 mins. Water was then added and stirring continued for 60 mins. The layers were separated using a phase separation cartridge and the organic layer was evaporated to dryness to give the title compound as a white solid (3.94 g). Mass Spectrum (Electrospray LC/MS): Found 249 (MH⁺). C₈H₇BrClNO requires 248. Ret. time 2.25 min.

### Description 26. 3-[4-(2-Pyridinyl)phenyl]-7-oxa-1,4-diazaspiro[4.5]dec-3-en-2-one

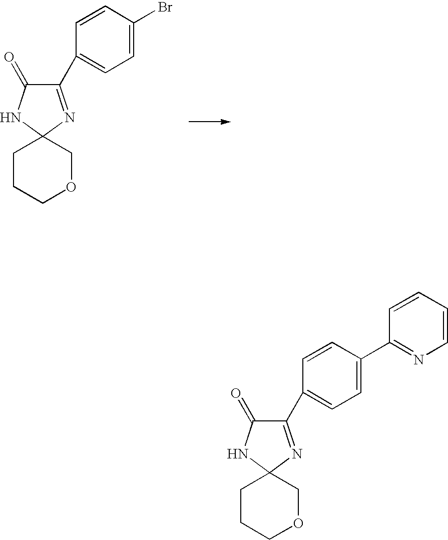

A degassed solution of 3-(4-bromophenyl)-7-oxa-1,4-diazaspiro[4.5]dec-3-en-2-one (D15) (500 mg), 2-(tributylstannanyl)pyridine (714 mg, 1.941 mmol) and tetrakis(triphenylphosphine)palladium(0) (93 mg, 0.081 mmol) in toluene (15 ml) was heated at reflux, under argon for 10 h. The reaction mixture was cooled, diluted with MeOH (10 ml) and passed through an SCX column (10 g), washing through with MeOH (50 ml) then eluting with 2M NH₃-MeOH (20 ml). Evaporation of the NH₃-MeOH and trituration of the resulting gum with diethyl ether afforded the title compound as a beige powder (320 mg, 1.041 mmol).

¹H NMR (CDCl₃) δ: 1.72 (1H, m), 1.85-2.00 (2H, m), 2.33(1H, m), 3.50 (2H, m), 3.94 (1H, d), 4.06 (1H, d), 7.28 (1H, m, obscured by CHCl₃), 7.80 (2H, m), 8.12 (3H, m), 8.55 (2H, m), 8.73 (1H, m).

Mass Spectrum (Electrospray LC/MS): Found 308 (MH⁺). C₁₈H₁₇N₃O₂ requires 307. Ret. time 1.44 min

### Description 27. 3-[4-(2,4-Dimethyl-1H-imidazol-1-yl)phenyl]-1,4-diazaspiro[4.4]non-3-en-2-one

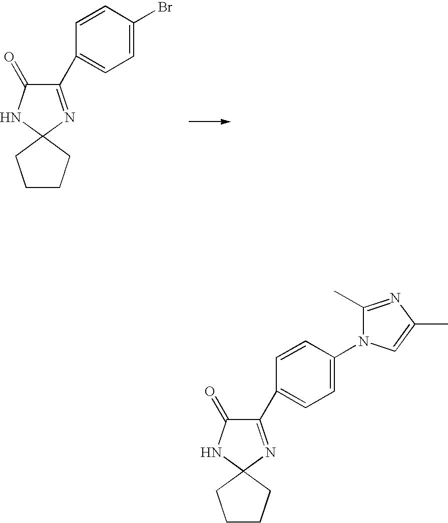

A stirred solution of 3-(4-bromophenyl)-1,4-diazaspiro[4.4]non-3-en-2-one (D3) (770 mg2,4-dimethyl-1H-imidazole (379 mg, 3.94 mmol) and copper bis(2,2,6,6-tetramethyl-3,5-heptanedionate) (337 mg, 0.788 mmol) in DMF (3 ml) at room temperature, under argon was treated with potassium tert-butoxide (1179 mg, 10.51 mmol). After stirring at room temperature for 5 min, the mixture was heated to 120° C. and this temperature was maintained for 16 h. Further batches of the imidazole (379 mg) and copper bis(2,2,6,6-tetramethyl-3,5-heptanedionate) (337 mg) were added and heating was continued for 7 days. The reaction mixture was cooled and diluted with saturated aqueous sodium bicarbonate solution (100 ml) and ethyl acetate (100 ml). After mixing, the mixture was filtered through Kieselghur. The ethyl acetate layer was separated and the aqueous phase re-extracted with ethyl acetate (50 ml). The combined organics were dried (MgSO₄) and evaporated. The residue was applied in MeOH to an SCX column (10 g). After washing with MeOH the product was eluted with 2M NH₃-MeOH. Evaporation afforded a brown gum. Chromatography (10 g isolute column, 0-2% MeOH-DCM) afforded, after solvent removal, material (250 mg) consistent with presence of 2 isomers in approx 9:1 ratio with the major component subsequently confirmed (by ¹H NMR nOe studies at the final product stage) as the title compound.

Mass Spectrum (Electrospray LC/MS): Found 309 (MH⁺). C₁₈H₂₀N₄O requires 308. Ret. time 1.02 min

### Description 28. 3-[4-(4,5-Dimethyl-1H-imidazol-1-yl)phenyl]-1,4-diazaspiro[4.4]non-3-en-2-one

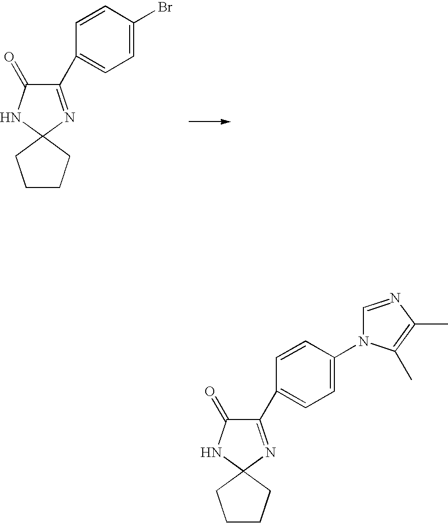

A stirred solution of 3-(4-bromophenyl)-1,4-diazaspiro[4.4]non-3-en-2-one (D3) (500 mg), 4,5-dimethyl imidazole (148 mg, 1.535 mmol) and copper bis(2,2,6,6-tetramethyl-3,5-heptanedionate) (197 mg, 0.461 mmol) in DMF (3 ml) at room temperature, under argon was treated with potassium tert-butoxide (689 mg, 6.14 mmol). After stirring at room temperature for 5 min. the mixture was heated to 120° C. and this temperature was maintained for 36 h. Further batches of the imidazole (148 mg) and copper bis(2,2,6,6-tetramethyl-3,5-heptanedionate) (197 mg) were added and heating was continued for 2 days. Further imidazole (74 mg) and copper bis(2,2,6,6-tetramethyl-3,5-heptanedionate) (98 mg) were added and heating was continued for a further 2 days. The reaction mixture was cooled and diluted with saturated aqueous sodium bicarbonate solution (100 ml) and ethyl acetate (100 ml). After mixing, the mixture was filtered through Kieselghur. The ethyl acetate layer was separated and the aqueous phase re-extracted with ethyl acetate (50 ml). The combined organics were dried (MgSO₄) and evaporated. The residue was applied in MeOH to an SCX column (10 g). After washing with MeOH the product was eluted with 2M NH₃-MeOH. Evaporation afforded a brown gum. Chromatography (10 g isolute column, 0-2% MeOH-DCM) afforded the title compound as a beige foamed solid (156 mg, 0.506 mmol, 33.0% yield).

¹H NMR (CDCl₃) δ: 1.98 (4H, m), 2.11 (7H, m), 2.24(3H, s), 7.36 (2H, m), 7.55 (1H, s), 8.08 (1H, s), 8.53 (2H, m).

Mass Spectrum (Electrospray LC/MS): Found 309 (MH⁺). C₁₈H₂₀N₄O requires 308. Ret. time 1.11 min

### Description 29. 3-[3-(4-Methyl-1H-imidazol-1-yl)phenyl]-1,4-diazaspiro[4.5]dec-3-en-2-one and 3-[3-(5-methyl-1H-imidazol-1-yl)phenyl]-1,4-diazaspiro[4.5]dec-3-en-2-one (approx. 4:1 mixture)

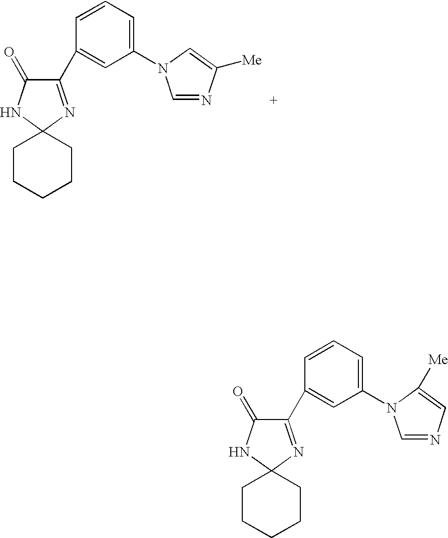

A stirred mixture of 3-(3-bromophenyl)-1,4-diazaspiro[4.5]dec-3-en-2-one (D23) (150 mg, 0.488 mmol), 4-methylimidazole (60.1 mg, 0.732 mmol) and copper bis(2,2,6,6-tetramethyl-3,5-heptanedionate (63.0 mg, 0.146 mmol) in N,N-dimethylformamide (3 ml) at room temperature under argon was treated with potassium t-butoxide (219 mg, 1.953 mmol) and maintained for 5 minutes. The initial dark blue coloured mixture gave way to a pale yellow colour. The reaction mixture was then heated to 120° C. producing a heterogenous dark brown mixture inside 20 minutes. Heating was continued at 120° C. for 44 hours when further 4-methylimidazole (60.1 mg, 0.732 mmol) and copper bis(2,2,6,6-tetramethyl-3,5-heptanedionate (63.0 mg, 0.146 mmol) were added and heating continued at 120° C. for additional 4 days. The reaction mixture was allowed to cool and then treated with 10% Na₂CO₃ solution (25 ml) and EtOAc (40 ml). The mixture was shaken well and then filtered through Kieselguhr. The EtOAc solution was isolated and the aqueous extracted with EtOAc (25 ml). The two EtOAc solutions were combined, dried (Na₂SO₄) and concentrated under vacuum. The residue was dissolved in MeOH (2 ml) and loaded on to a SCX cartridge (2 g) and washed with MeOH (10 ml) and then eluted with 2M NH₃/MeOH (10 ml). The NH₃ solution was concentrated under vacuum to leave a brown oil (178 mg), which was purified by Biotage (12+M column) eluting with 0-10% MeOH/DCM to afford an orange oil (70 mg). NMR suggested this was approx. 4:1 mixture of 4-methyl:5-methyl imidazole isomers (approx. 80% of material) plus some impurities (approx. 20% of the material). This material was used directly in the next step. Mass Spectrum (Electrospray LC/MS): Found 309 (MH⁺). C₁₈H₂₀N₄O requires 308. Ret. time 1.19 min.

### Description 30. 3-[4-(2-Methyl-1H-imidazol-1-yl)phenyl]-1,4-diazaspiro[4.4]non-3-en-2-one

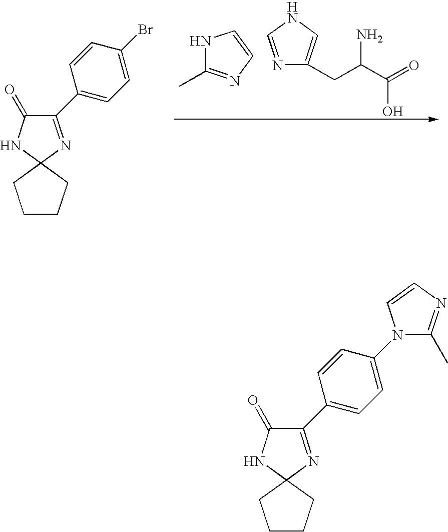

Copper (I) iodide (78 mg) and D-histidine (127 mg) in DMSO (5 ml) was heated under argon for 40 minutes at 115° C. when 3-(4-bromophenyl)-1,4-diazaspiro[4.4]non-3-en-2-one (D3) (0.6 g), 2-methylimidazole (336 mg) and potassium carbonate (0.556 g) were added and heating was continued at 115° C. overnight. The mixture was poured into a mixture of sodium bicarbonate solution and ethyl acetate and stirred for 2 hours, then filtered and the filtrate was separated. The ethyl acetate layer was dried over sodium sulphate, evaporated and the residue was chromatographed on a silica column eluted with 0-5% 2M methanolic ammonia/DCM. The second peak to elute was collected to give the title compound as a white solid (62 mg). Mass Spectrum (LC/MS): Found 295 (MH⁺). Ret. time 0.94 min.

¹H NMR (CDCl₃) δ: 1.95 (4H, m), 2.15 (4H, m), 2.41 (3H, s), 7.05 (2H, m), 7.39 (2H, m), 7.6 (1H, s), 8.54 (2H, m).

### Description 31. chiral 3-[4-(1H-Imidazol-1-yl)phenyl]-7-oxa-1,4-diazaspiro[4.5]dec-3-en-2-one

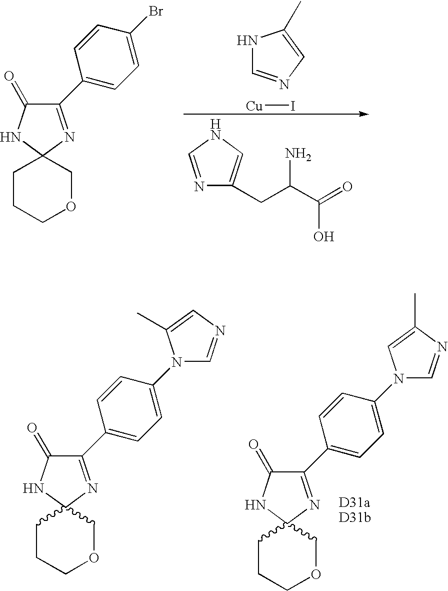

Copper (I) iodide (74 mg) and D-histidine (0.12 g) in DMSO (10 ml) was heated under argon for 60 minutes at 110° C. when 3-(4-bromophenyl)-7-oxa-1,4-diazaspiro[4.5]dec-3-en-2-one (D15) (0.6 g) and 4-methylimidazole (0.319 g) and potassium carbonate (0.322 g) was added and heating was continued overnight at 110° C. Heating was continued for 1.5 extra days. The mixture was poured into a mixture of sodium bicarbonate solution and ethyl acetate. The mixture was stirred for 1 hour, filtered and the filtrate was separated. The ethyl acetate layer was dried over sodium sulphate, evaporated and the residue was chromatographed using eluant of 0-10% 2M NH₃-MeOH-DCM. The main peak was collected and then the isomers were separated by chromatography on a Chiracel OD column eluting with a heptane-ethanol gradient. The first two eluting peaks were not fully resolved so were combined and further purified using Chiracel OJ eluting with a heptane-ethanol gradient to give separately, the two enantiomers of 3-[4-(1H-imidazol-1-yl)phenyl]-7-oxa-1,4-diazaspiro[4.5]dec-3-en-2-one.

Isomer 1

### chiral-3-[4-(1H-imidazol -1-yl)phenyl]-7-oxa-1,4-diazaspiro[4.5]dec-3-en-2-one

Obtained as a clear glass (97 mg).

¹H NMR (CDCl₃) δ: 1.7 (1H, m), 1.86 (1H, m), 1.98 (1H, m), 2.31 (4H, m), 3.5 (1H, m), 3.6 (1H, m), 3.9 (1H, m), 4.08 (1H, m), 7.08 (1H, s), 7.45 (2H, m), 7.79 (1H, m), 7.86 (1H, m), 8.55. (2H, m).

Mass Spectrum (LC/MS): Found 311 (MH⁺). Ret. time 0.93 min.

Isomer 2

### chiral-3-[4-(1H-imidazol -1-yl)phenyl]-7-oxa-1,4-diazaspiro[4.5]dec-3-en-2-one

Obtained as a clear glass (94 mg).

¹H NMR (CDCl₃) δ: 1.74 (1H, m), 1.86 (1H, m), 1.95 (1H, m), 2.3 (4H, m), 3.5 (1H, m), 3.6 (1H, m), 3.9 (1H, m), 4.08 (1H, m), 7.08 (1H, s), 7.45 (2H, m), 7.77 (1H, m), 7.86 (1H, m), 8.6 (2H, m).

Mass Spectrum (LC/MS): Found 311 (MH⁺). Ret. time 0.91 min.

### Description 32. 3-[4-(1-Methyl-1H-imidazol-2-yl)phenyl]-1,4-diazaspiro[4.4]non-3-en-2-one

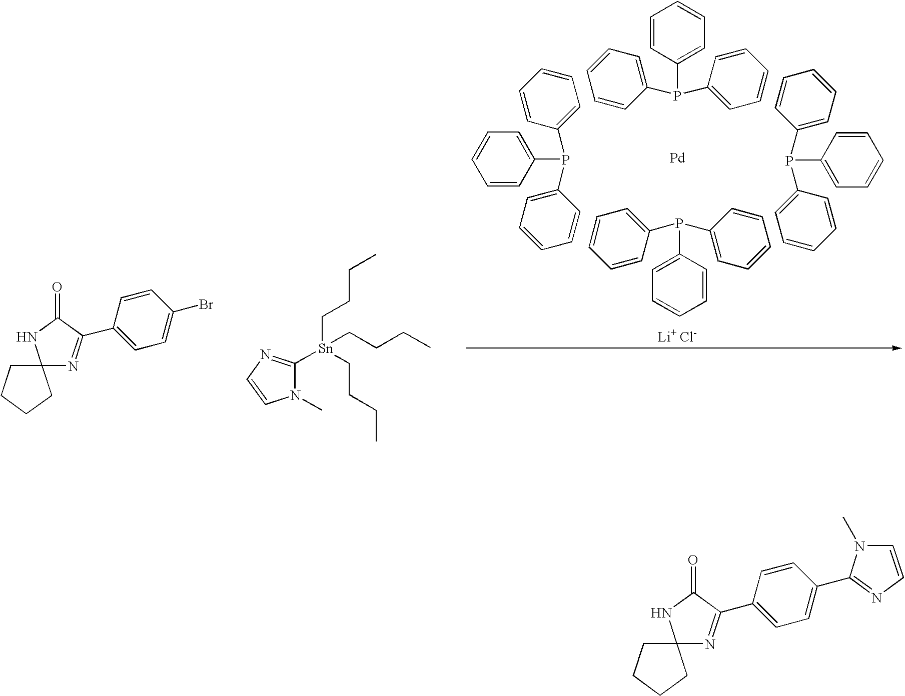

A degassed solution of 3-(4-bromophenyl)-1,4-diazaspiro[4.4]non-3-en-2-one (D3) (500 mg), 1-methyl-2-(tributylstannanyl)-1H-imidazole (760 mg, 2.047 mmol) and tetrakis(triphenylphosphine)palladium(0) (99 mg, 0.085 mmol) in toluene (20 ml) was heated at reflux, under argon for 16 h. A further portion of catalyst (100 mg) was added and heating continued for a further 16 h. Lithium chloride (72.3 mg) was added along with a further 100 mg of tetrakis(triphenylphosphine)palladium(0) and heating was continued for a further 16 h. The reaction mixture was cooled, diluted with MeOH (20 ml) and passed through an SCX column (10 g), washing through with MeOH (50 ml) then eluting with 2M NH₃-MeOH (20 ml). Evaporation of the NH₃-MeOH and trituration of the resulting gum with diethyl ether afforded a yellow gum. Trituration with diethyl ether and MDC afforded a solid (70 mg), which contained a trace of product and mother liquors which evaporated down to give a yellow gum (100 mg) which by LCMS was majority product. MDAP purification afforded the title compound as a white solid (18 mg).

¹H NMR (CDCl₃) δ: 1.94 (4H, m), 2.13 (4H, m), 3.79 (3H, s), 7.01 (1H, s), 7.16 (1H, s), 7.77 (2H, m), 8.48 (2H, m).

Mass Spectrum (LC/MS): Found 295 (MH⁺). C₁₇H₁₈N₄O requires 294. Ret. time 0.94 min.

### EXAMPLE 1a

### 2-{3-[4-(4-Methyl-1H-imidazol-1-yl)phenyl]-2-oxo-1,4-diazaspiro[4.4]non-3-en-1-yl}-N-[3-(trifluoromethyl)phenyl]acetamide hydrochloride (Method A), and

### EXAMPLE 2

### 2-{3-[4-(5-Methyl-1H-imidazol-1-yl)phenyl]-2-oxo-1,4-diazaspiro[4.4]non-3-en-1-yl}-N[3-(trifluoromethyl)phenyl]acetamide

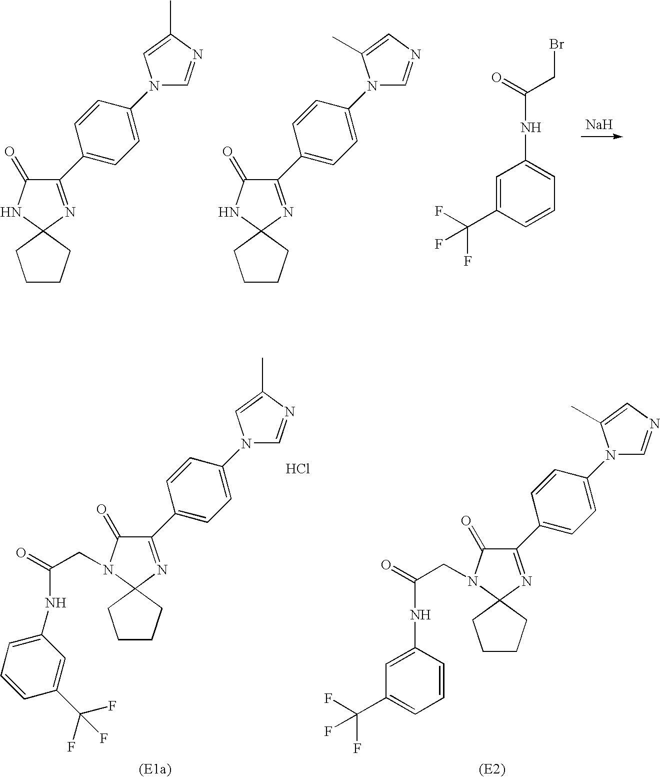

A mixture of 3-[4-(4-methyl-1H-imidazol-1-yl)phenyl]-1,4-diazaspiro[4.4]non-3-en-2-one (120 mg) contaminated with 3-[4-(5-methyl-1H-imidazol-1-yl)phenyl]-1,4-diazaspiro[4.4]non-3-en-2-one (approx. 4:1) ((D4), Method A) in DMF (4 ml) was cooled to ice bath temperature and treated with sodium hydride (60% in oil) (19.57 mg) under an atmosphere of argon. The mixture was stirred for 30 minutes when 2-bromo-N-[3-(trifluoromethyl)phenyl]acetamide (D8) (115 mg) in DMF (2 ml) was added over 1 hour by syringe pump. The mixture was then allowed to warm to room temp overnight. The mixture was purified by multiple MDAPs to give two fractions. These were seperately loaded onto SCX and eluted with 2M-NH₃-MeOH.

First eluting isomer was 2-{3-[4-(5-methyl-1H-imidazol-1-yl)phenyl]-2-oxo-1,4-diazaspiro[4.4]non-3-en-1-yl}-N-[3-(trifluoromethyl)phenyl]acetamide (E2) (15 mg).

¹H NMR (CDCl₃) δ: ˜1.6 (obs, m), 1.88 (2H, m), 1.9-2.2 (4H, m), 2.23 (3H, s), 4.28 (2H, s), 6.99 (1H, m), 7.38 (1H, m), 7.42 (2H, m), 7.52 (1H, m), 7.7 (1H, m), 7.82 (1H, s), 8.59 (2H, m) 9.07 (1H, m).

Mass Spectrum (LC/MS): Found 496 (MH⁺). Ret. time 1.72 min.

Second eluting isomer gave 2-{3-[4-(4-methyl-1H-imidazol-1-yl)phenyl]-2-oxo-1,4-diazaspiro[4.4]non-3-en-1-yl}-N-[3-(trifluoromethyl)phenyl]acetamide, which was converted to the HCl salt with DCM/Et₂O/HCl to give a white solid (E1a) (41 mg).

¹H NMR (D₆-DMSO) δ: 1.7 (2H, m), 1.9-2.1 (4H, m), 2.2 (2H, m), 2.37 (3H, s), 3.9 (1H, m), 4.40 (2H, m), 7.45 (1H, m), 7.58 (2H, m), 7.77 (1H, m), 7.95 (2H, m), 8.13 (2H, m) 8.55 (2H, m), 9.73 (1H, s), 10.8 (1H, s).

¹⁹F NMR (DMSO) δ: −60.4

Mass Spectrum (LC/MS): Found 496 (MH⁺). Ret. time 1.86 min.

### EXAMPLE 1b

### 2-{3-[4-(4-Methyl-1H-imidazol-1-yl)phenyl]-2-oxo-1,4-diazaspiro[4.4]non-3-en-1-yl}-N-[3-(trifluoromethyl)phenyl]acetamide (Method B)

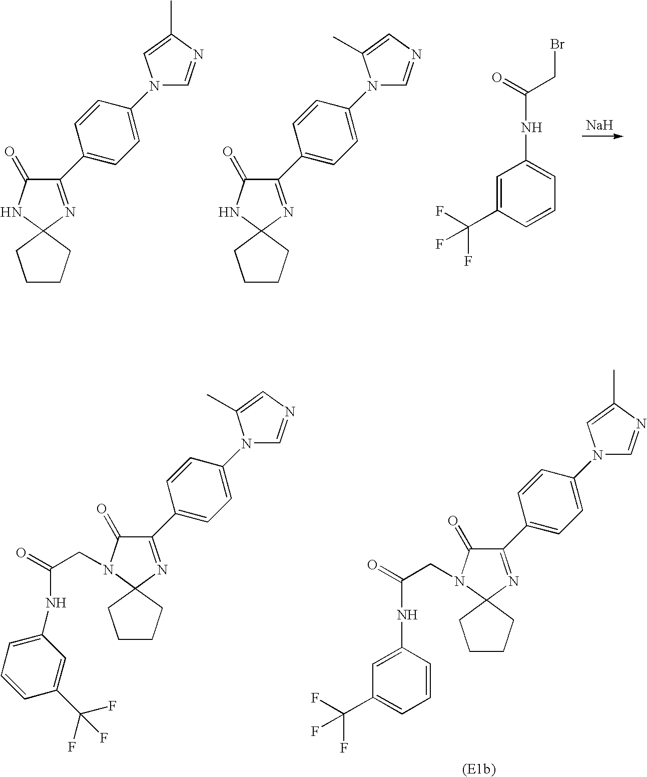

3-[4-(4-Methyl-1H-imidazol-1-yl)phenyl]-1,4-diazaspiro[4.4]non-3-en-2-one ((D4), Method B) (7.6 g) contaminated with the 5 isomer in DMF (180 ml) was cooled to ice bath temp and treated with sodium hydride (60% in oil) (1.136 g) under an atmosphere of argon. The mixture was stirred for 50 minutes when 2-bromo-N[3-(trifluoromethyl)phenyl]acetamide (D8) (7.65 g) in DMF (50 ml) was added over 2 hours by syringe pump. The mixture was stirred at ice bath temperature for 1 hour, then allowed to warm to room temp overnight. The mixture was diluted with methanol then loaded onto 2×70 g SCX cartridges, which had previously been eluted with methanol. The cartridges were washed with methanol then the compound was eluted with 2M methanolic ammonia/DCM ˜2:1. All the ammonia eluent was combined and evaporated to approx. 150 ml when a product began to precipitate. The mixture was boiled for a few minutes then cooled to ice bath temperature. The white precipitate was collected by filtration washed with ether (approx. 50 ml) and dried to give a white solid which was chromatographed using a Chiralpak IC column eluting with heptane-ethanol (20:80). The first eluting isomer was collected, the solvent was removed, the residue dissolved in refluxing methanol (approx. 750 ml) and the mixture was filtered, then reduced to approx. 250 ml when hot filtered ethanol (approx. 600 ml) was added and the volume reduced to approx. 250 ml and then allowed to cool to ice bath temperature. The white crystals were collected by filtration, washed with filtered ice cold ethanol and dried over 18 h hours at 50° C. to give the title compound E1b (6.196 g).

¹H NMR (DMSO) δ: 1.7 (2H, m), 1.9-2.1 (4H, m), 2.2 (2H, m), 2.37 (3H, s), 4.40 (2H, m), 7.45 (1H, m), 7.58 (2H, m), 7.76 (1H, m), 7.82 (2H, m), 8.10 (1H, m) 8.29 (1H, M), 8.47 (2H, s), 10.6 (1H, s).

¹⁹F NMR (DMSO) δ: −61.4

Mass Spectrum (LC/MS): Found 496 (MH⁺). Ret. time 1.85 min

### EXAMPLE 3

### 2-{3-[4-(4-methyl -1H-imidazol -1-yl)phenyl]-2-oxo-1,4-diazaspiro[4.5]dec-3-en-1-yl}-N-[3-(trifluoromethyl)phenyl]acetamide and

### EXAMPLE 4

### 2-{3-[4-(5-methyl -1H-imidazol -1-yl)phenyl]-2-oxo-1,4-diazaspiro[4.5]dec-3-en-1-yl}-N-[3-(trifluoromethyl)phenyl]acetamide

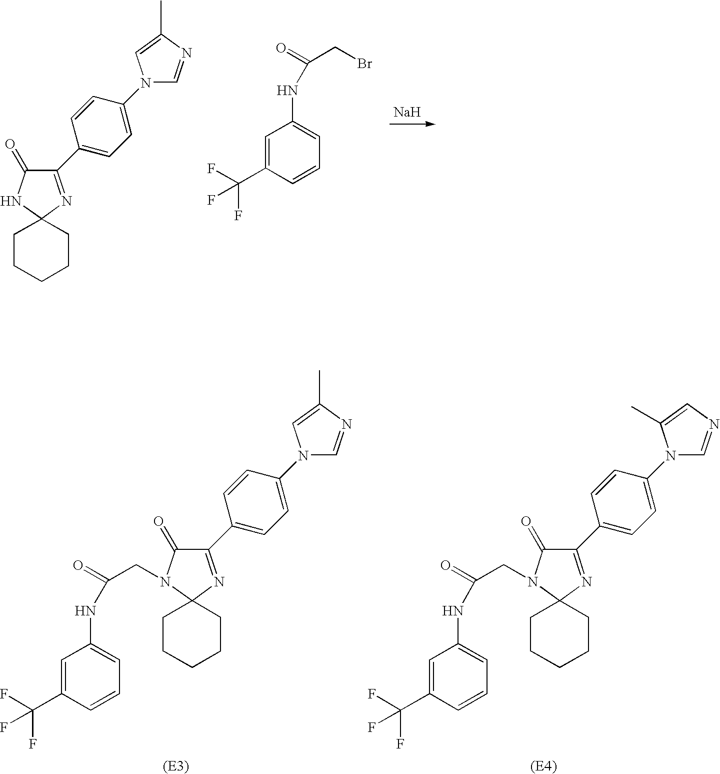

A mixture of 3-[4-(4-methyl-1H-imidazol-1-yl)phenyl]-1,4-diazaspiro[4.5]dec-3-en-2-one contaminated with 3-[4-(5-Methyl-1H-imidazol-1-yl)phenyl]-1,4-diazaspiro[4.5]dec-3-en-2-one (approx. 4:1) (D7) (150 mg) in DMF (5 ml) was cooled to ice bath temp and treated with sodium hydride (60% in oil) (23.35 mg) under an atmosphere of argon. The mixture was stirred for 30 minutes when 2-bromo-N-[3-(trifluoromethyl)phenyl]acetamide (D8) (115 mg) in DMF (2.5 ml) was added over 45 minutes by syringe pump. The mixture was then allowed to warm to room temp overnight when the solvent was partially removed. The mixture was purified by multiple MDAP's to give two isomers.

First eluting isomer was 3-[4-(5-methyl-1H-imidazol-1-yl)phenyl]-1,4-diazaspiro[4.5]dec-3-en-2-one (E4) (32 mg).

¹H NMR (CDCl₃) δ: ˜1.6 (obs, m), 1.88 (2H, m), 1.8-2.2 (6H, m), 2.20 (3H, s), 4.28 (2H, s), 6.94 (1H, m), 7.38 (1H, m), 7.4 (3H, m), 7.65 (1H, s), 7.70 (1H, m), 7.80 (1H, m), 7.89 (1H, s), 8.62 (2H, m) 9.17 (1H, m).

¹⁹F NMR (CDCl3) δ: −62.7

Mass Spectrum (LC/MS): Found 510 (MH⁺). Ret. time 2.34 min.

Second eluting isomer gave 3-[4-(4-methyl-1H-imidazol-1-yl)phenyl]-1,4-diazaspiro[4.5]dec-3-en-2-one (E3) (100 mg). ¹H NMR (CDCl₃) δ: ˜1.6 (obs, m), 1.88 (2H, m), 1.8-2.2 (6H, m), 2.28 (3H, s), 4.27 (2H, s), 7.01 (1H, m), 7.38 (1H, m), 7.48 (1H,m), 7.50 (2H, m), 7.68 (1H, m), 7.7 (1H, m), 7.82 (1H, s), 7.90 (1H, s), 8.61 (2H, m) 9.16 (1H, m).

¹⁹F NMR (CDCl3) δ: −62.7

Mass Spectrum (LC/MS): Found 510 (MH⁺). Ret. time 2.42 min.

### EXAMPLE 5

### 2-{3-[4-(1H-Imidazol-1-yl)phenyl]-2-oxo-1,4-diazaspiro[4.5]dec-3-en-1-yl}-N-[3-(trifluoromethyl)phenyl]acetamide

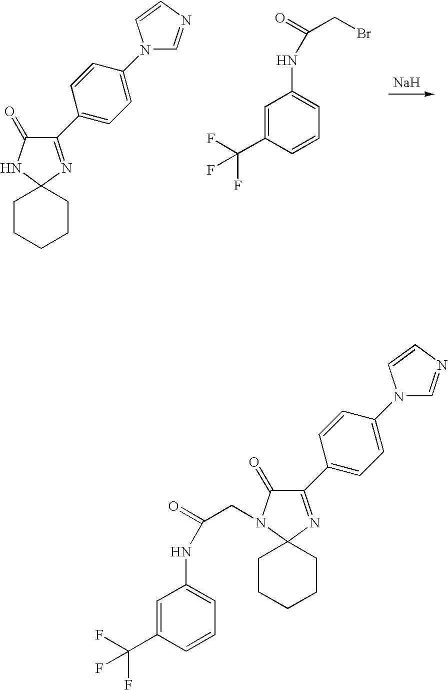

Sodium hydride (0.018 g of 60% in mineral oil) was added in two portions to a solution of 3-[4-(1H-imidazol-1-yl)phenyl]-1,4-diazaspiro[4.5]dec-3-en-2-one (D9) (0.11 g) in DMF (3 ml) stirred at ice bath temperature. The mixture was stirred for 30 minutes then 2-bromo-N-[3-(trifluoromethyl)phenyl]acetamide (D8) (0.119 g) in DMF (2.5 ml) was added by syringe pump over 45 minutes. The reaction was then allowed to warm to room temperature overnight, poured into water and extracted with ethyl acetate. The ethyl acetate layer was washed with brine, dried over sodium sulfate and the solvent was removed. The residue was chromatographed on a silica column eluted with 0-5% 2M NH₃/MeOH/DCM and the main fraction was collected. The title compound was obtained as a white crystalline solid on stirring with ether (140 mg).

¹H NMR (CDCl₃) δ: ˜1.6 (obs, m), 1.8-2.15 (8H, m), 4.28 (2H, s), 7.25 (obs, m), 7.4 (3H, m), 7.51 (2H, m), 7.67 (1H, m), 7.96 (1H, s), 8.62 (2H, m), 9.14 (1H, m).

¹⁹F NMR (CDCl3) δ: −62.7

Mass Spectrum (LC/MS): Found 496 (MH⁺). Ret. time 2.38 min.

### EXAMPLE 6

### 2-{3-[4-(2-Methyl-1H-imidazol-1-yl)phenyl]-2-oxo-1,4-diazaspiro[4.5]dec-3-en-1-yl}-N-[3-(trifluoromethyl)phenyl]acetamide

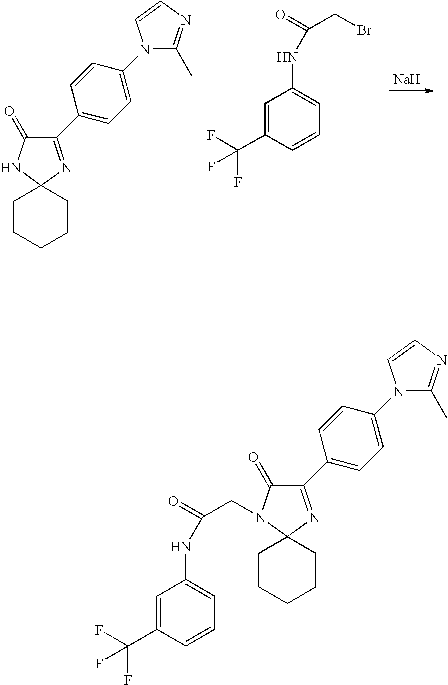

Sodium hydride (12.97 mg of 60% in mineral oil) was added in two portions to a solution of 3-[4-(2-methyl-1H-imidazol-1-yl)phenyl]-1,4-diazaspiro[4.5]dec-3-en-2-one (D10) (100 mg) in DMF (3 ml) stirred at ice bath temperature. The mixture was stirred for 30 minutes then 2-bromo-N-[3-(trifluoromethyl)phenyl]acetamide (D8) (91 mg) in DMF (2.5 ml) was added by syringe pump over 45 minutes. The reaction was then allowed to warm to room temperature overnight then poured into water and extracted with ethyl acetate. The ethyl acetate layer was washed with brine, dried over sodium sulfate and solvent removed. The residue was chromatographed on a silica column eluted with 0-5% 2M NH₃/MeOH/DCM and the main fraction was collected. A white solid was obtained on stirring with ether. This was purified by MDAP. The main peak was collected, loaded onto SCX which was then washed with methanol followed by 2M methanolic ammonia. The methanolic ammonia fraction was evaporated to give the title compound as a white solid.

¹H NMR (CDCl₃) δ: ˜1.4 (2H, m), 1.7-2.15 (8H, m), 2.42 (3H, s), 4.28 (2H, s), 7.06 (2H, m), 7.4 (4H, m), 7.67 (1H, m), 7.81 (1H, m), 8.61 (2H, m), 9.11 (1H, br).

¹⁹F NMR (CDCl3) δ: −62.7

Mass Spectrum (LC/MS): Found 510 (MH⁺). Ret. time 2.42 min.

### EXAMPLE 7

### 2-{2-Oxo-3-[4-(2-propyl-1H-imidazol-1-yl)phenyl]-1,4-diazaspiro[4.5]dec-3-en-1-yl}-N-[3-(trifluoromethyl)phenyl]acetamide hydrochloride

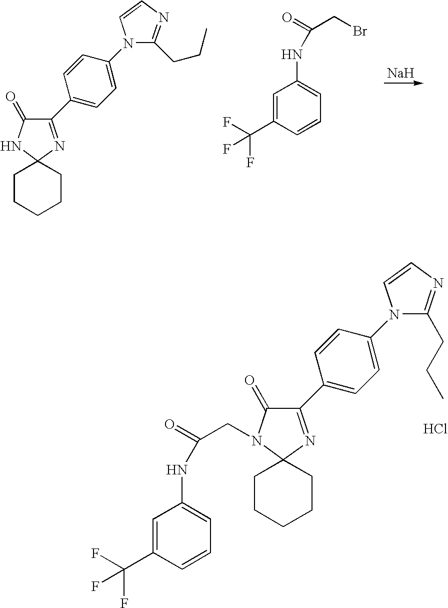

Sodium hydride (13 mg of 60% oil dispersion) was added in two portions to a solution of 3-[4-(2-propyl-1H-imidazol-1-yl)phenyl]-1,4-diazaspiro[4.5]dec-3-en-2-one (D11) (0.1 g) in DMF (3 ml) stirred at ice bath temperature. The mixture was stirred for 30 minutes then 2-bromo-N-[3-(trifluoromethyl)phenyl]acetamide (D8) (86 mg) in DMF (2.5 ml) was added by syringe pump over 45 minutes. The reaction was then allowed to warm to room temperature overnight then poured into water and extracted with ethyl acetate. The ethyl acetate layer was washed with brine, dried over sodium sulfate and solvent removed. The residue was purified by MDAP, the main fraction was collected, solvent removed and the residue was dissolved in DCM, and treated with 1M HCl in ether to give the the title compound as a white crystalline solid (79 mg).

¹H NMR (CDCl₃) δ: ˜1.3 (3H, m), 1.8-2.1 (obs, m), 3.1 (2H, m), 4.40 (2H, m), 7.1 (2H, m), 7.36 (1H, m), 7.45 (1H, m), 7.65 (2H, m), 7.95 (1H, s), 8.20 (1H, m), 8.66 (2H, m), 10.3 (1H, br).

¹⁹F NMR (CDCl3) δ: −65.2

Mass Spectrum (LC/MS): Found 538 (MH⁺). Ret. time 2.01 min.

### EXAMPLE 8

### N-(3,5-Difluorophenyl)-2-{3-[4-(4-methyl-1H-imidazol-1-yl)phenyl]-2-oxo-1,4-diazaspiro[4.4]non-3-en-1-yl}acetamide formate

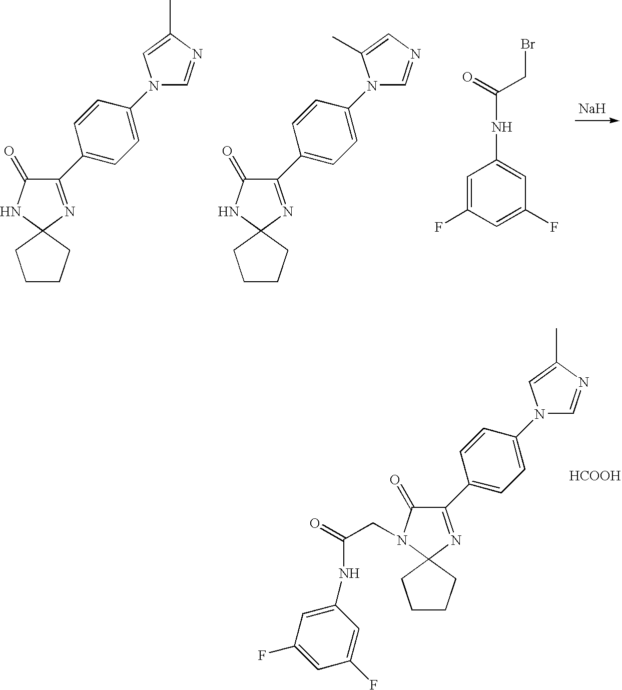

A solution of the isomeric mixture of 3-[4-(4-methyl-1H-imidazol-1-yl)phenyl]-1,4-diazaspiro[4.4]non-3-en-2-one and 3-[4-(5-methyl-1H-imidazol-1-yl)phenyl]-1,4-diazaspiro[4.4]non-3-en-2-one (approx. 4:1) ((D4) Method A) (combined mass of 100 mg) in DMF (10 ml) was cooled to 0° C. Sodium hydride (60% in mineral oil) (13.59 mg, 0.340 mmol) was added. The solution was stirred at 0° C. for 45 minutes before the slow addition (over 2 hr) of 2-bromo-N-(3,5-difluorophenyl)acetamide (D12) (85 mg) in DMF (5 ml) using a syringe pump. The resulting solution was allowed to warm to room temperature overnight, then methanol (5 ml) was added and the solution was evaporated to dryness and purified using mass directed auto-purification chromatography to give two fractions, one containing an isomeric mixture and the other containing the title compound as a white solid (42 mg).

¹H NMR (CDCl₃) δ: 1.84-1.89 (2H, m), 1.98-2.02 (assume 2H, m), 2.07-2.12 (4H, m), 2.31 (3H, s), 4.29 (2H, s), 6.49-6.53 (1H, m), 7.08-7.09 (1H, m), 7.13-7.15 (2H, m), 7.52-7.56 (2H, m), 8.39 (1H, s), 8.45 (1H, m), 8.56-6.60 (2H, m), 9.65 (1H, broad s), 9.83 (1H, s).

Mass Spectrum (Electrospray LC/MS): Found 464 (MH⁺). C₂₅H₂₃F₂N₅O₂ requires 463. Ret. time 1.63 min.

### EXAMPLE 9

### 2-{3-[4-(4-Methyl-1H-imidazol-1-yl)phenyl]-2-oxo-7-oxa-1,4-diazaspiro[4.4]non-3-en-1-yl}-N-[3-(trifluoromethyl)phenyl]acetamide

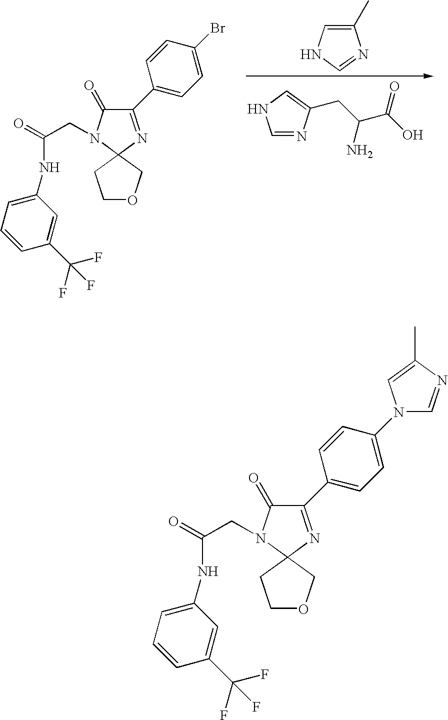

D-histidine (25 mg) and copper (I) iodide (15 mg) was stirred at 110° C. under an atmosphere of argon in DMSO (5 ml) for 30 minutes when 2-[3-(4-bromophenyl)-2-oxo-7-oxa-1,4-diazaspiro[4.4]non-3-en-1-yl]-N-[3-(trifluoromethyl)phenyl]acetamide (D14) (200 mg), 4-methylimidazole (99 mg) and potassium carbonate (111 mg) were added and heating was continued at 110° C. for 48 hours. The reaction mixture was treated with ethyl acetate (30 ml) and sodium bicarbonate solution (20 ml), stirred for 1 hour then filtered. The ethyl acetate layer was separated, dried (sodium sulphate) and the solvent was removed. The residue was purified by MDAP. The main fraction was loaded onto an SCX cartridge and the column was eluted with methanol followed by 2M ammonia in methanol. The latter was evaporated to give the title compound (17 mg).

¹H NMR (CDCl₃) δ: 2.32 (3H, s), 2.4-2.6 (2H, m), 4.05 (2H, m), 4.23 (1H, m), 4.35 (3H, m), 7.10 (1H, s), 7.36-7.55 (4H, m), 7.68 (1H, m), 7.85 (1H, m), 7.93 (1H, m), 8.6 (2H, M), 8.89 (1H, m).

¹⁹F NMR (CDCl3) δ: −62.7.

Mass Spectrum (LC/MS): Found 498 (MH⁺). Ret. time 1.62 min.

### EXAMPLE 10

### 2-{3-[4-(4-Methyl-1H-imidazol-1-yl)phenyl]-2-oxo-7-oxa-1,4-diazaspiro[4.5]dec-3-en-1-yl}-N-[3-(trifluoromethyl)phenyl]acetamide and 2-{3-[4-(5-methyl-1H-imidazol-1-yl)phenyl]-2-oxo-7-oxa-1,4-diazaspiro[4.5]dec-3-en-1-yl}-N-[3-(trifluoromethyl)phenyl]acetamide (Approx. 4:1 Mixture)

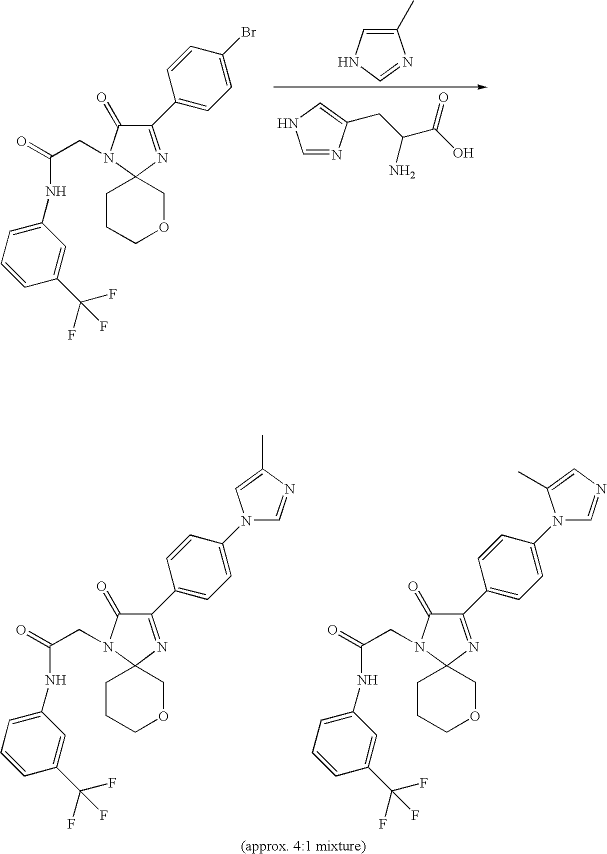

Copper (I) iodide (2.1 mg) and D-histidine (3.4 mg) in DMSO (2 ml) was heated under argon for 40 minutes at 110° C. when 2-[3-(4-bromophenyl)-2-oxo-7-oxa-1,4-diazaspiro[4.5]dec-3-en-1-yl]-N-[3-(trifluoromethyl)phenyl]acetamide (D16) (57 mg), 4-methylimidazole (18.3 mg) and potassium carbonate (30.9 mg) were added and heating was continued at 110° C. for 48 hours. The cooled reaction mixture was treated with mixture of sodium bicarbonate solution and ethyl acetate and the resulting mixture was stirred for 1 hour, filtered and the filtrate was separated. The ethyl acetate layer was dried over sodium sulphate, evaporated and the residue was purified by MDAP. The main fraction was loaded onto an SCX cartridge and the column was eluted with methanol followed by 2M ammonia in methanol. The later was evaporated to give the title compound being an approximately 4:1 mixture of 2-{3-[4-(4-methyl-1H-imidazol-1-yl)phenyl]-2-oxo-7-oxa-1,4-diazaspiro[4.5]dec-3-en-1-yl}-N-[3-(trifluoromethyl)phenyl]acetamide and 2-{3-[4-(5-methyl-1H-imidazol-1-yl)phenyl]-2-oxo-7-oxa-1,4-diazaspiro[4.5]dec-3-en-1-yl}-N-[3-(trifluoromethyl)phenyl]acetamide by NMR (7 mg).

Mass Spectrum (LC/MS): Found 512 (MH⁺). Ret. time 1.64 min.

¹H NMR (CDCl₃) δ: 6.95 (0.2H, s), 7.10 (0.8H, s). Key peaks only for determination of product ratio; remainder of spectrum very complex.

¹⁹F NMR (CDCl₃) δ: −62.7 and −62.8.

### EXAMPLE 10a

### CHIRAL ISOMER 1

### 2-{3-[4-(4-methyl-1H-imidazol-1-yl)phenyl]-2-oxo-7-oxa-1,4-diazaspiro[4.5]dec-3-en-1-yl}-N-[3-(trifluoromethyl)phenyl]acetamide hydrochloride

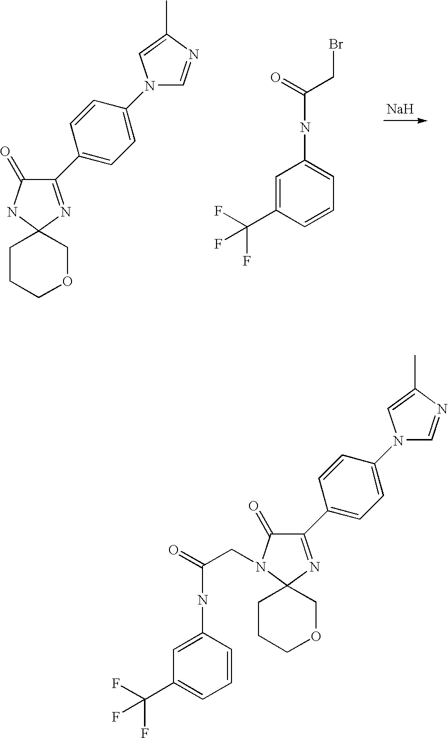

3-[4-(4-Methyl-1H-imidazol-1-yl)phenyl]-7-oxa-1,4-diazaspiro[4.5]dec-3-en-2-one (D31a), isomer 1 (94 mg) in DMF (6 ml) was cooled to ice bath temp and treated with sodium hydride 60% in oil (13 mg) under an atmosphere of argon. The mixture was stirred for 20 minutes when 2-bromo-N-[3-(trifluoromethyl)phenyl]acetamide (85 mg) in DMF (2.5 ml) was added over 1.5 hours by syringe pump. The mixture was then allowed to warm to room temp overnight. The solvent was partially removed and the residue was purified by low pH MDAP. The fractions were loaded onto SCX and the free base was eluted with 2M methanolic ammonia. The solvent was removed and the residue was dissolved in DCM, treated with HCl-Et₂O, solvent removed and a white solid was obtained from ether (78 mg).

¹H NMR (DMSO) δ: 1.75 (2H, m), 2.05-2.35 (obs, m), 2.45 (3H, s), 3.4-3.6 (obs, m), 3.9 (2H, m), 4.98 (2H, m), 7.45 (1H, m), 7.58 (1H, m), 7.75 (1H, m), 7.98 (2H, m), 8.12 (2H, m), 8.58 (2H, m), 9.63 (1H, s), 10.74 (1H, s).

¹⁹F NMR (DMSO) δ: 61.4

Mass Spectrum (LC/MS): Found 512 (MH⁺). Ret. time 1.63 min.

### EXAMPLE 10b

### CHIRAL ISOMER 2

### 2-{3-[4-(4-methyl-1H-imidazol-1-yl)phenyl]-2-oxo-7-oxa-1,4-diazaspiro[4.5]dec-3-en-1-yl}-N-[3-(trifluoromethyl)phenyl]acetamide

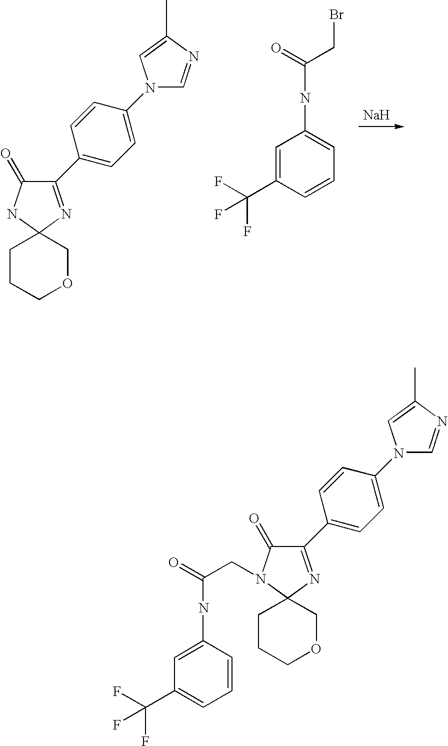

3-[4-(4-Methyl-1H-imidazol-1-yl)phenyl]-7-oxa-1,4-diazaspiro[4.5]dec-3-en-2-one (D31b) isomer 2 (94 mg) in DMF (6 ml) was cooled to ice bath temp and treated with sodium hydride 60% in oil (12 mg) under an atmosphere of argon. The mixture was stirred for 20 minutes when 2-bromo-N-[3-(trifluoromethyl)phenyl]acetamide (85 mg) in DMF (2.5 ml) was added over 1.5 hours by syringe pump. The mixture was then allowed to warm to room temp overnight. The mixture was poured into water and extracted with dichloromethane. The organic layer was washed with brine and then dried with hydromatrix and the solvent was removed to give the title compound (104 mg).

¹H NMR (CDCl₃) δ: 1.7 (obs, m), 2.0 (2H, m), 2.15-2.4 (2H, m), 3.31 (3H, m), 3.71 (1H, m), 3.85 (2H, m), 4.05 (1H, m), 4.45 (2H, m), 7.10 (1H, s), 7.3-7.5 (4H, m), 7.68 (1H, m), 7.84 (1H, m), 7.94 (1H, m), 8.60 (2H, m), 9.09 (1H, s).

¹⁹F NMR (DMSO) δ: 62.7

Mass Spectrum (LC/MS): Found 512 (MH⁺). Ret. time 1.62 min.

### EXAMPLE 11

### 2-{2-Oxo-3-[4-(2-pyridinyl)phenyl]-7-oxa-1,4-diazaspiro[4.4]non-3-en-1-yl}-N-[3-(trifluoromethyl)phenyl]acetamide hydrochloride

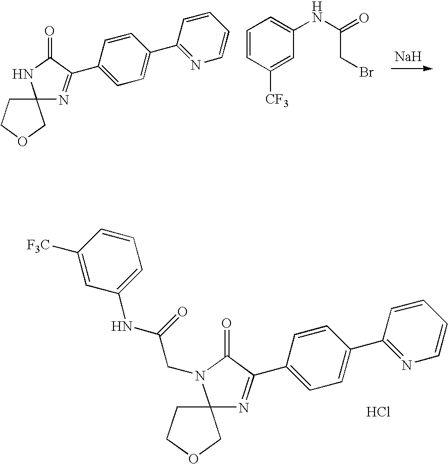

Sodium hydride (18.00 mg of 60% in mineral oil, 0.450 mmol) was added to a stirring, ice-bath cooled solution of 3-[4-(2-pyridinyl)phenyl]-7-oxa-1,4-diazaspiro[4.4]non-3-en-2-one (D17) (110 mg) in DMF (4 ml) under argon. After stirring for 30 min. a solution of 2-bromo-N-[3-(trifluoromethyl)phenyl]acetamide (D8) (106 mg, 0.375 mmol) in DMF (2 ml) was added over 1 hour using a syringe pump, maintaining an ice-bath temperature. The resulting reaction mixture was allowed to come to room temperature then stirred for a further 5 hours. The reaction mixture was diluted with MeOH (15 ml) and passed through an SCX column (2 g). After washing with MeOH (30 ml) the partially purified product was eluted with 2M NH₃-MeOH (5 ml). Evaporation afforded a yellow solid which was purified by MDAP. Passing the appropriate fractions through an SCX column, washing with MeOH then eluting with 2M NH₃-MeOH afforded 2-{2-oxo-3-[4-(2-pyridinyl)phenyl]-7-oxa-1,4-diazaspiro[4.4]non-3-en-1-yl}-N-[3-(trifluoromethyl)phenyl]acetamide (86 mg). The free-base in DCM (2 ml) was treated with an excess of 1M HCl in diethyl ether (0.870 ml). The volatiles were removed in vacuo and the residue dried at high vacuum at 50° C. to afford the title compound as a cream solid (90 mg).

¹H NMR (CDCl₃) δ: 2.30 (1H, m), 2.51 (1H, m obscured by DMSO), 3.80 (1H, d), 4.00 (1H, d), 4.16 (2H, m), 4.45 (2H, m), 7.44 (1H, d), 7.51 (1H, m), 7.59 (1H, m), 7.77 (1H, d), 8.04 (1H, m), 8.14 (2H, m), 8.28 (2H, m), 8.51 (2H, m), 8.75 (1H, m), 10.70 (1H, s).

Mass Spectrum (LC/MS): Found 495 (MH⁺). Ret. time 2.54 min.

### EXAMPLE 12

### 2-{2-Oxo-3-[4-(2-pyridinyl)phenyl]-1,4-diazaspiro[4.4]non-3-en-1-yl}-N-[3-(trifluoromethyl)phenyl]acetamide hydrochloride

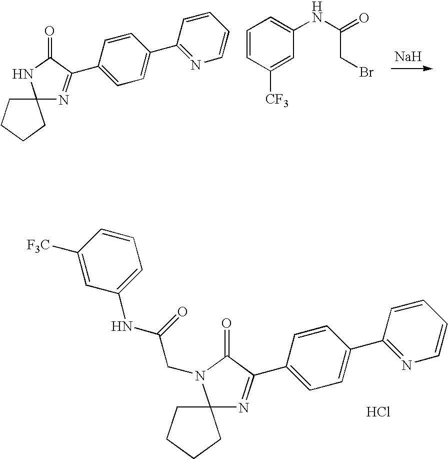

Sodium hydride (24.71 mg of 60% in mineral oil, 0.618 mmol) was added to a, stirring, ice-bath cooled solution of 3-[4-(2-pyridinyl)phenyl]-1,4-diazaspiro[4.4]non-3-en-2-one (D18) (150 mg, 0.515 mmol) in DMF (4 ml) under argon. After stirring for 30 min. a solution of 2-bromo-N-[3-(trifluoromethyl)phenyl]acetamide (D8) (145 mg, 0.515 mmol) in DMF (2 ml) was added over 1 hour using a syringe pump, maintaining an ice-bath temperature. The resulting reaction mixture was allowed to come to room temperature then stirred for a further 14 hours. The reaction mixture was diluted with MeOH (15 ml) and passed through an SCX column (2 g). After washing with MeOH (30 ml) the partially purified product was eluted with 2M NH₃-MeOH (5 ml). Evaporation afforded a yellow solid which was contaminated with approx 6% bis-alkylated material (by LCMS). The solid was triturated with diethyl ether to afford 2-{2-oxo-3-[4-(2-pyridinyl)phenyl]-1,4-diazaspiro[4.4]non-3-en-1-yl}-N-[3-(trifluoromethyl)phenyl]acetamide (155 mg, 61.1%). The free-base in DCM (2 ml) was treated with an excess of 1M HCl in diethyl ether (1.574 ml, 1.574 mmol). The volatiles were removed in vacuo and the residue dried to afford the title compound as a cream solid (150 mg).

¹H NMR (CDCl₃) δ: 1.70 (2H, m), 2.00 (4H,bm), 2.15 (2H, m), 4.39 (2H, s), 7.43 (1H, d), 7.52 (1H, m), 7.59 (1H, m), 7.77 (1H, m), 8.06 (1H, m), 8.15 (2H, m), 8.26 (2H, d), 8.49 (2H, d), 8.75 (1H, m), 10.70 (1H, s).

Mass Spectrum (LC/MS): Found 493 (MH⁺). Ret. time 2.81 min.

### EXAMPLE 13

### N-(3-Chlorophenyl)-2-{3-[4-(4-methyl-1H-imidazol-1-yl)phenyl]-2-oxo-1,4-diazaspiro[4.4]non-3-en-1-yl}acetamide

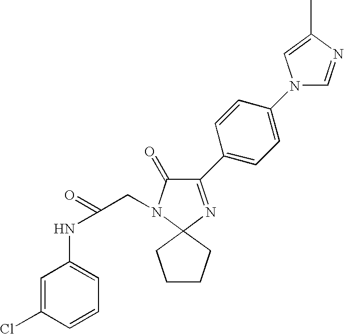

A solution of the isomeric mixture of 3-[4-(4-methyl-1H-imidazol-1-yl)phenyl]-1,4-diazaspiro[4.4]non-3-en-2-one and 3-[4-(5-methyl-1H-imidazol-1-yl)phenyl]-1,4-diazaspiro[4.4]non-3-en-2-one (approx. 4:1) ((D4) Method A) (combined mass of 100 mg) in DMF (5 ml) was cooled to 0° C. Sodium hydride (13.59 mg of 60% in mineral oil, 0.340 mmol) was added and the solution was stirred at 0° C. for 45 minutes before the slow addition (2 hr) of 2-bromo-N-(3-chlorophenyl)acetamide (D25) (84 mg) in DMF (2 ml) using a syringe pump. The resulting solution was allowed to warm to room temperature overnight. Methanol was then added and the solution evaporated to dryness then re-dissolved in DCM. Water and sodium bicarbonate solution were added. The resulting mixture was poured through a phase separating cartridge and evaporated to dryness, then purified using mass directed auto-purification chromatography to give two fractions, one containing an isomeric mixture and the other containing the title compound as a white solid (36 mg).

¹H NMR (D₆-DMSO) δ: 1.65-1.70 (2H, m), 1.90-2.01 (4H, m), 2.09-2.14 (2H, m), 2.17 (3H, s), 4.34 (2H, s), 7.12-7.15 (1H, m), 7.34-7.38 (1H, t), 7.43-7.46 (1H, m), 7.57 (1H, s), (7.79-7.81 (3H, m), 8.28 (1H, s), 8.43-8.46 (2H, m), 10.40 (1H, s). Mass Spectrum (Electrospray LC/MS): Found 462 (MH⁺). C₂₅H₂₄ClN₅O₂ requires 461. Ret. time 1.67 min.

### EXAMPLE 14

### 2-{2-Oxo-3-[3-(2-pyridinyl)phenyl]-1,4-diazaspiro[4.5]dec-3-en-1-yl}-N-[3-(trifluoromethyl)phenyl]acetamide

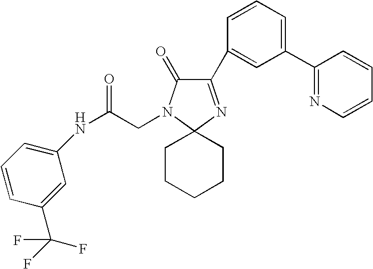

A solution of 3-[3-(2-pyridinyl)phenyl]-1,4-diazaspiro[4.5]dec-3-en-2-one (D24) (21 mg) in DMF (4 ml) was cooled to 0° C. Sodium hydride (2.75 mg of 60% in mineral oil, 0.069 mmol) was added. The solution was stirred at 0° C. for 45 minutes before the slow addition (1 hr) of 2-bromo-N-[3-(trifluoromethyl)phenyl]acetamide (D8) (19.40 mg) in DMF (1 ml) using a syringe pump. The resulting solution was allowed to warm to room temperature overnight and stirred for another 18 hours. The solution was then cooled again whilst stirring to 0° C. and sodium hydride (2.75 mg, 0.069 mmol) was added. Stirring continued for 45 minutes before the slow addition (1 hr) of 2-bromo-N-[3-(trifluoromethyl)phenyl]acetamide (D8) (19.40 mg) in DMF (1 ml) using a syringe pump. The resulting solution was allowed to warm to room temperature and stirred over the weekend, then methanol (5 ml) was added and the solution was evaporated to dryness, then re-dissolved in DCM (5 ml) and water (5 ml) was added. The resulting solution was poured through a phase separating cartridge and evaporated to dryness Purification using mass directed auto-purification chromatography gave the title compound as a white solid (22 mg).

¹H NMR (CD₃OD) δ: 1.39-1.51 (3H, m), 1.81-1.91 (3H, m), 1.98-2.10 (assume 4H, m), 4.39 (2H, s), 7.37-7.41 (2H, m), 7.48-7.52 (1H, t), 7.61-7.65 (1H, t), 7.56-7.77 (1H, d), 7.92-7.93 (2H, m), 8.01 (1H, s), 8.12-8.15 (2H, m), 8.48-8.50 (1H, m), 8.63-8.97 (1H, m), 8.98 (1H, s). Mass Spectrum (Electrospray LC/MS): Found 507 (MH⁺). C₂₈H₂₅F₃N₄O₂ requires 506. Ret. time 3.14 min.

### EXAMPLE 15

### 2-{2-Oxo-3-[4-(2-pyridinyl)phenyl]-7-oxa-1,4-diazaspiro[4.5]dec-3-en-1-yl}-N-[3-(trifluoromethyl)phenyl]acetamide hydrochloride (racemic)

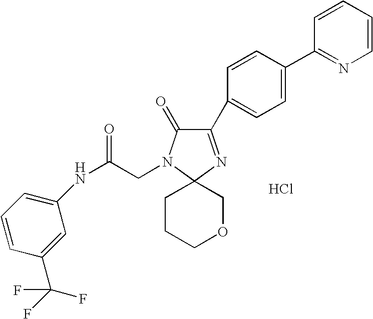

Sodium hydride (60% in mineral oil, 22.33 mg, 0.558 mmol) was added to a, stirring, ice-bath cooled solution of 3-[4-(2-pyridinyl)phenyl]-7-oxa-1,4-diazaspiro[4.5]dec-3-en-2-one (D26) (143 mg) in DMF (4 ml) under argon. After stirring for 30 min. a solution of 2-bromo-N-[3-(trifluoromethyl)phenyl]acetamide (D8) (131 mg) in DMF (2 ml) was added over 1 h using a syringe pump, maintaining an ice-bath temperature. The resulting reaction mixture was allowed to come to room temperature then stirred for a further 5 h. LCMS indicated the reaction had gone to completion. The reaction mixture was diluted with MeOH (15 ml) and passed through an SCX column (2 g). After eluting with MeOH (30 ml) the partially purified product was eluted with 2M NH₃-MeOH (5 ml). Evaporation afforded a cream solid which was purified by MDAP (extended retention time). Passing the appropriate fractions through an SCX column, washing with MeOH then eluting with 2M NH₃-MeOH afforded the free-base of 2-{2-oxo-3-[4-(2-pyridinyl)phenyl]-7-oxa-1,4-diazaspiro[4.5]dec-3-en-1-yl}-N-[3-(trifluoromethyl)phenyl]-acetamide as a white foam (160 mg).

¹H NMR (CDCl₃) δ: 2.00 (2H, m), 2.10-2.35 (2H, m), 3.70-3.90 (3H, m), 4.01 (1H, m), 4.48 (2H, ABq), 7.26-7.40 (3H, m and CHCl₃), 7.62 (1H, m), 7.81 (3H, m), 8.13 (2H, m), 8.59 (2H, m), 8.73 (1H, m), 9.04 (1H, s).

Mass Spectrum (Electrospray LC/MS): Found 509 (MH⁺). C₂₇H₂₃F₃N₄O₃ requires 508. Ret. time 2.55 min.

A solution of the free base (155 mg) in DCM (2 ml) was treated with an excess of 1M HCl in diethyl ether (1.524 ml, 1.524 mmol). The volatiles were removed in vacuo and the residue dried under high vacuum at 50° C. to give the title compound (hydrochloride salt) as a cream solid.

Mass Spectrum (Electrospray LC/MS): Found 509 (MH⁺). C₂₇H₂₃F₃N₄O₃ requires 508. Ret. time 2.51 min.

### EXAMPLE 15a AND EXAMPLE 15b

### Enantiomers of 2-{2-Oxo-3-[4-(2-pyridinyl)phenyl]-7-oxa-1,4-diazaspiro[4.5]dec-3-en-1-yl}-N-[3-(trifluoromethyl)phenyl]acetamide hydrochloride

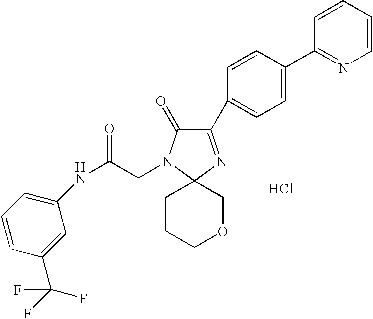

2-{2-Oxo-3-[4-(2-pyridinyl)phenyl]-7-oxa-1,4-diazaspiro[4.5]dec-3-en-1-yl}-N-[3-(trifluoromethyl)phenyl]acetamide hydrochloride (E15; 75 mg) was dissolved in methanol and applied to an SCX column. Elution with 2M NH₃-MeOH afforded the free-base which was dried in vacuo. Chiral separation (Chiralpak IC 250 mm×4.6 mm, 5 μm particle size, serial no. IC00CE-LI004; heptane: absolute EtOH, 80:20 v/v pump-mixed, isocratic for 30 min.; ambient temperature; UV absorbance at 215 nm; 10 μl injection volume) afforded the separated enantiomers with retention times of 12.5 and 14.3 min. A solution of the faster running isomer (18 mg) in DCM (2 ml) was treated with an excess of 1M HCl in diethyl ether (0.177 ml, 0.177 mmol). The volatiles were removed in vacuo and the residue dried under high vacuum at 50° C. to afford E15a.

Mass Spectrum (Electrospray LC/MS): Found 509 (MH⁺). C₂₇H₂₃F₃N₄O₃ requires 508. Ret. time 2.53 min.

A solution of the slower eluting enantiomer (19 mg, 0.037 mmol) in DCM (2 ml) was treated with an excess of 1M HCl in diethyl ether (0.187 ml, 0.187 mmol). The volatiles were removed in vacuo and the residue dried under high vacuum at 50° C. to afford E15b.

Mass Spectrum (Electrospray LC/MS): Found 509 (MH⁺). C₂₇H₂₃F₃N₄O₃ requires 508. Ret. time 2.54 min.

### EXAMPLE 16

### N-(3,5-Difluorophenyl)-2-{2-oxo-3-[4-(2-pyridinyl)phenyl]-7-oxa-1,4-diazaspiro[4.5]dec-3-en-1-yl}acetamide hydrochloride

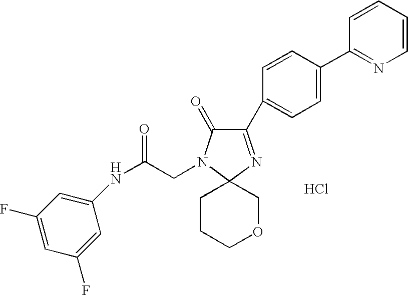

Sodium hydride (60% in mineral oil, 22.33 mg, 0.558 mmol) was added to a, stirring, ice-bath cooled solution of 3-[4-(2-pyridinyl)phenyl]-7-oxa-1,4-diazaspiro[4.5]dec-3-en-2-one (D26) (143 mg) in DMF (4 ml) under argon. After stirring for 30 min. a solution of 2-bromo-N-(3,5-difluorophenyl)acetamide (D12) (116 mg) in DMF (2 ml) was added over 1 h using a syringe pump maintaining an ice-bath temperature. The resulting reaction mixture was allowed to come to room temperature then stirred for a further 3 h. The reaction mixture was diluted with MeOH (15 ml) and passed through an SCX column (2 g). After eluting with MeOH (30 ml) the partially purified product was eluted with 2M NH₃-MeOH (5 ml). Evaporation afforded a cream solid which was purified by MDAP (extended retention time). Passing the appropriate fractions through an SCX column, washing with MeOH then eluting with 2M NH₃-MeOH afforded the free base of N-(3,5-difluorophenyl)-2-{2-oxo-3-[4-(2-pyridinyl)phenyl]-7-oxa-1,4-diazaspiro[4.5]dec-3-en-1-yl}acetamide (150 mg).

¹H NMR (CDCl₃) δ: 1.95-2.29 (4H, bm), 3.79 (3H, m), 3.98 (1H, m), 4.47 (2H, ABq), 6.53 (1H, m), 7.11 (2H, m), 7.29 (1H, m, obscured by CHCl₃), 7.80 (2H, m), 8.13 (2H, m), 8.58 (2H, m), 8.74 (1H, d), 9.10 (1H, s).

Mass Spectrum (Electrospray LC/MS): Found 477 (MH⁺). C₂₆H₂₂F₂N₄O₃ requires 476. Ret. time 2.39 min.

A solution of the free base (150 mg) in DCM (2 ml) was treated with an excess of 1M HCl in diethyl ether (1.574 ml, 1.574 mmol). The volatiles were removed in vacuo and the residue dried under high vacuum at 50° C. to afford the title hydrochloride as a cream solid.

Mass Spectrum (Electrospray LC/MS): Found 477 (MH⁺). C₂₆H₂₂F₂N₄O₃ requires 476. Ret. time 2.35 min.

### EXAMPLE 17

### 2-{3-[4-(2-Methyl-1H-imidazol-1-yl)phenyl]-2-oxo-1,4-diazaspiro[4.4]non-3-en-1-yl}-N-[3-(trifluoromethyl)phenyl]acetamide hydrochloride

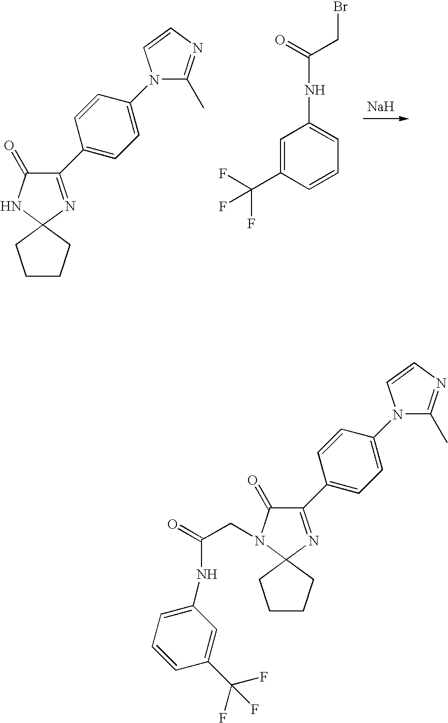

3-[4-(2-Methyl-1H-imidazol-1-yl)phenyl]-1,4-diazaspiro[4.4]non-3-en-2-one (D30) (62 mg) in DMF (5 ml) was cooled to ice bath temp and treated with sodium hydride 60% in oil (10 mg) under an atmosphere of argon. The mixture was stirred for 30 minutes when 2-bromo-N-[3-(trifluoromethyl)phenyl]acetamide (D8) (65.4 mg) in DMF (1 ml) was added over 1 hour by syringe pump, then allowed to warm to room temperature overnight. The reaction had only gone to approx. 60%, therefore it was re-cooled and the cooled mixture treated with sodium hydride (60% in oil) (7 mg) under an atmosphere of argon. The mixture was stirred for 30 minutes when additional 2-bromo-N-[3-(trifluoromethyl)phenyl]acetamide (50 mg) in DMF (1 ml) was added over 1 hour by syringe pump, then mixture was stirred for 2 hours. The mixture was poured into water and extracted with ethyl acetate. The organic layers were dried with hydromatrix and the solvent was removed and the combined residue was purified by multiple high pH MDAP. The main fractions were combined, evaporated and taken up in DCM, then treated with 1M HCl in ether and the solvent was removed. A white crystalline solid was obtained on stirring with ether which was collected by filtration to give the title compound (155 mg).

Mass Spectrum (LC/MS): Found 496 (MH⁺). Ret. time 1.73 min.

¹H NMR (d⁶DMSO) δ: 1.7 (2H, m), 1.85-2.1 (4H, m), 2.2 (2H, m), 2.6 (obs, s), 4.41 (2H, s), 6.95 (1H, m), 7.7-7.85 (4H, m), 7.96 (1H, m), 8.13 (1H, m), 8.56 (2H, m), 10.75 (1H, s), 14.7 (1H, br).

¹⁹F NMR (CDCl₃) δ: 61,4

### EXAMPLE 18

### 2-{3-[4-(2,4-Dimethyl-1H-imidazol-1-yl)phenyl]-2-oxo-1,4-diazaspiro[4.4]non-3-en-1-yl}-N-[3-(trifluoromethypphenyl]acetamide hydrochloride

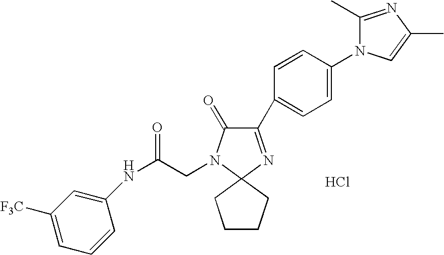

Sodium hydride in mineral oil (46.4 mg, 1.160 mmol) was added to a stirring, ice-bath cooled solution of 3-[4-(2,4-dimethyl-1H-imidazol-1-yl)phenyl]-1,4-diazaspiro[4.4]non-3-en-2-one (D27) (265 mg, 0.773 mmol) in DMF (6 ml) under argon. After stirring for 30 min. a solution of 2-bromo-N-[3-(trifluoromethyl)phenyl]acetamide (D8) (218 mg, 0.773 mmol) in DMF (2.5 ml) was added over 1 h using a syringe pump maintaining an ice-bath temperature. The resulting reaction mixture was allowed to come to room temperature then stirred for a further 4 h. LCMS indicated the reaction had gone almost to completion. The reaction mixture was diluted with MeOH (15 ml) and passed through an SCX column (10 g). After eluting with MeOH (70 ml) the partially purified product was eluted with 2M NH₃-MeOH (20 ml). Evaporation afforded a brown oil (400 mg). A 100 mg aliquot was purified by MDAP (extended retention time). Passing the appropriate fractions through an SCX column, washing with MeOH then eluting with 2M NH₃-MeOH afforded the free-base of the title compound as an off-white foam (40 mg).

¹H NMR (CDCl₃) δ: 1.80-2.20 (8H, bm), 2.26 (3H, s), 2.40 (3H, s), 4.30 (2H, s), 6.78 (1H, d), 7.40 (4H, m), 7.68 (1H, d), 7.83 (1H, s), 8.56 (2H, m), 9.18 (1H, s).

Mass Spectrum (Electrospray LC/MS): Found 510 (MH⁺). C₂₇H₂₆F₃N₅O₃ requires 509. Ret. time 1.68 min.

The remaining 300 mg crude was similarly purified by MDAP to afford additional product (110 mg, white powder) and product contaminated with a regioisomeric impurity carried through from D27 (110 mg foamed white solid).

A solution of the free-base (35 mg from the first purification) in DCM (2 ml) was treated with an excess of 1M HCl in diethyl ether (0.206 ml, 0.206 mmol). The volatiles were removed in vacuo and the residue dried under high vacuum at 55° C. to afford the title compound as a cream solid (37 mg).

Mass Spectrum (Electrospray LC/MS): Found 510 (MH⁺). C₂₇H₂₆F₃N₅O₂ requires 509. Ret. time 1.79 min.

### EXAMPLE 19

### 2-{3-[4-(4,5-Dimethyl-1H-imidazol-1-yl)phenyl]-2-oxo-1,4-diazaspiro[4.4]non-3-en-1-yl}-N-[3-(trifluoromethyl)phenyl]acetamide hydrochloride

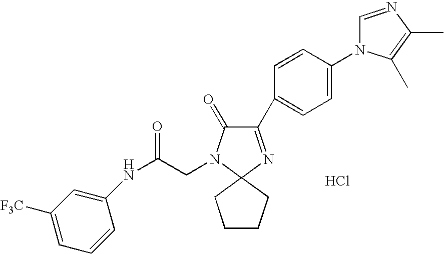

Sodium hydride in mineral oil (21.40 mg, 0.535 mmol) was added to a, stirring, ice-bath cooled solution of 3-[4-(4,5-dimethyl-1H-imidazol-1-yl)phenyl]-1,4-diazaspiro[4.4]non-3-en-2-one (D28) (150 mg, 0.486 mmol) in DMF (5 ml) under argon. After stirring for 30 min. a solution of 2-bromo-N-[3-(trifluoromethyl)phenyl]acetamide (D8) (137 mg) in DMF (3 ml) was added over 1 h using a syringe pump maintaining an ice-bath temperature. The resulting reaction mixture was allowed to come to room temperature then stirred for a further 4 h. The reation mixture was diluted with methanol (20 ml) and applied to an SCX column (10 g). After washing with MeOH (50 ml) the product was eluted with 2M NH₃-MeOH. Evaporation of the solvent afforded a brown gum. On dissolving in 1:1 MeOH:DMSO for MDAP purification a small amount of white solid precipitated. This was collected, washed with diethyl ether and dried The material was reapplied to an SCX column (1 g) in MeOH (10 ml), washing (30 ml MeOH) and eluting as described above to remove residual DMSO. Drying afforded the free-base of the title compound as a white solid (20 mg).

¹H NMR (CDCl₃) δ: 1.80-2.15 (8H, bm), 2.17 (3H, s, overlapping with adjacent bm), 2.25 (3H, s), 4.30 (2H, s), 7.40 (4H, m), 7.58 (1H, s), 7.68 (1H, d), 7.84 (1H, s), 8.57 (2H, d), 9.32 (1H, s).

Mass Spectrum (Electrospray LC/MS): Found 510 (MH⁺). C₂₇H₂₆F₃N₅O₃ requires 509. Ret. time 1.81 min.

A solution of the free-base (20 mg) in DCM (2 ml) was treated with an excess of 1M HCl in diethyl ether (0.12 ml, 0.120 mmol). The volatiles were removed in vacuo and the residue dried under high vacuum at 55° C. to afford the title hydrochloride as a pale yellow solid (20 mg).

Mass Spectrum (Electrospray LC/MS): Found 510 (MH⁺). C₂₇H₂₆F₃N₅O₃ requires 509. Ret. time 1.77 min.

### EXAMPLE 20

### 2-{3-[3-(4-Methyl-1H-imidazol-1-yl)phenyl]-2-oxo-1,4-diazaspiro[4.5]dec-3-en-1-yl}-N-[3-(trifluoromethyl)phenyl]acetamide hydrochloride and

### EXAMPLE 21

### 2-{3-[3-(5-Methyl-1H-imidazol-1-yl)phenyl]-2-oxo-1,4-diazaspiro[4.5]dec-3-en-1-yl}-N[3-(trifluoromethyl)phenyl]acetamide hydrochloride

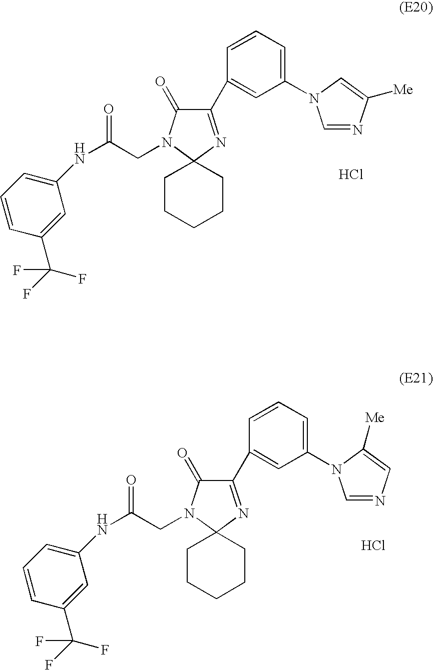

A stirred solution of D29 (70 mg), a mixture of approx. 65% of 3-[3-(4-methyl-1H-imidazol-1-yl)phenyl]-1,4-diazaspiro[4.5]dec-3-en-2-one; 15% of 3-[3-(5-methyl-1H-imidazol-1-yl)phenyl]-1,4-diazaspiro[4.5]dec-3-en-2-one and 20% impurities, in DMF (3 ml) at 0° C. under argon was treated with sodium hydride (9.99 mg of 60% oil dispersion; 0.250 mmol) and maintained for 15 minutes, then allowed to warm to room temperature and stir for 25 minutes. The mixture was re-cooled to 0° C. and treated dropwise over 1 hour via syringe pump with a solution of 2-bromo-N-[3-(trifluoromethyl)phenyl]acetamide (D8) (77 mg) in DMF (2 ml). The reaction mixture was then allowed to warm to room temperature and stir over a weekend, then concentrated under vacuum. The residue was treated with 10% Na₂CO₃ solution (10 ml) and extracted with EtOAc (2×15 ml). The combined extract was dried (Na₂SO₄) and concentrated under vacuum to leave an orange oil, which was purified by MDAP to give a good separation of the two methyl imidazole isomers.

The first eluting component was the 5-methylimidazole isomer, containing approx. 5% of the 4-isomer, obtained as a colourless oil (8 mg). A solution of this material in dichloromethane (2 ml) was treated with 1M HCl in ether (0.031 ml, 0.031 mmol) and the resulting mixture concentrated under a flow of air followed by drying under vacuum at 50° C. overnight to afford the title compound E21 as a white solid (9 mg). This contained approx. 5% of the corresponding 4-methylimidazole isomer.

Mass Spectrum (Electrospray LC/MS): Found 510.2 (MH⁺). C₂₇H₂₆F₃N₅O₂ requires 509. Ret. time 1.87 min.

¹H NMR (d⁶DMSO) δ: 1.30-1.45 (3H, m), 1.72-1.85 (3H, m), 1.85-1.98 (2H, m), 1.98-2.12 (2H, m), 2.22 (3H, s), 4.41 (2H, s), 7.36 (1H, d), 7.57 (1H, t), 7.63 (1H, s), 7.77 (1H, d), 7.80-7.93 (2H, m), 8.12 (1H, s), 8.53 (1H, s), 8.61 (1H, d), 9.37 (1H, s), 10.72 (1H, s).

The second eluting component was the 4-methylimidazole isomer, containing approx. 6% of the 5-isomer, obtained as a white semi-solid. (22 mg). A solution of this material in dichloromethane (2 ml) was treated with 1M HCl in ether (0.086 ml, 0.086 mmol) and the resulting mixture concentrated under a flow of air followed by drying under vacuum at 50° C. overnight to afford the title compound E20 as a white solid (24 mg). This contained approx. 6% of the corresponding 5-methylimidazole isomer.

Mass Spectrum (Electrospray LC/MS): Found 510.2 (MH⁺). C₂₇H₂₆F₃N₅O₃ requires 509. Ret. time 1.91 min.

¹H NMR (d⁶DMSO) δ: 1.28-1.48 (3H, m), 1.70-1.83 (3H, m), 1.83-2.12 (4H, m), 2.49 (3H, s), 4.42 (2H, s), 7.38-7.47 (1H, m), 7.58 (1H, t), 7.72-7.87 (2H, m), 7.97 (1H, d), 8.04 (1H, s), 8.14 (1H, s), 8.50-8.60 (2H, m), 9.58 (1H, s), 10.75 (1H, s).

### EXAMPLE 22

### 2-{3-[4-(1-Methyl-1H-imidazol-2-yl)phenyl]-2-oxo-1,4-diazaspiro[4.4]non-3-en-1-yl}-N-[3-(trifluoromethyl)phenyl]acetamide hydrochloride

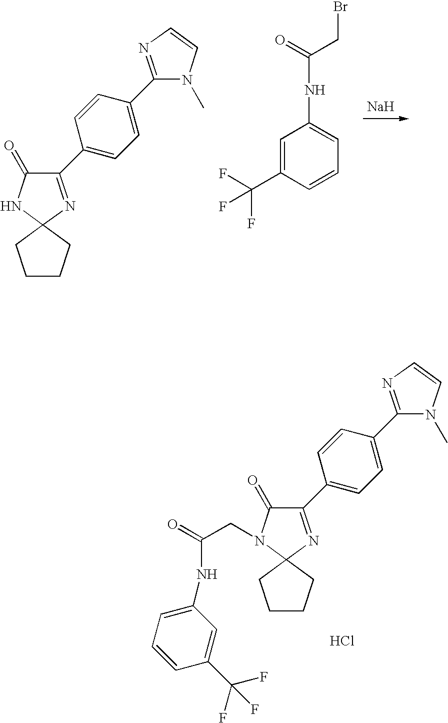

Sodium hydride in mineral oil (2.283 mg, 0.057 mmol) was added to a, stirring, ice-bath cooled solution of 3-[4-(1-methyl-1H-imidazol-2-yl)phenyl]-1,4-diazaspiro[4.4]non-3-en-2-one (D32) (14 mg) in DMF (2 ml) under argon. After stirring for 30 min. a solution of 2-bromo-N-[3-(trifluoromethyl)phenyl]acetamide (13.42 mg) in DMF (1 ml) was added over 1 h using a syringe pump maintaining an ice-bath temperature. The resulting reaction mixture was allowed to come to room temperature then stirred for a further 1 h. The reaction mixture was diluted with MeOH (5 ml) and passed through an SCX column (2 g). After eluting with MeOH (20 ml) the partially purified product was eluted with 2M NH₃-MeOH (5 ml). Evaporation afforded a cream solid which was purified by MDAP to afford the free-base of the title compound (70% purity).

¹H NMR) δ: 1.70-2.25 (8H, bm), 3.81 (3H, s), 4.27 (2H, s), 7.03 (1H, s), 7.18 (1H, s), 7.35 (1H, m), 7.40 (1H, m), 7.64 (1H, m), 7.80 (2H, m), 7.84 (1H, m), 8.54 (2H, m), 9.28 (1H, bs).

Mass Spectrum (Electrospray LC/MS): Found 496 (MH⁺). C₂₆H₂₄F₃N₅O₂ requires 495. Ret. time 1.70 min.

A solution of the free-base (14 mg, 0.028 mmol) in DCM (5 ml) was treated with an excess of 1M HCl in diethyl ether (0.94 ml, 0.94 mmol). The volatiles were removed in vacuo and the residue dried under high vac at 50° C. to afford the title compound as a pale yellow solid (15 mg).

Mass Spectrum (Electrospray LC/MS): Found 496 (MH⁺). C₂₅H₂₄ClN₅O₂ requires 495. Ret. time 1.63 min.

1-22. (canceled)

22. A compound of formula (I) or a salt thereof: 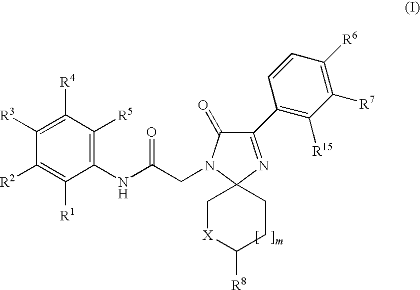
wherein:
X is —CH₂— or oxygen;
R¹, R², R³ and R⁴ are independently selected from the group consisting of hydrogen, C₁₋₄alkyl, C₁₋₄alkoxy, cyano, halo, haloC₁₋₄alkyl, haloC₁₋₄alkoxy, C₁₋₄alkylthio, C₃₋₆cycloalkyl, C₃₋₆cycloalkylC₁₋₄alkyl, C₃₋₆cycloalkylC₁₋₄alkoxy, C₁₋₄alkylsulfonyl, C₁₋₄alkoxyC₁₋₄alkyl and CONR^{a}R^{b} wherein R^{a} and R^{b} are independently selected from hydrogen and C₁₋₄alkyl, or R^{a} and R^{b}, together with the nitrogen atom to which they are attached, form a 4- to 7-membered ring;
or R² and R³ together form a group which is —O—CH₂—O— or —O—CH₂—CH₂—O—;
R⁵ is hydrogen, chloro, fluoro, C₁₋₄alkyl or CF₃;
one of R⁶ and R⁷ is selected from the group consisting of:
hydrogen, C₁₋₄alkyl, C₁₋₄alkoxy, haloC₁₋₄alkyl, halo C₁₋₄alkoxy, halo, cyano, C₁₋₄alkoxyC₁₋₄alkoxy and C₁₋₄alkoxyC₁₋₄alkyl;
and the other is selected from the group consisting of:
a 5 to 7 membered heteroaryl ring, optionally substituted by C₁₋₄alkyl, C₁₋₄alkoxy, haloC₁₋₄alkyl, haloC₁₋₄alkoxy, halo or cyano;
a 9 to 10 membered bicyclic heterocyclic ring, optionally substituted by C₁₋₄alkyl, C₁₋₄alkoxy, haloC₁₋₄alkyl, haloC₁₋₄alkoxy, halo or cyano; and
a 5 to 7 membered heterocyclic ring, optionally substituted by C₁₋₄alkyl, C₁₋₄alkoxy, haloC₁₋₄alkyl, haloC₁₋₄alkoxy, halo or cyano;
or R⁶ and R⁷ together form a 5 to 7 membered heterocyclic ring fused to the phenyl ring, or a 5 to 7 membered heteroaryl ring fused to the phenyl ring; wherein the heterocyclic ring or the heteroaryl ring is optionally substituted by C₁₋₄alkyl, C₁₋₄alkoxy, haloC₁₋₄alkyl, haloC₁₋₄alkoxy, halo or cyano;
R¹⁵ is hydrogen or fluorine;
R⁸ is hydrogen or methyl; and
m is selected from 0, 1 and 2.

23. A compound as claimed in claim 22 wherein R¹ is hydrogen.

24. A compound as claimed in claim 22 wherein R² is halo or haloC₁₋₂alkyl.

25. A compound as claimed in claim 22 wherein R³ is hydrogen.

26. A compound as claimed in claim 22 wherein R⁴ is hydrogen or halo.

27. A compound as claimed in claim 22 wherein R⁵ is hydrogen.

28. A compound as claimed in claim 22 wherein R¹, R³ and R⁵ are all hydrogen; R² is F or CF₃; and R⁴ is F or H.

29. A compound as claimed in claim 22 wherein:
R⁷ is hydrogen and R⁶ is a 5 or 6 membered heteroaryl ring, optionally substituted by C₁₋₄alkyl, C₁₋₄alkoxy, haloC₁₋₄alkyl, haloC₁₋₄alkoxy, halo or cyano; or
R⁶ is H and R⁷ is a 5 or 6 membered heteroaryl ring, optionally substituted by C₁₋₄alkyl, C₁₋₄alkoxy, haloC₁₋₄alkyl, haloC₁₋₄alkoxy, halo or cyano.

30. A compound as claimed in claim 22 wherein R¹⁵ is hydrogen.

31. A compound as claimed in claim 22 wherein R⁸ is hydrogen.

32. A compound as claimed in claim 22 wherein m is 1 or 0.

33. A compound as claimed in claim 1, which is selected from the group consisting of:
2-{3-[4-(4-Methyl-1H-imidazol-1-yl)phenyl]-2-oxo-1,4-diazaspiro[4.4]non-3-en-1-yl}-N-[3-(trifluoromethyl)phenyl]acetamide;
2-{3-[4-(5-Methyl-1H-imidazol-1-yl)phenyl]-2-oxo-1,4-diazaspiro[4.4]non-3-en-1-yl}-N-[3-(trifluoromethyl)phenyl]acetamide;
2-{3-[4-(4-methyl-1H-imidazol-1-yl)phenyl]-2-oxo-1,4-diazaspiro[4.5]dec-3-en-1-yl}-N-[3-(trifluoromethyl)phenyl]acetamide;
2-{3-[4-(5-methyl-1H-imidazol-1-yl)phenyl]-2-oxo-1,4-diazaspiro[4.5]dec-3-en-1-yl}-N-[3-(trifluoromethyl)phenyl]acetamide;
2-{3-[4-(1H-imidazol-1-yl)phenyl]-2-oxo-1,4-diazaspiro[4.5]dec-3-en-1-yl}-N-[3-(trifluoromethyl)phenyl]acetamide;
2-{3-[4-(2-methyl-1H-imidazol-1-yl)phenyl]-2-oxo-1,4-diazaspiro[4.5]dec-3-en-1-yl}-N-[3-(trifluoromethyl)phenyl]acetamide;
2-{2-Oxo-3-[4-(2-propyl-1H-imidazol-1-yl)phenyl]-1,4-diazaspiro[4.5]dec-3-en-1-yl}-N-[3-(trifluoromethyl)phenyl]acetamide;
N-(3,5-Difluorophenyl)-2-{3-[4-(4-methyl-1H-imidazol-1-yl)phenyl]-2-oxo-1,4-diazaspiro[4.4]non-3-en-1-yl}acetamide;
2-{3-[4-(4-Methyl-1H-imidazol-1-yl)phenyl]-2-oxo-7-oxa-1,4-diazaspiro[4.4]non-3-en-1-yl}-N-[3-(trifluoromethyl)phenyl]acetamide;
2-{3-[4-(4-Methyl-1H-imidazol-1-yl)phenyl]-2-oxo-7-oxa-1,4-diazaspiro[4.4]dec-3-en-1-yl}-N-[3-(trifluoromethyl)phenyl]acetamide;
2-{3-[4-(5-methyl-1H-imidazol-1-yl)phenyl]-2-oxo-7-oxa-1,4-diazaspiro[4.5]dec-3-en-1-yl}-N-[3-(trifluoromethyl)phenyl]acetamide ;
2-{2-Oxo-3-[4-(2-pyridinyl)phenyl]-7-oxa-1,4-diazaspiro[4.4]non-3-en-1-yl}-N-[3-(trifluoromethyl)phenyl]acetamide;
2-{2-Oxo-3-[4-(2-pyridinyl)phenyl]-1,4-diazaspiro[4.4]non-3-en-1-yl}-N-[3-(trifluoromethyl)phenyl]acetamide;
N-(3-Chlorophenyl)-2-{3-[4-(4-methyl-1H-imidazol-1-yl)phenyl]-2-oxo-1,4-diazaspiro[4.4]non-3-en-l-yl}acetamide;
2-{2-Oxo-3-[3-(2-pyridinyl)phenyl]-1,4-diazaspiro[4.5]dec-3-en-1-yl}-N-[3-(trifluoromethyl)phenyl]acetamide;
2-{2-Oxo-3-[4-(2-pyridinyl)phenyl]-7-oxa-1,4-diazaspiro[4.5]dec-3-en-1-yl}-N-[3-(trifluoromethyl)phenyl]acetamide;
N-(3,5-Difluorophenyl)-2-{2-oxo-3-[4-(2-pyridinyl)phenyl]-7-oxa-1,4-diazaspiro[4.5]dec-3-en-1-yl}acetamide;
2-{3-[4-(2-Methyl-1H-imidazol-1-yl)phenyl]-2-oxo-1,4-diazaspiro[4.4]non-3-en-1-yl}-N-[3-(trifluoromethyl)phenyl]acetamide;
2-{3-[4-(2,4-Dimethyl-1H-imidazol-1-yl)phenyl]-2-oxo-1,4-diazaspiro[4.4]non-3-en-1-yl}-N-[3-(trifluoromethyl)phenyl]acetamide ;
2-{3-[4-(4,5-Dimethyl-1H-imidazol-1-yl)phenyl]-2-oxo-1,4-diazaspiro[4.4]non-3-en-1-yl}-N-[3-(trifluoromethyl)phenyl]acetamide ;
2-{3-[3-(4-Methyl-1H-imidazol-1-yl)phenyl]-2-oxo-1,4-diazaspiro[4.5]dec-3-en-1-yl}-N-[3-(trifluoromethyl)phenyl]acetamide;
2-{3-[3-(5-Methyl-1H-imidazol-1-yl)phenyl]-2-oxo-1,4-diazaspiro[4.5]dec-3-en-1-yl}-N_[3-(trifluoromethyl)phenyl]acetamide;
2-{3-[4-(1-Methyl-1H-imidazol-2-yl)phenyl]-2-oxo-1,4-diazaspiro[4.4]non-3-1-yl}-N-[3-(trifluoromethyl)phenyl]acetamide;
or a salt thereof

34. A method of treating schizophrenia, dementia or attention deficit disorder comprising administering to a patient in need thereof an effective amount of a compound of formula (I) as defined in claim 22 or a pharmaceutically acceptable salt thereof

35. A pharmaceutical composition comprising a compound of formula (I) as defined in claim 1 or a pharmaceutically acceptable salt thereof and at least one pharmaceutically acceptable excipient.
